SEQUENCE LISTING (44 sequences)

SEQ ID NO: 1 - protein, 16 aa, Artificial Sequence
  A16Y T-helper epitope
AQYIKANSKFIGITEL

SEQ ID NO: 2 - protein, 23 aa, Artificial Sequence
  RSV SHeA
NKLCEYNVFHNKTFELPRARVNT

SEQ ID NO: 3 - protein, 24 aa, Artificial Sequence
  RSV SHeB
NKLSEHKTFCNKTLEQGQMYQINT

SEQ ID NO: 4 - protein, 9 aa, Artificial Sequence
  Survivin HLA-A1 (modified)
FTELTLGEF

SEQ ID NO: 5 - protein, 9 aa, Artificial Sequence
  Survivin HLA-A2 (modified)
LMLGEFLKL

SEQ ID NO: 6 - protein, 10 aa, Artificial Sequence
  Survivin HLA-A3 (modified)
RISTFKNWPK

SEQ ID NO: 7 - protein, 9 aa, Artificial Sequence
  Survivin HLA-A24
STFKNWPFL

SEQ ID NO: 8 - protein, 9 aa, Artificial Sequence
  Survivin HLA-B7
LPPAWQPFL

SEQ ID NO: 9 - protein, 9 aa, Artificial Sequence
  HPV 16E7
RAHYNIVTF

SEQ ID NO: 10 - protein, 9 aa, Artificial Sequence
  HPV Y9T
YMLNLGPET

SEQ ID NO: 11 - protein, 10 aa, Artificial Sequence
  HIV RGP10
RGPGRAFVTI

SEQ ID NO: 12 - protein, 9 aa, Artificial Sequence
  HIV AMQ9
AMQMLKETI

SEQ ID NO: 13 - protein, 64 aa, Artificial Sequence
  RSV Subtype A
MENTSITIEFSSKFWPYFTLIHMITTIISLLIIISIMIAILNKLCEYNVFHNKTFELPRARVNT

SEQ ID NO: 14 - protein, 65 aa, Artificial Sequence
  RSV Subtype B
MGNTSITIEFTSKFWPYFTLIHMILTLISLLIIITIMIAILNKLSEHKTFCNKTLEQGQMYQINT

SEQ ID NO: 15 - protein, 23 aa, Artificial Sequence
  RSV SHeA C45S
NKLSEYNVFHNKTFELPRARVNT

SEQ ID NO: 16 - protein, 40 aa, Artificial Sequence
  RSV bSHeA
NKLCDLNDHHTNSLDIRTRLRNDTQLITRAHEGSINQSSN

SEQ ID NO: 17 - protein, 40 aa, Artificial Sequence
  RSV bSHeA C45S
NKLSDLNDHHTNSLDIRTRLRNDTQLITRAHEGSINQSSN

SEQ ID NO: 18 - protein, 24 aa, Artificial Sequence
  RSV SHeB C51S
NKLSEHKTFSNKTLEQGQMYQINT

SEQ ID NO: 19 - protein, 24 aa, Artificial Sequence
  RSV SHeB C45S
NKLCEHKTFSNKTLEQGQMYQINT

SEQ ID NO: 20 - protein, 29 aa, Artificial Sequence
  RSV L-SHeB C51S
CGGGSNKLSEHKTFSNKTLEQGQMYQINT

SEQ ID NO: 21 - DNA, 429 bp, Homo sapiens
atgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattga

SEQ ID NO: 22 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 23 - protein, 9 aa, Artificial Sequence
  Survivin HLA-A1
FEELTLGEF

SEQ ID NO: 24 - protein, 9 aa, Artificial Sequence
  Survivin HLA-A2
LTLGEFLKL

SEQ ID NO: 25 - protein, 10 aa, Artificial Sequence
  Survivin HLA-A3
RISTFKNWPF

SEQ ID NO: 26 - protein, 27 aa, Artificial Sequence
  Mut25
STANYNTSHLNNDVWQIFENPVDWKEK

SEQ ID NO: 27 - protein, 27 aa, Artificial Sequence
  Mut30
PSKPSFQEFVDWENVSPELNSTDQPFL

SEQ ID NO: 28 - protein, 27 aa, Artificial Sequence
  Mut44
EFKHIKAFDRTFANNPGPMVVFATPGM

SEQ ID NO: 29 - protein, 13 aa, Artificial Sequence
  PADRE T-helper epitope
  misc_feature (3)..(3): Xaa may be cyclohexylalanyl
AKXVAAWTLKAAA

SEQ ID NO: 30 - protein, 21 aa, Artificial Sequence
  F21E T-helper epitope
FNNFTVSFWLRVPKVSASHLE

SEQ ID NO: 31 - DNA, 20 bp, Artificial Sequence
  CpG oligonucleotide
tccatgacgttcctgacgtt

SEQ ID NO: 32 - DNA, 26 bp, Artificial Sequence
  PolyIC oligonucleotide (dIdC)
  modified_base (1)..(1): Inosine
  misc_feature (1)..(1): n is a, c, g, or t
  modified_base (3)..(3): Inosine
  misc_feature (3)..(3): n is a, c, g, or t
  modified_base (5)..(5): Inosine
  misc_feature (5)..(5): n is a, c, g, or t
  modified_base (7)..(7): Inosine
  misc_feature (7)..(7): n is a, c, g, or t
  modified_base (9)..(9): Inosine
  misc_feature (9)..(9): n is a, c, g, or t
  modified_base (11)..(11): Inosine
  misc_feature (11)..(11): n is a, c, g, or t
  modified_base (13)..(13): Inosine
  misc_feature (13)..(13): n is a, c, g, or t
  modified_base (15)..(15): Inosine
  misc_feature (15)..(15): n is a, c, g, or t
  modified_base (17)..(17): Inosine
  misc_feature (17)..(17): n is a, c, g, or t
  modified_base (19)..(19): Inosine
  misc_feature (19)..(19): n is a, c, g, or t
  modified_base (21)..(21): Inosine
  misc_feature (21)..(21): n is a, c, g, or t
  modified_base (23)..(23): Inosine
  misc_feature (23)..(23): n is a, c, g, or t
  misc_feature (25)..(25): n is a, c, g, or t
ncncncncncncncncncncncncnc

SEQ ID NO: 33 - protein, 6 aa, Artificial Sequence
  Palmitic Acid Adjuvant
  misc_feature (1)..(1): Linked to PAM2 or PAM3
CSKKKK

SEQ ID NO: 34 - protein, 27 aa, Artificial Sequence
  Mut17
VVDRNPQFLDPVLAYLMKGLCEKPLAS

SEQ ID NO: 35 - protein, 27 aa, Artificial Sequence
  Mut20
FRRKAFLHWYTGEAMDEMEFTEAESNM

SEQ ID NO: 36 - protein, 27 aa, Artificial Sequence
  Mut22
PKPDFSQLQRNILPSNPRVTRFHINWD

SEQ ID NO: 37 - protein, 27 aa, Artificial Sequence
  Mut24
TAVITPPTTTTKKARVSTPKPATPSTD

SEQ ID NO: 38 - protein, 27 aa, Artificial Sequence
  Mut28
NIEGIDKLTQLKKPFLVNNKINKIENI

SEQ ID NO: 39 - protein, 27 aa, Artificial Sequence
  Mut29A
IPSGTTILNCFHDVLSGKLSGGSPGVP

SEQ ID NO: 40 - protein, 27 aa, Artificial Sequence
  Mut29B
SFACLRQPSQGPTVGVKGGAAGGGYAQ

SEQ ID NO: 41 - protein, 27 aa, Artificial Sequence
  Mut36
CGTAFFINFIAIYHHASRAIPFGTMVA

SEQ ID NO: 42 - protein, 27 aa, Artificial Sequence
  Mut45
ECRITSNFVIPSEYWVEEKEEKQKLIQ

SEQ ID NO: 43 - protein, 27 aa, Artificial Sequence
  Mut48
SHCHWNDLAVIPAGVVHNWDFEPRKVS

SEQ ID NO: 44 - protein, 27 aa, Artificial Sequence
  Mut50
GFSQPLRRLVLHVVSAAQAERLARAEE

(19) United States
(12) Patent Application Publication
(10) Pub. No.: US 2020/0230057 A1
(43) Pub. Date: Jul. 23, 2020

(54) Pharmaceutical Compositions, Methods for Preparation using Lipid Vesicle Particles of Defined Size, and Uses Thereof

(71) Applicant: IMMUNOVACCINE TECHNOLOGIES INC., Dartmouth (CA)

(72) Inventors: Leeladhar SAMMATUR, Halifax (CA); Rajkannan RAJAGOPALAN, Dartmouth (CA); Arthvan SHARMA, Halifax (CA); Valarmathy KALIAPERUMAL, Halifax (CA); Genevieve WEIR, Lawrencetown (CA); Marianne STANFORD, Upper Tantallon (CA); Andrea PENWELL, Fall River (CA)

(73) Assignee: IMMUNOVACCINE TECHNOLOGIES INC., Dartmouth, NS (CA)

(21) Appl. No.: 16/629,780

(22) PCT Filed: Nov. 9, 2017
(86) PCT No.: PCT/CA2017/051336

§ 371 Date: Jan. 9, 2020

Related U.S. Application Data
(60) Provisional application No. 62/530,498, filed on Jul. 10, 2017.

(51) Int. Cl. A61K 9/127; A61K 39/12; A61K 39/00; A61K 39/07; A61K 9/19

(52) U.S. Cl. CPC A61K 9/127; A61K 39/12; A61K 2039/55555; A61K 39/07; A61K 9/19; A61K 39/00115

(57) ABSTRACT

The present disclosure relates to methods for preparing a dried preparation comprising lipids and a therapeutic agent by using lipid vesicle particles having a mean particle size of ≤120 nm and a polydispersity index (PDI) of ≤0.1. The present application also provides stable, water-free pharmaceutical compositions comprising one or more lipid-based structures having a single layer lipid assembly, at least one therapeutic agent, and a hydrophobic carrier, as well as methods of treatment, uses and kits relating thereto, such as for example for inducing an antibody and/or CTL immune response.

### CROSS REFERENCE TO RELATED APPLICATIONS

This application claims the benefit of and priority to U.S. Provisional Patent Application No. 62/530,498 filed on Jul. 10, 2017, which is hereby incorporated by reference in its entirety.

### FIELD

The present application relates to methods for preparing a dried preparation comprising lipids and a therapeutic agent, methods for preparing pharmaceutical compositions, and stable, water-free pharmaceutical compositions comprising one or more lipid-based structures having a single layer lipid assembly, at least one therapeutic agent, and a hydrophobic carrier.

### BACKGROUND

In the pharmaceutical field, the effective delivery of therapeutic agents often poses difficulties and challenges, particularly in respect of the complexities of emerging delivery platforms designed to enhance the efficacy of therapeutic agents. For these specialized delivery platforms that employ unique components, new hurdles arise that do not exist for conventional pharmaceutical compositions. This is certainly the case for delivery platforms using water-free, hydrophobic carriers.

Various characteristics of therapeutic agents make their incorporation into such delivery platforms a time-consuming and labour intensive feat, particularly when multiple different therapeutic agents must be formulated together in a single composition. For instance, because of the high degree of hydrophilicity or hydrophobicity of many therapeutic agents, manufacturing processes involving sequential stages of preparation in both aqueous and hydrophobic solutions create unique obstacles for preparing pharmaceutical grade formulations. Moreover, encapsulation of therapeutic agents in liposomal delivery vehicles means size extrusion steps are often required in order to effectively perform sterile filtration procedures to obtain pharmaceutical grade compositions. However, these extrusion steps can cause therapeutic agents to come out of solution, resulting in a lack of reproducibility and/or an unacceptable composition for pharmaceutical purposes.

These challenges are compounded when it is desirable to formulate pharmaceutical compositions with multiple different therapeutic agents, such as in the context of personalized cancer vaccines.

Personalized cancer vaccines can be designed based on patient-specific neoantigens, which are tumor specific antigens that arise via mutations (non-synonymous somatic mutations) altering amino acid coding sequences. Recently, with the aid of sequencing technologies, it has become possible to identify the mutations present within the protein-encoding part of the genome (the exome) of an individual tumor and thereby predict potential neoantigens. However, neoantigen-based personalized cancer vaccines typically have to be formulated within weeks to facilitate an effective immune response upon immunization. Timing is critical and poses significant hurdles. The time involved in first selecting potential neoantigens and then, within this population, selecting peptides having suitable characteristics to formulate stable compositions, has made progress in this field difficult at best.

With the aim of providing suitable treatments, neoantigen vaccines currently being explored in clinical trials deliver 10-30 individual peptides in multiple formulations. While this approach reduces the time involved in selecting the most appropriate neoantigen, there remain significant issues with respect to formulation stability and/or immunogenicity, cost and patient compliance. Ideally, the antigens could be delivered in a single, stable formulation without time-consuming efforts in selecting antigens that have a particular set of characteristics relating to isoelectric point, stability, solubility (e.g. co-solubility) and/or immunogenicity. But in reality, identifying a perfect formulation method for each peptide antigen based on its unique nature is a strenuous and time consuming process.

As such, there remains a need for a robust and time-sensitive manufacturing method to formulate therapeutic agents in a stable and immunologically effective pharmaceutical composition. There also remains a need for stable and effective pharmaceutical compositions comprising multiple therapeutic agents.

### SUMMARY

In an embodiment, the present disclosure relates to a method for preparing a dried preparation comprising lipids and a therapeutic agent, said method comprising the steps of: (a) providing lipid vesicle particles having a mean particle size of ≤120 nm and a polydispersity index (PDI) of ≤0.1; (b) mixing the lipid vesicle particles with at least one solubilized therapeutic agent to form a mixture; and (c) drying the mixture formed in step (b) to form a dried preparation comprising lipids and a therapeutic agent.

In an embodiment, the present disclosure relates to a method for preparing a pharmaceutical composition comprising solubilizing the dried preparation obtained by the method as described herein in a hydrophobic carrier.

In an embodiment, the present disclosure relates to a pharmaceutical composition prepared by the method as disclosed herein.

In an embodiment, the present disclosure relates to a stable, water-free pharmaceutical composition comprising one or more lipid-based structures having a single layer lipid assembly, at least one therapeutic agent, and a hydrophobic carrier.

In an embodiment, the present disclosure relates to a stable, water-free pharmaceutical composition comprising one or more lipid-based structures having a single layer assembly, five or more different peptide neoantigens, and a hydrophobic carrier, wherein the peptide neoantigens are not pre-selected based on any characteristic relating to isoelectric point, solubility, stability and/or immunogenicity.

In an embodiment, the present disclosure relates to a method of inducing an antibody and/or CTL immune response in a subject comprising administering to the subject the pharmaceutical composition as described herein.

In an embodiment, the present disclosure relates to the use of the pharmaceutical composition as described herein for inducing an antibody and/or CTL immune response in a subject.

In an embodiment, the present disclosure relates to a kit for preparing a pharmaceutical composition for inducing an antibody and/or CTL immune response, the kit comprising: a container comprising a dried preparation prepared by the method as described herein; and a container comprising a hydrophobic carrier.

Other aspects and features of the present invention will become apparent to those of ordinary skill in the art upon review of the following description in conjunction with the accompanying figures.

### BRIEF DESCRIPTION OF THE FIGURES

The accompanying figures, which constitute a part of this specification, illustrate embodiments of the invention by way of example only:

FIG. 1 is a photograph of a dried lipid/therapeutic agent preparation prepared using sodium phosphate buffer, 100 mM, pH 7.0 (left panel) and a pharmaceutical composition obtained therefrom upon solubilization (right panel).

FIG. 2 is a photograph of a dried lipid/therapeutic agent preparation prepared using sodium phosphate buffer, 50 mM, pH 7.0 (left panel) and a pharmaceutical composition obtained therefrom upon solubilization (right panel).

FIG. 3 is a photograph of a another dried lipid/therapeutic preparation prepared using sodium phosphate buffer, 50 mM, pH 7.0 (left panel) and a pharmaceutical composition obtained therefrom upon solubilization (right panel).

FIG. 4 depicts photographs of dried lipid/therapeutic agent preparations and pharmaceutical compositions obtained therefrom upon solubilization. Panel (A): sized lipid vesicle particles and rPA antigen, Panel (B): sized lipid vesicle particles and HIV antigen, Panel (C): sized lipid vesicle particles and survivin antigen, Panel (D): sized lipid vesicle particles and RSV antigen, Panel (E): non-sized lipid vesicle particles and rPA antigen, Panel (F): non-sized lipid vesicle particles and HIV antigen, Panel (G): non-sized lipid vesicle particles and survivin antigen, Panel (H): non-sized lipid vesicle particles and RSV antigen.

FIG. 5 depicts photographs of dried lipid/therapeutic agent preparations and pharmaceutical compositions obtained therefrom upon solubilization. Panel (A): DOPC/cholesterol sized lipid vesicle particles and survivin antigen, Panel (B): DOPC/cholesterol sized lipid vesicle particles and RSV antigen, Panel (C): DOPC alone sized lipid vesicle particles and survivin antigen, Panel (D): DOPC alone sized lipid vesicle particles and RSV antigen.

FIG. 6 is a photograph of aseptically filled vials of the sized lipid vesicle particle/antigen mixture loaded in a tray for lyophilization prior to being sealed in a sterile bag.

FIG. 7 is a photograph of the aseptically filled vials of FIG. 6 loaded into two sterile bags and sealed, in preparation for lyophilization.

FIG. 8 is a photograph of the double sterile bags vials of FIG. 7 inside a Virtis Benchtop Lyophilizer for lyophilization.

FIG. 9 is a photograph of the double sterile bags vials of FIG. 7 tray stoppered inside a Virtis Benchtop Lyophilizer after lyophilization.

FIG. 10 is a photograph of five vials after lyophilization by the sterile bag technique, and one vial of pharmaceutical composition obtained therefrom upon solubilization. The pharmaceutical composition is a clear solution.

FIG. 11 is a photograph of a dried lipid/therapeutic agent preparation prepared using sodium acetate buffer, 50 mM, pH 6.0 (left and middle panels) and a pharmaceutical composition obtained therefrom upon solubilization (right panel).

FIG. 12 is an HPLC chromatogram of a standard sample showing 14 neoantigens and A16L T-helper epitope.

FIG. 13 is an HPLC chromatogram showing 14 neoantigens and A16L T-helper epitope before lyophilization.

FIG. 14 is an HPLC chromatogram showing 14 neoantigens and A16L T-helper epitope immediately after lyophilization (T=0).

FIG. 15 is an HPLC chromatogram showing 14 neoantigens and A16L T-helper epitope 1 month after lyophilization (T=1M).

FIG. 16 is an HPLC chromatogram showing 14 neoantigens and A16L T-helper epitope 3 months after lyophilization (T=3M).

FIG. 17 illustrates the IFN-gamma ELISPOT responses of C57BL/6NCrl mice vaccinated with 14 different neoantigens prepared in either an oil-based composition of the invention or an aqueous formulation. Immune responses were measured eight days after vaccination by stimulating splenocytes with syngeneic dendritic cells unloaded or loaded with an irrelevant peptide or each individual neoantigen (Mut 17, 20, 22, 24, 25, 28, 29A, 29B, 30, 36, 44, 45, 48, 50) in an IFN-gamma ELISPOT plate. Results are shown as average response±SEM. Statistical analysis was performed by 2-way ANOVA with Bonferroni post test comparing group responses to each peptide. **p<0.01, ***p<0.001.

FIG. 18 depicts the small angle x-ray scattering (SAXS) pattern for a sample of Montanide ISA 51 VG.

FIG. 19 depicts the SAXS pattern for a sample of Batch #4c (survivin). Also shown is the pair distance distribution function for the Batch #4c sample at 27.3 cm detector distance.

FIG. 20 depicts the SAXS pattern for a sample of Batch #4a (rPA). Also shown is the pair distance distribution function for the Batch #4a sample at 27.3 cm detector distance.

FIG. 21 depicts photographs of pharmaceutical compositions prepared in an MS80 oil carrier using sized lipid vesicle particles of DOPC/Cholesterol or S100 Lecithin. Panel (A): DOPC/cholesterol sized lipid vesicle particles and rPA antigen; Panel (B): DOPC/cholesterol sized lipid vesicle particles and RSV antigen; Panel (C): S100 Lecithin sized lipid vesicle particles and RSV antigen; Panel (D): S100 Lecithin sized lipid vesicle particles and survivin antigens; Panel (E): S100 Lecithin sized lipid vesicle particles and malaria antigen; Panel (F): S100 Lecithin/cholesterol (10:1 w/w) sized lipid vesicle particles and HIV antigen. All resulted in a clear solution. For compositions using S100 Lecithin, the solutions were slightly yellow in color because the S100 material is yellow.

FIG. 22 is a photograph of a dried lipid/therapeutic agent preparation prepared using sized lipid vesicle particles of DOPC/Cholesterol and a small molecule therapeutic agent, cyclophosphamide (left panel); and a pharmaceutical composition obtained therefrom upon solubilization (right panel). The dried preparation had a good appearance in that it was dry, white and non-collapsed. The composition obtained therefrom was a clear to slightly hazy solution.

### DETAILED DESCRIPTION

The present invention relates to advantageous methods for preparing a dried preparation comprising lipids and a therapeutic agent, as well as pharmaceutical compositions prepared therefrom. The disclosed methods avoid time-consuming processing steps and allow for the rapid production of a pharmaceutical composition containing multiple therapeutic agents, which is expected to be particularly useful, for example, in the context of personalized cancer vaccines involving peptide antigens (e.g. patient-specific neoantigens).

Method for Preparing a Dried Lipid/Therapeutic Agent Preparation

In an embodiment, the present invention relates to a method for preparing a dried preparation comprising lipids and a therapeutic agent, said method comprising the steps of: (a) providing lipid vesicle particles having a mean particle size of ≤120 nm and a polydispersity index (PDI) of ≤0.1; (b) mixing the lipid vesicle particles with at least one solubilized therapeutic agent to form a mixture; and (c) drying the mixture formed in step (b) to form a dried preparation comprising lipids and a therapeutic agent. The method steps are to be performed in this specific order, but the method may comprise additional steps, such as for example those described herein, without limitation.

As used herein, the term “lipid vesicle particle” may be used interchangeably with “lipid vesicle”. A lipid vesicle particle refers to a complex or structure having an internal environment separated from the external environment by a continuous layer of enveloping lipids. In the context of the present disclosure, the expression “layer of enveloping lipids” can mean a single layer lipid membrane (e.g. as found on a micelle or reverse micelle), a bilayer lipid membrane (e.g. as found on a liposome) or any multilayer membrane formed from single and/or bilayer lipid membranes. The layer of enveloping lipids is typically a single layer, bilayer or multilayer throughout its circumference, but it is contemplated that other conformations may be possible such that the layer has different configurations over its circumference. The lipid vesicle particle may contain, within its internal environment, other vesicle structures (i.e. it may be multivesicular).

The term “lipid vesicle particle” encompasses many different types of structures, including without limitation micelles, reverse micelles, unilamellar liposomes, multilamellar liposomes and multivesicular liposomes, so long as the particle size limitations described herein are met (i.e. a mean particle size of ≤120 nm and a PDI of ≤0.1).

Depending on the definition ascribed to lipid nanoparticles, the lipid vesicle particles of the present disclosure may be synonymous with lipid nanoparticles. However, there are contrasting views in the art on the meaning of the term “lipid nanoparticle”. One view is that a lipid nanoparticle refers to any nano-sized particle (i.e. having a diameter of between 1 nanometer and 1000 nanometers) formed by a lipid membrane. Another view is that the size threshold for a nanoparticle material is limited to between 1 nanometer and 100 nanometers. This latter definition excludes lipid vesicle sizes that are encompassed by the present disclosure (e.g. lipid vesicle particles >100 nm in size), and to this extent is inconsistent with the term “lipid vesicle particles” as used in the present disclosure.

The lipid vesicle particles may take on various different shapes, and the shape may change at any given time (e.g. upon drying). Typically, lipid vesicle particles are spherical or substantially spherical structures. By “substantially spherical” it is meant that the lipid vesicles are close to spherical, but may not be a perfect sphere. Other shapes of the lipid vesicle particles include, without limitation, oval, oblong, square, rectangular, triangular, cuboid, crescent, diamond, cylinder or hemisphere shapes. Any regular or irregular shape may be formed. Further, a single lipid vesicle particle may comprise different shapes if it is multivesicular. For example, the outer vesicle shape may be oblong or rectangular while an inner vesicle may be spherical.

The lipid vesicle particles are formed from single layer lipid membranes, bilayer lipid membranes and/or multilayer lipid membranes. The lipid membranes are predominantly comprised of and formed by lipids, but may also comprise additional components. For example, and without limitation, the lipid membrane may include stabilizing molecules to aid in maintaining the size and/or shape of the lipid vesicle particle. Any stabilizing molecule known in the art may be used so long as it does not negatively affect the ability of the lipid vesicle particles to be used in the disclosed methods.

The term “lipid” has its common meaning in the art in that it is any organic substance or compound that is soluble in nonpolar solvents, but generally insoluble in polar solvents (e.g. water). Lipids are a diverse group of compounds including, without limitation, fats, waxes, sterols, fat-soluble vitamins, monoglycerides, diglycerides, triglycerides and phospholipids. For the lipid vesicle particles herein, any lipid may be used so long as it is a membrane-forming lipid. By “membrane-forming lipid” it is meant that the lipid, alone or together with other lipids and/or stabilizing molecules, is capable of forming the lipid membrane of the lipid vesicle particle. The lipid vesicle particles may comprise a single type of lipid or two or more different types of lipids.

In an embodiment, the lipid or lipids of the lipid vesicle particle are amphiphilic lipids, meaning that they possess both hydrophilic and hydrophobic (lipophilic) properties.

Although any lipid as defined above may be used, particularly suitable lipids may include those with at least one fatty acid chain containing at least 4 carbons, and typically about 4 to 28 carbons. The fatty acid chain may contain any number of saturated and/or unsaturated bonds. The lipid may be a natural lipid or a synthetic lipid. Non-limiting examples of lipids may include phospholipids, sphingolipids, sphingomyelin, cerobrocides, gangliosides, ether lipids, sterols, cardiolipin, cationic lipids and lipids modified with poly (ethylene glycol) and other polymers. Synthetic lipids may include, without limitation, the following fatty acid constituents: lauroyl, myristoyl, palmitoyl, stearoyl, arachidoyl, oleoyl, linoleoyl, erucoyl, or combinations of these fatty acids.

In an embodiment, the lipid is a phospholipid or a mixture of phospholipids. Broadly defined, a “phospholipid” is a member of a group of lipid compounds that yield on hydrolysis phosphoric acid, an alcohol, fatty acid, and nitrogenous base.

Phospholipids that may be used include for example, and without limitation, those with at least one head group selected from the group consisting of phosphoglycerol, phosphoethanolamine, phosphoserine, phosphocholine (e.g. DOPC; 1,2-Dioleoyl-sn-glycero-3-phosphocholine) and phosphoinositol. In an embodiment, the phospholipid may be phosphatidylcholine or a mixture of lipids comprising phosphatidylcholine. In an embodiment, the lipid may be DOPC (Lipoid GmbH, Germany) or Lipoid S100 lecithin. In some embodiments, a mixture of DOPC and unesterified cholesterol may be used. In other embodiments, a mixture of Lipoid S100 lecithin and unesterified cholesterol may be used.

In an embodiment, the lipid vesicle particles comprise a synthetic lipid. In an embodiment, the lipid vesicle particles comprise synthetic DOPC. In another embodiment, the lipid vesicle particles comprise synthetic DOPC and cholesterol.

When cholesterol is used, the cholesterol may be used in any amount sufficient to stabilize the lipids in the lipid membrane. In an embodiment, the cholesterol may be used in an amount equivalent to about 10% of the weight of phospholipid (e.g. in a DOPC:cholesterol ratio of 10:1 w/w). The cholesterol may stabilize the formation of phospholipid vesicle particles. If a compound other than cholesterol is used, one skilled in the art can readily determine the amount needed.

In an embodiment, the compositions disclosed herein comprise about 120 mg/ml of DOPC and about 12 mg/ml of cholesterol.

Another common phospholipid is sphingomyelin. Sphingomyelin contains sphingosine, an amino alcohol with a long unsaturated hydrocarbon chain. A fatty acyl side chain is linked to the amino group of sphingosine by an amide bond, to form ceramide. The hydroxyl group of sphingosine is esterified to phosphocholine. Like phosphoglycerides, sphingomyelin is amphipathic.

Lecithin, which also may be used, is a natural mixture of phospholipids typically derived from chicken eggs, sheep's wool, soybean and other vegetable sources.

All of these and other phospholipids may be used in the practice of the invention. Phospholipids can be purchased, for example, from Avanti lipids (Alabastar, Ala., USA), Lipoid LLC (Newark, N.J., USA) and Lipoid GmbH (Germany), among various other suppliers.

The lipid vesicle particles are closed vesicular structures. They are typically spherical in shape, but other shapes and conformations may be formed and are not excluded. Exemplary embodiments of lipid vesicle particles include, without limitation, single layer vesicular structures (e.g. micelles) and bilayer vesicular structures (e.g. unilamellar or multilamellar vesicles), or various combinations thereof.

By “single layer” it is meant that the lipids do not form a bilayer, but rather remain in a layer with the hydrophobic part oriented on one side and the hydrophilic part oriented on the opposite side. By “bilayer” it is meant that the lipids form a two-layered sheet, typically with the hydrophobic part of each layer internally oriented toward the center of the bilayer with the hydrophilic part externally oriented. However, the opposite configuration is also possible. The term “multilayer” is meant to encompass any combination of single and bilayer structures. The form adopted may depend upon the specific lipid that is used. Also, the forms used in the methods herein will depend on the size constraints of the disclosed method, i.e. a mean particle size of ≤120 nm and a PDI of ≤0.1.

In an embodiment, the lipid vesicle particle is a bilayer vesicular structure, such as for example, a liposome. Liposomes are completely closed lipid bilayer membranes. Liposomes may be unilamellar vesicles (possessing a single bilayer membrane), multilamellar vesicles (characterized by multimembrane bilayers whereby each bilayer may or may not be separated from the next by an aqueous layer) or multivesicular vesicles (possessing one or more vesicles within a vesicle). A general discussion of liposomes can be found in Gregoriadis 1990; and Frezard 1999.

Thus, in an embodiment, the lipid vesicle particles are liposomes. In an embodiment, the liposomes are unilamellar, multilamellar, multivesicular or a mixture thereof. In an embodiment, the mean particle size of the liposomes is ≥80 nm. Thus, in an embodiment, the mean particle size of the liposomes used in the methods disclosed herein is in the range of 80 nm to 120 nm, with a PDI of ≤0.1.

The methods disclosed herein require the use of lipid vesicle particles having a mean particle size of ≤120 nm and a polydispersity index (PDI) of ≤0.1.

As used herein, “mean” refers to the arithmetic mean of the particle size of the lipid vesicle particles in a given population. It is a synonym for average. As such, “mean particle size” is intended to refer to the sum of the diameters of each lipid vesicle particle of a population, divided by the total number of lipid vesicle particles in the population (e.g. in a population with 4 lipid vesicle particles with particle sizes of 95 nm, 98 nm, 102 nm and 99 nm, the mean particle size is (95+98+102+99)/4=98.5 nm). However, as the skilled person will appreciate, lipid vesicle particles may not be perfectly spherical, and therefore the “particle size” of a given vesicle particle may not be an exact measure of its diameter. Rather, the particle size may be defined by other means known in the art, including for example: the diameter of the sphere of equal area or the largest perpendicular distance between parallel tangents touching opposite sides of the particle (Feret's statistical diameter).

There are several techniques, instruments and services that are available to measure the mean particle size of lipid vesicle particles, such as electron microscopy (transmission, TEM, or scanning, SEM), atomic force microscopy (AFM), Fourier-transform infrared spectroscopy (FTIR), X-ray photoelectron spectroscopy (XPS), powder X-ray diffraction (XRD), matrix-assisted laser desorption/ionization time-of-flight mass spectrometry (MALDI-TOF-MS), nuclear magnetic resonance (NMR) and dynamic light scattering (DLS). DLS is a well-established technique for measuring the particle size in the submicron size range, with available technology to measure particle sizes of less than 1 nm (LS Instruments, CH; Malvern Instruments, UK).

In an embodiment, the mean particle size of ≤120 is measured by any instrument and/or machine suitable for measuring the mean particle size of lipid vesicle particles, such as by the methods above.

In an embodiment of the methods disclosed herein, the mean particle size is determined by DLS (Malvern Instruments, UK).

In an embodiment, the mean particle size of ≤120 is measured by DLS using a Malvern Zetasizer series instrument, such as for example the Zetasizer Nano S, Zetasizer APS, Zetasizer μV or Zetasizer AT machines (Malvern Instruments, UK). In an embodiment, the mean particle size of ≤120 is measured by DLS using a Malvern Zetasizer Nano S machine. Exemplary conditions and system settings may include:

Dispersant Name: 0.006% NaCl

Dispersant RI: 1.330

Viscosity (cP): 0.8872

Temperature (° C.): 25.0

Duration Used (s): 60

Count Rate (kcps): 200-400

Measurement Position (mm): 4.65

Cell Description: Disposable Sizing Cuvette

Attenuator: 7

In the methods herein, the lipid vesicle particles have a mean particle size of less than or equal to 120 nanometers (i.e. ≤120 nm) and a polydispersity index (PDI) of ≤0.1. In an embodiment, the lipid vesicle particles have a mean particle size of ≤115 nm, more particularly still ≤110 nm and more particularly still ≤100 nm. In an embodiment, the mean particle size of the lipid vesicle particles is between 50 nm and 120 nm. In an embodiment, the mean particle size of the lipid vesicle particles is between 80 nm and 120 nm. In an embodiment, the mean particle size of the lipid vesicle particles is between about 80 nm and about 115 nm, about 85 nm and about 115 nm, about 90 nm and about 115 nm, about 95 nm and about 115 nm, about 100 nm and about 115 nm or about 105 nm and about 115 nm.

In an embodiment, the mean particle size of the lipid vesicle particles is about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm or about 119 nm. In an embodiment, the mean particle size is 120 nm.

As used throughout herein, the term “about” means reasonably close. For example, “about” can mean within an acceptable standard deviation and/or an acceptable error range for the particular value as determined by one of ordinary skill in the art, which will depend on how the particular value is measured. Further, when whole numbers are represented, about can refer to decimal values on either side of the whole number. When used in the context of a range, the term “about” encompasses all of the exemplary values between the one particular value at one end of the range and the other particular value at the other end of the range, as well as reasonably close values beyond each end.

With respect to the mean particle size, the term “about” is used to represent a deviation of ±2.0 nm, so long as it would not cause the mean particle size to exceed 120 nm. Also, the term “about” is meant to encompass any decimal number of the indicated mean particle size.

In an embodiment, the mean particle size of the lipid vesicle particles is between about 105 nm and about 115 nm, such as for example when the lipid vesicle particles are formed from DOPC/cholesterol (10:1 w:w).

In an embodiment, the mean particle size of the lipid vesicle particles is between about 90 nm and about 100 nm, such as for example when the lipid vesicle particles are formed from DOPC alone.

As used herein, polydispersity index (PDI) is a measure of the size distribution of the lipid vesicle particles. It is known in the art that the term “polydispersity” may be used interchangeably with “dispersity”. The PDI can be calculated by determining the mean particle size of the lipid vesicle particles and the standard deviation from that size. There are techniques and instruments available for measuring the PDI of lipid vesicle particles. For example, DLS is a well-established technique for measuring the particle size and size distribution of particles in the submicron size range, with available technology to measure particle sizes of less than 1 nm (LS Instruments, CH; Malvern Instruments, UK).

For a perfectly uniform sample, the PDI would be 0.0. The methods disclosed herein require a PDI that is ≤0.1, which falls within the class of being “monodisperse”.

In an embodiment, the PDI of ≤0.1 is measured by any instrument and/or machine suitable for measuring the PDI of lipid vesicle particles.

In an embodiment of the PDI size distribution is determined by DLS (Malvern Instruments, UK).

In an embodiment, the PDI of ≤0.1 is measured by DLS using a Malvern Zetasizer series instrument, such as for example the Zetasizer Nano S, Zetasizer APS, Zetasizer μV or Zetasizer AT machines (Malvern Instruments, UK). In an embodiment, the PDI of ≤0.1 is measured by DLS using a Malvern Zetasizer Nano S machine. Exemplary conditions and system settings are described above in respect of determining mean particle size.

The requirement that the lipid vesicle particles have a mean particle size of ≤120 nm and a PDI of ≤0.1 means that it is possible that some lipid vesicle particles in a given population will have a particle size that is greater than 120 nm. This is acceptable so long as the mean particle size remains ≤120 nm and the PDI remains ≤0.1. As is shown in Example 2, lipid vesicle particles that are sized to meet these specifications are advantageous over non-sized lipid vesicle particles in obtaining a suitable dried lipid/therapeutic agent preparation for subsequent solubilization in a hydrophobic carrier (i.e. in obtaining a clear solution).

Lipid vesicle particles as encompassed herein, having a mean particle size of ≤120 nm and a PDI of ≤0.1, may be prepared and provided by any suitable means. In an embodiment, the lipid vesicle particles are prepared in a manner in which their size is controlled in order to achieve the mean particle size of ≤120 nm and a PDI of ≤0.1. In an embodiment, lipid vesicle particles are subjected to one or more sizing steps or protocols to achieve the mean particle size of ≤120 nm and a PDI of ≤0.1. In an embodiment, the lipid vesicle particles may be prepared and provided by any combination of controlling their size during manufacture, performing sizing steps and/or any other means available in the art.

In an embodiment, the lipid vesicle particles must be subjected to one or more active steps of sizing in order to achieve the mean particle size of ≤120 nm and a PDI of ≤0.1. In an embodiment, the sizing is performed by filter-extrusion whereby lipid vesicle particles are passed through a filter membrane or a series of filter membranes (e.g. polycarbonate membranes) of appropriate pore size.

As such, as used herein, “sized lipid vesicle particles” refers to lipid vesicle particles that have been prepared by a means in which their size is controlled to attain a mean particle size of ≤120 nm and a PDI of ≤0.1 and/or they are sized to meet the criteria of having a mean particle size of ≤120 nm and a PDI of ≤0.1. The skilled person will be well aware of techniques available for providing lipid vesicle particles having a mean particle size of ≤120 nm and a PDI of ≤0.1. Reference herein to “non-sized lipid vesicle particles” or a “non-sized lipid vesicle particle preparation” means that the lipid vesicle particles have not be subject to procedures that limit their size to meet the defined size criteria, and/or they do not have a mean particle size of ≤120 nm and a PDI of ≤0.1.

In an embodiment, the sized lipid vesicle particles may be prepared from a lipid precursor that naturally forms lipid vesicle particles of the required size. For example, and without limitation, the sized lipid vesicle particles may be prepared using Presome® (Nippon Fine Chemical, Japan). Presome® is a dry powder precursor made up of different lipid combinations. Presome® is supplied ready to be wetted in a suitable buffer to prepare liposomes. The liposomes formed from Presome® have an average particle size of about 93 nm, and sizing procedures (e.g. membrane extrusion, high pressure homogenization, etc.) can be used to achieve the required mean particle size of ≤120 nm and PDI of ≤0.1. In an embodiment, Presome® may for example be wetted in sodium acetate, pH 9.0±0.5 to form liposomes. In an embodiment, the Presome® bulk dry powder may be made from DOPC/cholesterol (10:1 (w/w)) or DOPC alone.

In another embodiment, standard procedures for preparing lipid vesicle particles of any size may be employed. For example, conventional liposome forming processes may be used, such as the hydration of solvent-solubilized lipids. Exemplary methods of preparing liposomes are discussed, for example, in Gregoriadis 1990; and Frezard 1999. After the lipid vesicle particles are prepared, the non-sized lipid vesicle particle preparation is subjected to a sizing procedure to obtain lipid vesicle particles having a mean particle size of ≤120 nm and a PDI of ≤0.1. There are various techniques available for sizing lipid vesicle particles (see e.g. Akbarzadeh 2013). For example, in an embodiment, the non-sized lipid vesicle particle preparation may be sized by high pressure homogenization (microfluidizers), sonication or membrane based extrusion.

In an embodiment, the sized lipid vesicle particles may be prepared by adding the lipids to a suitable solvent (e.g. sodium phosphate, 50 mM, pH 7.0), shaking and/or stirring the lipid mixture (e.g. at 300 RPM for about 1 hour) and using membrane based extrusion to obtain the sized lipid vesicle particles. Exemplary, non-limiting embodiments of membrane based extrusion include: (i) passing a non-sized lipid vesicle particle preparation 20-40 times through a 0.2 μm polycarbonate membrane, and then 10-20 times through a 0.1 μm polycarbonate membrane; or (ii) passing a non-sized lipid vesicle particle preparation 20-40 times through a 0.2 μm polycarbonate membrane, then 10-20 times through a 0.1 μm polycarbonate membrane, and then 10-20 times through a 0.08 μm polycarbonate membrane.

In a particular embodiment, the sizing may be performed by passing a non-sized lipid vesicle particle preparation 25 times through a 0.2 μm polycarbonate membrane, and then 10 times through a 0.1 μm polycarbonate membrane. In another particular embodiment, the sizing may be performed by passing a non-sized lipid vesicle particle preparation 25 times through a 0.2 μm polycarbonate membrane, then 10 times through a 0.1 μm polycarbonate membrane, and then 15 times through a 0.08 μm polycarbonate membrane.

In an embodiment of step (a), the sized lipid vesicle particles may be provided in a suitable solvent for mixing with the solubilized therapeutic agent. The solvent may be the same solvent that is used for solubilizing the therapeutic agent, or a different compatible solvent. By “compatible” it is meant that the solvent will not cause the solubilized therapeutic agent to come out of solution during the mixing of step (b). Alternatively, in another embodiment, the sized lipid vesicle particles may be provided in a dehydrated form and resuspended by adding the solution containing the solubilized therapeutic agent.

In accordance with the disclosed methods, in step (b) the sized lipid vesicle particles are mixed with at least one solubilized therapeutic agent to form a mixture.

As used herein, the term “therapeutic agent” is any molecule, substance or compound that is capable of providing a therapeutic activity, response or effect in the treatment or prevention of a disease, disorder or condition, including diagnostic and prophylactic agents. As described elsewhere herein, the term “therapeutic agent” does not include or encompass a T-helper epitope or an adjuvant, which are separately described in the present specification and are different components that may or may not be included in the methods, dried preparations, compositions, uses and kits disclosed herein.

In an embodiment, the therapeutic agent is a peptide antigen, a DNA or RNA polynucleotide that encodes a polypeptide (e.g. mRNA), a hormone, a cytokine, an allergen, a catalytic DNA (deoxyribozyme), a catalytic RNA (ribozyme), an antisense RNA, an interfering RNA (e.g. siRNA or miRNA), an antagomir, a small molecule drug, a biologic drug, an antibody, or a fragment or derivative of any one thereof; or a mixture thereof.

In an embodiment, the methods disclosed herein are for formulating a single type of therapeutic agent in a composition. In another embodiment, the methods disclosed herein are for formulating a mixture of multiple different therapeutic agents in a single composition. In an embodiment, the methods disclosed herein are for formulating a mixture of 2, 3, 4, 5, 6, 7, 8, 9, 10, 11, 12, 13, 14, 15, 16, 17, 18, 19, 20, 21, 22, 23, 24, 25, 26, 27, 28, 29, 30 or more different therapeutic agents in a single composition. In an embodiment, the methods disclosed herein are for formulating a mixture of 10-20 different therapeutic agents in a single composition.

In a particular embodiment, the therapeutic agent is one or more peptide antigens as described herein. In an embodiment, the peptide antigen is a synthetically produced polypeptide.

The peptide antigen may be a polypeptide of any length. In an embodiment, the peptide antigen may be 5 to 120 amino acids in length, 5 to 100 amino acids in length, 5 to 75 amino acids in length, 5 to 50 amino acids in length, or 5 to 30 amino acids in length. In an embodiment, the peptide antigen may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length.

In an embodiment, the peptide antigen is 20 to 30 amino acids in length. In an embodiment, the peptide antigen is 27 amino acids in length.

In an embodiment, the sized lipid vesicle particles are mixed with a single solubilized peptide antigen. In an embodiment, the sized lipid vesicle particles are mixed with up to 30 or more different solubilized peptide antigens

In an embodiment, the sized lipid vesicle particles are mixed with two or more different solubilized peptide antigens. In an embodiment, the sized lipid vesicle particles are mixed with 2, 3, 4, 5, 6, 7, 8, 9, 10, 11, 12, 13, 14, 15, 16, 17, 18, 19, 20, 21, 22, 23, 24, 25, 26, 27, 28, 29, 30 or more different solubilized peptide antigens. In an embodiment, the sized lipid vesicle particles are mixed with 2 to 30 different solubilized peptide antigens, 5 to 30 different solubilized peptide antigens, 10 to 20 different solubilized peptide antigens, or 10 to 15 different solubilized peptide antigens. In an embodiment, the sized lipid vesicle particles are mixed with five or more different solubilized peptide antigens. In an embodiment, the sized lipid vesicle particles are mixed with 10, 11, 12, 13, 14, 15, 16, 17, 18, 19 or 20 different solubilized peptide antigens.

In an embodiment, the one or more peptide antigens are neoantigens as described herein. In an embodiment, the methods disclosed herein are for formulating a mixture of multiple different neoantigens in a single composition. In an embodiment, the methods disclosed herein are for formulating a mixture of mixture of 5, 6, 7, 8, 9, 10, 11, 12, 13, 14, 15, 16, 17, 18, 19, 20, 21, 22, 23, 24, 25, 26, 27, 28, 29, 30 or more different neoantigens in a single composition. In an embodiment, the methods disclosed herein are for formulating a mixture of 10 to 20 different neoantigens in a single composition. In an embodiment, each of the different neoantigens is 20 to 30 amino acids in length.

Further exemplary embodiments of therapeutic agents that may be used in the practice of the methods disclosed herein are described below, without limitation.

The disclosed methods allow multiple different therapeutic agents to be formulated in a single pharmaceutical composition. In an embodiment, the therapeutic agents may have different characteristics relating to isoelectric point, solubility (e.g. co-solubility), stability and/or immunogenicity, and do not need to be pre-selected based on these characteristics for compatibility in the disclosed method. In a particular embodiment, the therapeutic agents are peptide antigens, such as neoantigens. As such, in an embodiment, the peptide antigens do not need to be subjected to extensive peptide selection steps to determine suitability for use in the disclosed methods or to determine compatibility with other peptide antigens (e.g. co-solubility). More particularly, in an embodiment, the peptide antigens do not need to be pre-selected based on any characteristic relating to isoelectric point, solubility, stability and/or immunogenicity.

As used herein, “isoelectric point” has its ordinary meaning in the art in that it refers to the pH at which a particular molecule (e.g. therapeutic agent) carries no net electrical charge. The isoelectric point (pI) value can affect the solubility of a molecule at a given pH. For example, amino acids that make up polypeptides/proteins may be positive, negative, neutral or polar in nature, and as a whole give a polypeptide/protein its overall charge. At a pH below their pI, polypeptides/proteins carry a net positive charge, whereas above their pI they carry a net negative charge. As such, pI is often an important consideration in the formulation of pharmaceutical compositions, such as peptide-based formulations. A number of algorithms for estimating isoelectric points have been developed, most of which use the Henderson-Hasselbalch equation with different pK values.

As used herein, “solubility” has its ordinary meaning in the art in that it refers to the ability of a substance to dissolve in a solvent. The solubility of the substance fundamentally depends on the physical and chemical properties of the substance and solvent as well as on temperature, pressure and the pH of the solution. Solubility may be measured in terms of the maximum amount of substance dissolved in a solvent at equilibrium.

As used herein, “stability” refers to the ability of a substance to remain unchanged over time under defined or reasonably expected conditions. Like solubility, the stability of a substance fundamentally depends on the physical and chemical properties of the substance and the environment in which it is placed. Stability may be measured in terms of degradation, aggregation and/or misfolding.

As used herein, “immunogenicity” refers to the ability of a particular substance, such as an antigen or epitope, to provoke an immune response in the body of a subject (e.g. human). In an embodiment, immunogenicity is the ability to induce a humoral (antibody) and/or cell-mediated (CTL) immune response. There are many in vitro and in vivo techniques known in the art to measure immunogenicity, including techniques involving enzyme linked immunosorbant assay (ELISA) or enzyme linked immunospot assay (ELISPOT).

As used herein, “characteristic relating to isoelectric point, solubility, stability and/or immunogenicity” refers any physical or chemical property of the therapeutic agent that may be relevant to its isoelectric point, solubility, stability and/or immunogenicity. For example, and without limitation, in respect of peptide antigens such characteristics may include peptide length, sequence, molecular weight, charge, polarity, hydrophobicity and/or hydrophilicity. These characteristics may be in respect of the peptide antigen as a whole or the amino acids of which they are comprised. Notably, it is contemplated that one or more of these characteristics may be observed or determined during the normal course of working with the therapeutic agents. However, an advantage of the methods disclosed herein is that this is not necessary to pre-determine these properties in order to prepare a suitable dried lipid/therapeutic agent preparation.

In an embodiment, the different solubilized therapeutic agents used in the methods disclosed herein have one or more different characteristics relating to isoelectric point, solubility, stability and/or immunogenicity.

In an embodiment, the different solubilized therapeutic agents used in the methods disclosed herein have a different length, sequence, molecular weight, charge, polarity, hydrophobicity and/or hydrophilicity. In instances where multiple different therapeutic agents are used in the methods disclosed herein, these differences may be in respect of a comparison between any one or more of the therapeutic agents, and the multiple therapeutic agents as a whole may have any combination of differences.

As used herein, by “solubilized therapeutic agent”, it is meant that the therapeutic agents are dissolved in a solvent. In an embodiment, this may be determined visually by the naked eye by observing a clear solution. A hazy solution is indicative of insolubility and is not desired for the methods disclosed herein as it may be problematic to forming a clear composition when the dried lipid/therapeutic agent preparation is subsequently solubilized in the hydrophobic carrier.

As described herein, the disclosed methods are advantageous in preparing stable, water-free compositions comprising lipids and therapeutic agents. To prepare such compositions, there are complex formulation requirements. The solvents used in the preparation the sized lipid vesicle particle/therapeutic agent mixture must not only be suitable for solubilizing the therapeutic agents in an aqueous environment with the lipids, but must also be suitable for forming a dried lipid/therapeutic agent preparation that will be compatible with a hydrophobic carrier (e.g. any salts and/or non-volatile solvents should preferably be compatible with the hydrophobic carrier). Moreover, in embodiments involving multiple therapeutic agents, the solvent(s) ideally would be suitable for solubilizing all of the therapeutic agents and mixing with the sized lipid vesicle particles.

Through extensive study, the present inventors have identified a number of exemplary solvents which may have universal application in the methods disclosed herein, including optimal salt and/or pH conditions for obtaining a clear pharmaceutical composition.

Exemplary solvents that may be used include, for example and without limitation, zwitterionic solvents. Non-limiting examples of zwitterionic solvents include HEPES (4-(2-hydroxyethyl)-1-piperazineethanesulfonic acid), MOPS (3-(N-Morpholino) propanesulfonic acid) and MES (2-(N-morpholino)ethanesulfonic acid).

In another embodiment, exemplary solvents are aqueous salt solutions. Salts provide useful properties in solubilizing the therapeutic agents, and it has also been recognized herein that certain salts provide stability to the dried lipid/therapeutic agent preparation. Non-limiting examples of such solvents include sodium acetate, sodium phosphate, sodium carbonate, sodium bicarbonate, potassium acetate, potassium phosphate, potassium carbonate, and potassium bicarbonate.

In an embodiment, the solvent is aqueous sodium acetate. It has been observed in the course of the present invention that sodium acetate imparts favourable properties to the dried lipid/therapeutic agent preparation for subsequent solubilization in the hydrophobic carrier. This is observed over a broad pH range (e.g. 6.0-10.5). For dissolution of multiple therapeutic agents (e.g. as a universal solvent), a molarity in the range of 50-200 mM may be preferred.

In an embodiment, the sodium acetate may be 25-250 mM sodium acetate having a pH in the range of 6.0-10.5. In an embodiment, the solvent is 100 mM sodium acetate having a pH of 6.0±1.0. In an embodiment, the solvent is 100 mM sodium acetate having a pH of 9.5±1.0.

In an embodiment, the solvent is 100 mM sodium acetate having a pH of 6.0.

In an embodiment, the solvent is 100 mM sodium acetate having a pH of 9.5.

In an embodiment, the solvent is aqueous sodium phosphate. It has been observed in the course of the present invention that sodium phosphate imparts favourable properties to the dried lipid/therapeutic agent preparation for subsequent solubilization in the hydrophobic carrier. More particularly, it has been observed that aqueous sodium phosphate exhibits favourable properties for dissolving multiple peptide antigens at lower molarity (i.e. ≤100 mM). For solubilization of the dried lipid/therapeutic agent preparation in the hydrophobic carrier, a pH in the range of 6.0-7.0 may be preferred.

In an embodiment, the sodium phosphate may be 25-250 mM sodium phosphate having a pH in the range of 6.0-8.0. In an embodiment, the solvent is 50 mM sodium phosphate having a pH of 7.0±1.0. In an embodiment, the solvent is 100 mM sodium phosphate having a pH of 6.0±1.0.

In an embodiment, the solvent is 50 mM sodium phosphate having a pH of 7.0.

In an embodiment, the solvent is 100 mM sodium phosphate having a pH of 6.0.

As encompassed herein, the therapeutic agents are either solubilized in a solvent prior to mixing with the sized lipid vesicle particles or the therapeutic agents are solubilized upon being mixed with the sized lipid vesicle particles. In this latter embodiment, the therapeutic agents may be added as a dry powder to a solution containing the sized lipid vesicle particles or both the sized lipid vesicle particles and dry therapeutic agents may be mixed together in a fresh solvent.

When the therapeutic agents are solubilized prior to mixing with the sized lipid vesicle particles, in embodiments where more than one therapeutic agent is used, the individual therapeutic agents may be solubilized together in the same solvent or separate from each other in different solvents. When three or more therapeutic agents are used, some of the agents may be solubilized together and others may be solubilized individually.

In an embodiment, all of the therapeutic agents are solubilized together in the same solvent as a therapeutic agent stock.

In embodiments of the disclosed methods in which multiple different therapeutic agents are to be formulated, it may be advantageous to initially solubilize the therapeutic agents in a mild/weak acidic solvent (for a mixture of predominantly basic therapeutic agents) or a mild/weak basic solvent (for a mixture of predominantly acidic therapeutic agents). Exemplary acidic solvents that may be used include, without limitation, hydrochloric acid, acetic acid. Exemplary basic solvents that may be used include, without limitation, sodium hydroxide, sodium bicarbonate, sodium acetate and sodium carbonate. For a mixture of predominantly neutral therapeutic agents, an exemplary solvent may be dimethyl sulfoxide (DMSO).

In the methods disclosed herein, the therapeutic agents may be solubilized in any of the solvents described herein. Based on the present disclosure, the skilled person could also identify other solvents that may be used that exhibit similar characteristics to those described herein.

As encompassed herein, any other optional components (e.g. T-helper epitope and/or adjuvant) may also be solubilized or mixed in the solvents described herein. Likewise, the sized lipid vesicle particles may be provided in the solvents described herein.

In an embodiment, at any stage of preparing the solubilized therapeutic agents or mixing the therapeutic agents with the sized lipid vesicle particles, one or more T-helper epitopes and/or adjuvants may be added. The adjuvant and T-helper epitope may be added at any stage and in any order, independent of one another. Typically, embodiments of the methods disclosed herein that involve the use of T-helper epitopes and/or adjuvants are those in which the therapeutic agent comprises at least one peptide antigen or a polynucleotide encoding an antigen.

Exemplary embodiments of T-helper epitopes and adjuvants that may be used in the practice of the methods disclosed herein are described below, without limitation. In an embodiment, the T-helper epitope comprises or consists of the modified Tetanus toxin peptide A16L (830 to 844; AQYIKANSKFIGITEL; SEQ ID NO: 1). In an embodiment, the adjuvant is a polyI:C nucleotide adjuvant.

In an embodiment, the mixing of step (b) may be performed using the same or different solvents as are used for preparing the sized lipid vesicle particles and/or for solubilizing the therapeutic agents, T-helper epitopes and/or adjuvants.

In an embodiment, the mixing of step (b) is performed in a sodium acetate or sodium phosphate solution.

In an embodiment, the mixing of step (b) is performed in 25-250 mM sodium acetate having a pH in the range of 6.0-10.5 or 25-250 mM sodium phosphate having a pH in the range of 6.0-8.0.

In an embodiment, the mixing of step (b) is performed in 50 mM sodium acetate having a pH of 6.0±1.0, 100 mM sodium acetate having a pH of 9.5±1.0, 50 mM sodium phosphate having a pH of 7.0±1.0 or 100 mM sodium phosphate having a pH of 6.0±1.0.

In an embodiment, the mixing of step (b) is performed in 50 mM sodium phosphate having a pH of 7.0, 100 mM sodium phosphate having a pH of 6.0, 50 mM sodium acetate having a pH of 6.0, or 100 mM sodium acetate having a pH of 9.5.

In an embodiment, after the sized lipid vesicle particles are mixed with the solubilized therapeutic agent and optionally other components (e.g. T-helper epitope and/or adjuvant), the pH of the mixture is adjusted to 10±1.0. In an embodiment, the pH is increased to 10±1.0 when it was sodium acetate that was used as the solvent.

The actual mixing of the sized lipid vesicle particles and the at least one solubilized therapeutic agent may be performed under any suitable conditions for obtaining a generally homogenous mixture of the sized lipid vesicle particles and the therapeutic agent. However, the mixing should not be performed under aggressive conditions that might cause the sized lipid vesicle particles and/or therapeutic agents to precipitate out of solution. In an embodiment, the mixing may be performed with gentle shaking or stirring at 100-500 RPM for a period of 2-60 minutes. In an embodiment, the mixing may be performed by shaking/stirring at 300 RPM for a period of about 3 minutes. In another embodiment, the mixing may be performed by shaking/stirring at 300 RPM for a period of about 15 minutes.

Irrespective of the manner in which the mixing is performed, in the methods disclosed herein the therapeutic agents are only to be mixed with sized lipid vesicle particles (i.e. lipid vesicle particles having a mean particle size of ≤120 nm and a PDI of ≤0.1). None of the therapeutic agents are to be added to the lipid vesicle particles prior to sizing, such as for example during formation of the lipid vesicle particles. When the therapeutic agents are added in step (b), the provided sized lipid vesicle particles contain no therapeutic agent. As described herein, this feature of the disclosed methods is relevant to obtaining a clear pharmaceutical composition.

In accordance with the disclosed methods, in step (c) the mixture of sized lipid vesicle particles and at least one solubilized therapeutic agent is dried to form a dried lipid/therapeutic agent preparation.

As used herein, the terms “dried preparation”, “dried lipid/therapeutic agent preparation” or “dried preparation comprising lipids and a therapeutic agent”, used interchangeably, do not necessarily mean that the preparation is completely dry. For example, depending on the solvent or solvents used in the methods disclosed herein, it is possible that a small component of volatile and/or non-volatile material will remain in the dried preparation.

In an embodiment, the non-volatile material will remain. By “dried preparation”, it is meant that the preparation no longer contains substantial quantities of water. The process used to dry the preparation should be capable of removing substantially all water from the sized lipid vesicle particle/therapeutic agent mixture. Thus, in an embodiment, the dried preparation is completely free of water. In another embodiment, the dried preparation may contain a residual moisture content based on the limitations of the drying process (e.g. lyophilization). This residual moisture content will typically be less than 2%, less than 1%, less than 0.5%, less than 0.25%, less than 0.1%, less than 0.05% or less by weight of the dried preparation. This residual moisture content will not be more than 5% by weight of the dried preparation as this would result in a product that is not clear.

Various methods may be used to dry the sized lipid vesicle particle/therapeutic agent mixture, which are known in the art. In an embodiment, the drying is performed by lyophilization, spray freeze-drying, or spray drying. The skilled person is well-aware of these drying techniques and how they may be performed.

In an embodiment, the drying is performed by lyophilization. As used herein, “lyophilization”, “lyophilized” and “freeze-drying” are used interchangeably. As is well known in the art, lyophilization works by freezing the material and then reducing the surrounding pressure to allow the volatile solvent (e.g. water) in the material to sublime directly from the solid phase to the gas phase.

Any conventional freeze-drying procedure may be used to carry out the drying step of the methods disclosed herein. In an embodiment, the lyophilization is performed by sequential steps of loading, freezing, evacuation and drying (e.g. primary drying and secondary drying).

In an embodiment, the lyophilization is performed according to the protocol set forth in Table 8 below (Example 4). Briefly, the mixture of sized lipid vesicle particles and therapeutic agent is frozen to a temperature of about −40° C. over a period of time of about 5 minutes. Evacuation is then performed by reducing the pressure to about 100 mT. The mixture is then dried. A primary drying is performed for about 1 hour by increasing the temperature to about −30° C. under the reduced pressure. Then, a secondary drying is performed for about 20 minutes by further increasing the temperature to about 25° C. under the reduced pressure.

Relevant considerations for the freezing and drying stages include:
Freezing: It is important to cool the material below its triple point, i.e. the lowest temperature at which the solid and liquid phases of the material can coexist. This ensures that sublimation rather than melting will occur in the following steps.
Primary Drying: Enough heat is supplied for sublimation to occur. This phase may be performed slowly (hours to days). If too much heat is added, the material's structure could be altered.
Secondary Drying: Aims to remove any unfrozen water molecules. The temperature is raised (usually above 0° C.) to break any physico-chemical interactions that have formed between the water molecules and the frozen material.

In an embodiment that may be well-suited to personalized medicine, but also may have broader application, we have found that lyophilization of the sized lipid vesicle particle/therapeutic agent mixture can be performed within a sealed bag in a benchtop freeze dryer. This may be particularly advantageous because it reduces the number of steps that must be performed in a sterile laboratory environment and allows for the rapid manufacture of smaller batch sizes. For example, after sterile filtration of the sized lipid vesicle particle/therapeutic agent mixture, aseptically filled vials containing the mixture can be loaded and sealed within a sterile bag under sterile conditions. These sterile, sealed units can then undergo lyophilization in an open laboratory (i.e. non-sterile environment) using a benchtop freeze dryer. By this method, it is also possible to perform the freeze-drying with multiple different sealed units in a single freeze dryer. This may reduce the cost and time of manufacture by avoiding expensive freeze-drying steps in sterile laboratory environments using large-scale freeze dryers. Also, multiple different small-scale batches of dried lipid/therapeutic agent preparation may be prepared simultaneously in separate sealed sterile bags.

Thus, in an embodiment, the lyophilization is performed by loading one or more containers comprising the mixture of step (b) into a bag, sealing the bag to form a sealed unit, and then lyophilizing the sealed unit in a freeze dryer. In an embodiment, a single sealed unit may be loaded into the freeze dryer for lyophilization. In another embodiment, multiple separate sealed units may be loaded into a single freeze dryer for lyophilization. In an embodiment, the freeze dryer may contain 1, 2, 3, 4, 5, 6, 7, 8, 9, 10 or more different sealed units for the lyophilization.

In embodiments in which multiple separate sealed units are loaded into a single freeze dryer, the sealed units may: (i) each contain the same sized lipid vesicle particle/therapeutic agent mixture as the other sealed units, (ii) each contain a different sized lipid vesicle particle/therapeutic agent mixture than the other sealed units, or (iii) any combination thereof (i.e. some of the sealed units may contain the same sized lipid vesicle particle/therapeutic agent mixture as other sealed units and some sealed units may contain a different sized lipid vesicle particle/therapeutic agent mixture). The difference between the sized lipid vesicle particle/therapeutic agent mixtures in the sealed units may be in respect of the lipids used to prepare the vesicle particles, the therapeutic agent(s) included in the mixture (e.g. different peptide antigens or neoantigens) and/or any other component. In a particular embodiment, it is the therapeutic agent(s) that is different as between the sealed units. For the manufacture of pharmaceutical grade compositions, each individual sealed unit should only comprise containers with the same sized lipid vesicle particle/therapeutic agent mixture.

For ease of handling, the containers may be loaded onto a tray and the tray then sealed within the bag. In an embodiment, the tray is a metal tray or a plastic tray.

The container comprising the sized lipid vesicle particle/therapeutic agent mixture may be any container suitable for lyophilization. In an embodiment, the container is a vial, bottle, flask, test tube or any suitable alternative. In an embodiment, the container is a vial, such as a glass or a plastic vial. In an embodiment, the vial is a glass vial. In an embodiment, the container is a 2 ml or 3 ml glass vial, such as for example a 2 ml or 3 ml 13 MM FTN BB LYO PF vial. The container may further comprise a stopper and/or a seal suitable for lyophilization. In an embodiment, the stopper is a vented stopper. In an embodiment, the stopper is a Fluorotec Lyophilization Closure, 13 MM, single vented stopper. In an embodiment, the seal is crimp seal, such as for example an aluminum crimp seal. In an embodiment, the seal is a West-Spectra Flip-Off 13 MM seal.

The bag containing the sample for lyophilization may be any bag that is suitable for lyophilization. In an embodiment, the bag should also be capable of being autoclaved to provide a sterile bag. To provide a sterile bag, the bag is autoclaved and subsequently maintained under sterile conditions. Thus, in an embodiment, the bag is a sterile, autoclaved bag.

In an embodiment, the bag is made of paper, plastic or a paper/plastic combination. In an embodiment, the paper is a medical-grade paper and the plastic is a polyester/polypropylene laminate film. Various types of bags suitable for sterilizing medical equipment are known in the art, and any of these bags may be used. In an embodiment, the sterile bag is a Fisherbrand™ Instant Sealing Sterilization Pouch (Fisher Scientific).

The lyophilization may be performed in any suitable freeze dryer. In an embodiment, the freeze dryer is a benchtop freeze dryer. In an embodiment, the freeze dryer is a Virtis benchtop lyophilizer. In an embodiment, the freeze dryer is in an open laboratory (i.e. non-sterile environment).

An advantageous property of the disclosed methods for preparing a dried lipid/therapeutic agent preparation is that by using sized lipid vesicle particles of the disclosed size limitations (i.e. ≤120 nm with a PDI of ≤0.1), the therapeutic agents are able to withstand (e.g. not precipitate out) each of the multiple different phases encountered during preparation of the pharmaceutical composition, i.e. aqueous phase, drying and hydrophobic phase. This is not observed with non-sized lipid vesicles, whereby despite adequate appearance of the dried preparation, a hazy solution with precipitate is observed for the final pharmaceutical composition.

Moreover, unexpectedly, by using the disclosed methods for preparing a dried lipid/therapeutic agent preparation, it was possible to generate a stable, clear, water-free pharmaceutical composition without any active step of encapsulating the therapeutic agent within the sized lipid vesicle particles. This is an advantageous property in that the disclosed methods avoid unnecessary processing steps which are costly, time-consuming and may ultimately result in loss of a significant quantity of therapeutic agent (e.g. any therapeutic agent not encapsulated in the lipid vesicle particles).

Without being bound by theory, subsequent to providing and mixing the sized lipid vesicle particles with the at least one solubilized therapeutic agent, it is believed that the sized lipid vesicle particles may rearrange to form different structures depending on the processing step (e.g. drying, solubilization in a hydrophobic carrier, etc.). The small and uniform size of the sized lipid vesicle particles (i.e. mean particle size ≤120 nm with a PDI ≤0.1) may make them particularly amenable to these conformation changes. For example, when placed in a hydrophobic carrier, the sized lipid vesicle particles may reorder to form alternate lipid-based structures as described herein. Indeed, it is believed that a rearrangement of the lipids occurs during these subsequent manufacturing steps, as shown for example by the SAXS analysis provided herein.

The methods disclosed herein for preparing a dried lipid/therapeutic agent preparation may further comprise a step of sterilization. Sterilization may be performed by any method known in the art. In an embodiment, the sterilization is performed by sterile filtration, steam heat sterilization, irradiation (e.g. gamma irradiation) or chemical sterilization. In a particular embodiment, the sterilization is performed by sterile filtration. In an embodiment, the sterile filtration may be performed between steps (b) and (c), i.e. after mixing the sized lipid vesicle particles with the therapeutic agent, but before drying.

Any conventional procedure for sterile filtration may be employed so long as it does not affect the solubility and stability of the therapeutic agents in the sized lipid vesicle particle mixture. In this regard, it may be desirable to perform the sterile filtration under low pressure conditions (e.g. between 30-50 psi).

The serial filtration may be performed using commercially available sterile filtration membranes (e.g. MilliporeSigma). In an embodiment, the sterile filtration is performed using a 0.22 μm-rated membrane, a 0.2 μm-rated membrane and/or a 0.1 μm-rated membrane. In an embodiment, the sterile filtration is performed by a single passage of the sized lipid vesicle particle/therapeutic agent mixture through a single filtration membrane. In another embodiment, the sterile filtration is performed by serially passing the sized lipid vesicle particle/therapeutic agent mixture sequentially through a combination of the same or different sized filtration membranes.

Without limitation, in an embodiment, the sterile filtration may be performed under the following conditions:

1) Filtration pressure: 30-50 psi nitrogen gas

2) Temperature: Room temperature

3) Product Contact Time: ≤45 minutes

4) Filter Type: Millipak-20 PVDF Filter, 0.22 μm

5) Size: 6 L batch size

In an embodiment, the sterile filtration is performed by passing the mixture of step (b) through a single Millipak-20 PVDF Filter, 0.22 μm. In another embodiment, the sterile filtration is performed by serially passing the mixture of step (b) through two or more sterile filtration membranes. In an embodiment of the serial sterile filtration, the mixture of step (b) is passed through two, three, four, five or more Millipak-20 PVDF 0.22 μm membranes. In an embodiment of the serial sterile filtration, the mixture of step (b) is passed through two Millipak-20 PVDF 0.22 μm membranes.

The methods disclosed herein for preparing a dried lipid/therapeutic agent preparation may further comprise a step of confirming that the sized lipid vesicle particles have retained a mean particle size of ≤120 nm and PDI of ≤0.1. As described elsewhere herein, there are several techniques, instruments and services that are available to measure the mean particle size and PDI of lipid vesicle particles, such as for example and without limitation TEM, SEM, AFM, FTIR, XPS, XRD, MALDI-TOF-MS, NMR and DLS.

In an embodiment, the step of confirming the size and PDI of the lipid vesicle particles is performed using a DLS ZETASIZER NANO-S particle size analyzer.

The step of size/PDI confirmation may be performed once or at multiple different times throughout the disclosed methods. In an embodiment, this step may be performed before the sized lipid vesicle particles are provided in step (a); before mixing the sized lipid vesicle particles with the therapeutic agent in step (b); after mixing the sized lipid vesicle particles with the therapeutic agent; and/or before performing the drying of step (c). In an embodiment, the size confirmation step is performed between steps (b) and (c) to confirm the size/PDI of the sized lipid vesicle particles before drying.

In an embodiment, the size confirmation step may be performed by analyzing a small sample volume of a preparation of interest. In another embodiment, the size confirmation step may be performed by analyzing a sample from a preparation that was prepared in parallel with a preparation of interest.

In an embodiment, the step of confirming the size/PDI of the sized lipid vesicle particles also comprises confirming the pH of the sized lipid vesicle particle/therapeutic agent preparation. In an embodiment, the pH is measured using the same machine that is used to measure the size/PDI of the sized lipid vesicle particles. In an embodiment, the pH is measured separately using any suitable device for determining pH. Exemplary solvents are discussed elsewhere herein and, in an embodiment, this step involves confirming that the solvent retains the desired pH as described herein. For example, in an embodiment where the sized lipid vesicle particles are suspended in sodium phosphate, this step involves confirming a pH of 6.0-8.0. In an embodiment where the sized lipid vesicle particles are suspended in sodium acetate, this step involves confirming a pH of 6.0-10.5. More specific exemplary pH values for these solvents, based on molarity, are described elsewhere herein.

The methods disclosed herein for preparing a dried lipid/therapeutic agent preparation may further comprise a step of evaluating the stability of the therapeutic agent(s) before and/or after the drying of step (c). The stability of the therapeutic agents may be measured by any known means or method. In embodiments in which a T-helper epitope is present, the methods disclosed herein may also comprise a step of evaluating the stability of the T-helper epitope before and/or after the drying of step (c).

The step of evaluating the stability of the therapeutic agent may be performed, for example and without limitation, by high-performance liquid chromatography (HPLC). HPLC is a technique which can be used to separate, identify and quantify each component in a mixture. Thus, by using HPLC it is possible to determine the approximate quantity of each therapeutic agent in a mixture, as well as characterize the therapeutic agent qualitatively (e.g. observe degradation products, dimerization products, etc.).

In an embodiment, the methods disclosed herein are capable of providing a sized lipid vesicle particle/therapeutic agent mixture in which at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% of the original amount/concentration of therapeutic agent is retained in undegraded form immediately before drying. In an embodiment, 100% of the original amount/concentration of therapeutic agent is retained in undegraded form immediately before drying.

In an embodiment, the methods disclosed herein are capable of providing a dried lipid/therapeutic agent preparation in which at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% of the original amount/concentration of therapeutic agent is retained in undegraded form immediately after drying. In an embodiment, 100% of the original amount/concentration of therapeutic agent is retained in undegraded form immediately after drying.

In an embodiment, the methods disclosed herein are capable of providing a dried lipid/therapeutic agent preparation in which at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% of the original amount/concentration of therapeutic agent is retained in undegraded form at least three months after drying. In an embodiment, 100% of the original amount/concentration of therapeutic agent is retained in undegraded form at least three months after drying.

In an embodiment, the methods disclosed herein are capable of providing a dried lipid/therapeutic agent preparation wherein one or more of the therapeutic agents show no degradation for up to 1, 2, 3, 4, 5, 6, 7, 8, 9, 10, 11, 12 months or more after drying. In a particular embodiment, the methods disclosed herein are capable of providing a dried lipid/therapeutic agent preparation wherein one or more of the therapeutic agents show no degradation for up to three months or more after drying.

In an embodiment of the methods disclosed herein, after step (b), each of the solubilized therapeutic agents is at a concentration of between about 0.1 mg/ml and 10 mg/ml. In an embodiment, each of the solubilized therapeutic agents is at a concentration of at least about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml or 1.0 mg/ml.

In an embodiment, the methods involve the use of five or more different therapeutic agents and after step (b), each of the different solubilized therapeutic agents is at a concentration of about 0.5 mg/ml.

In a particular embodiment of the methods disclosed herein, the therapeutic agents are peptide antigens. For example, the methods disclosed herein may be used in the preparation of peptide-based immunogenic compositions (e.g. vaccines).

Conventional vaccine strategies using whole organisms or large proteins have been highly efficacious for several decades, particularly in the treatment of infectious disease. However, the inclusion of unnecessary antigenic material is problematic in that it often gives rise to undesired reactivity, with protective immunity being dependent upon only a few select peptide epitopes within the formulation. This has created significant interest in peptide-based vaccines.

Fully synthetic peptide-based vaccines are the potential future of vaccination. Peptide vaccines rely on the usage of short peptide fragments to induce highly targeted immune responses. Peptide-based vaccines offer several advantages over conventional vaccines. For example, peptide antigens are less likely to induce undesired allergic or autoimmune responses due to the lack of unnecessary elements; chemical synthesis practically removes all of the problems associated with biological contamination; and peptides can be customized or multi-peptide approaches employed to target very specific objectives.

However, a drawback to peptide-based vaccination is that due to their relatively small size, peptide antigens are often weakly immunogenic and therefore typically require the assistance of adjuvants and/or an effective delivery system. Peptide antigens can also be difficult to formulate in pharmaceutical compositions, particularly when it is desired that the composition comprise multiple different peptide antigens. More particularly, in emerging areas of immunotherapy such as personalized medicine, difficulties in formulating suitable peptide-based compositions in a timely and cost-effective manner presents significant challenges.

Although the efficiency in identifying potential epitopes has vastly improved with the aid of sequencing techniques and the creation of computer algorithms (e.g. NetMHC which identify motifs predicted to bind MHC class I and/or MHC class II proteins), these technologies do little to accurately predict the ability to generate stable, immunologically effective compositions using peptide antigens. Moreover, while the use of multiple peptide antigens is often desirable to provide broader coverage through antigenic diversity, these types of vaccines present additional peptide selectivity requirements and are even more difficult to formulate as stable compositions, particularly in the context of specialized delivery systems that employ unique components, such as lipid-based delivery vehicles and/or hydrophobic carriers. Thus, despite advances, the formulation of suitable antigens has remained a crucial and time consuming step in the development of peptide-based vaccines. This has presented significant problems in fields of immunotherapy where the fast development of immunogenic compositions is crucial, such as personalized cancer vaccines.

In an embodiment, the present invention relates to advantageous methods for preparing dried peptide antigen preparations and pharmaceutical compositions comprising peptide antigens. The methods avoid time-consuming processing steps, and allow for the preparation of peptide-based antigen formulations without the need to perform extensive peptide selection steps. The disclosed methods allow multiple different peptide antigens, irrespective of their nature, to be formulated as a single pharmaceutical composition. Moreover, the method allows for the rapid production of a pharmaceutical composition containing one or more peptide antigens, which is expected to be particularly useful in the context of personalized cancer vaccines involving patient-specific neoantigens.

Thus, in an embodiment, the present disclosure relates to a method for preparing a dried peptide antigen preparation comprising the steps of: providing lipid vesicle particles having a mean particle size of ≤120 nm and a polydispersity index (PDI) of ≤0.1; mixing the lipid vesicle particles with one or more solubilized peptide antigen to form a mixture; and drying the mixture formed in step (b) to form a dried peptide antigen preparation.

In an embodiment of such methods, each of the one or more peptide antigens may be solubilized together to provide an antigen stock. Thus, in an embodiment of the methods disclosed herein, step (b) comprises: (b1) providing an antigen stock comprising the at least one solubilized peptide antigen and optionally a solubilized T-helper epitope; and (b2) mixing the antigen stock with the lipid vesicle particles to form the mixture.

In an embodiment, the antigen stock may be prepared by combining pre-solubilized antigens. In another embodiment, the antigen stock may be prepared by combining dry powder peptide antigens, adding a solvent and mixing the antigens in the solvent. In another embodiment, the antigen stock may be prepared by combining one or more dry powder peptide antigens with one or more pre-solubilized antigens.

The antigen stock may be prepared, for example, by combining dry powder peptide antigens in a single container, and solubilizing the peptide antigens with mixing and/or sonication in a solvent. In an embodiment, the dry powder mixture of peptide antigens may be solubilized and mixed by vortexing for 1-5 minutes and sonication for 1-5 minutes. In an embodiment, the dry powder mixture of peptide antigens may be solubilized and mixed by vortexing for about 1 minute and sonication for about 2 minutes.

In an embodiment, the T-helper epitope is added during preparation of an antigen stock by combining dry powder T-helper epitope with the dry powder mixture of antigen, and then solubilizing the mixture. In this embodiment, the T-helper epitope becomes part of the antigen stock to be mixed with the sized lipid vesicle particles. In another embodiment, the T-helper epitope may be mixed with the sized lipid vesicle particles separately from the peptide antigen. In this embodiment, the T-helper epitope may be added to the sized lipid vesicles particles before, after or at the same time as the peptide antigen. If multiple peptide antigens are used and they are mixed with the sized lipid vesicle particles separately, the T-helper epitope may be added in any order.

In an embodiment, an adjuvant is added during preparation of an antigen stock. It may be added by combining dry powder adjuvant with the dry powder mixture of antigen, and then solubilizing the mixture. Alternatively, it may be added to a solution of solubilized peptide antigen. In either of these embodiments, the adjuvant becomes part of the antigen stock to be mixed with the sized lipid vesicle particles. In another embodiment, the adjuvant may be mixed with the sized lipid vesicle particles separately from the peptide antigen. In this embodiment, the adjuvant may be added to the sized lipid vesicle particles before, after or at the same time as the peptide antigen. If multiple peptide antigens are used and they are mixed with the sized lipid vesicle particles separately, the adjuvant may be added in any order.

Irrespective of the order in which the peptide antigen, T-helper epitope and/or adjuvant may be combined, in the methods disclosed herein these components are only to be mixed with sized lipid vesicle particles. None of the peptide antigens (or therapeutic agents generally), T-helper and/or adjuvant are to be added to the lipid vesicle particles prior to sizing.

In an embodiment, an advantageous property of the methods disclosed herein is that they can be used to formulate multiple different therapeutic agents in a single composition.

An embodiment for achieving this purpose involves the preparation of an antigen stock comprising one or more of the solubilized peptide antigens, optionally together with a T-helper epitope.

In an embodiment, the antigen stock may be prepared as described above by dissolving a dry powder mixture of the peptide antigens and optionally a T-helper epitope in a solvent. In an embodiment, the solvent is 0.1 M sodium hydroxide solution (pH 12.0). In an embodiment, the antigens are each solubilized at a concentration of between about 0.5 mg/ml and 5.0 mg/ml. In an embodiment, the antigens are each solubilized at a concentration of between about 2.0 mg/ml. The antigen stock may then be added to an equal volume (1:1) of a sodium acetate or sodium phosphate solution, such as for example the solutions described herein. In an embodiment, the antigen stock may be added to an equal volume of 100 mM sodium acetate, pH 6.0. In an embodiment, the final concentration of each antigen in the antigen stock is about 1.0 mg/ml. This procedure is an exemplary embodiment of a universal approach that may be used to prepare an antigen stock containing multiple different peptide antigens. Given the disclosure herein, it would be within the ability of the skilled person to modify this procedure using different solvents based on the disclosure herein.

As disclosed herein, it was surprisingly found that peptide antigens could be added to pre-formed lipid vesicle particles having a mean particle size of ≤120 nm and a polydispersity index (PDI) of ≤0.1, and yet would still form a suitable dried antigen preparation for solubilization in a hydrophobic carrier. Without being bound by theory, it is believed that upon mixing with the antigen and/or during subsequent drying (e.g. lyophilization), the small uniformly sized lipid vesicle particles are capable of rearranging themselves (e.g. reordering and/or fusing). The rearrangement of the sized lipid vesicle particle structures may serve to effectively surround the peptide antigens in incompatible environments, e.g. hydrophobic peptides in an aqueous environment and then hydrophilic peptides in the hydrophobic carrier. In essence, it is believed that the sized lipid vesicle particles permit rearrangements that allow the peptide antigen payload to properly present to both the hydrophilic and hydrophobic environments. This was not observed with non-sized lipid vesicle particles whereby a hazy solution was ultimately obtained for the pharmaceutical composition.

As shown in Example 5 herein, HPLC analysis confirms that the methods provide a dried peptide antigen preparation in which the peptide antigens are stable, even under storage for lengthy periods of time (i.e. 3 months or longer). Example 5 provides an exemplary method for evaluating peptides by HPLC.

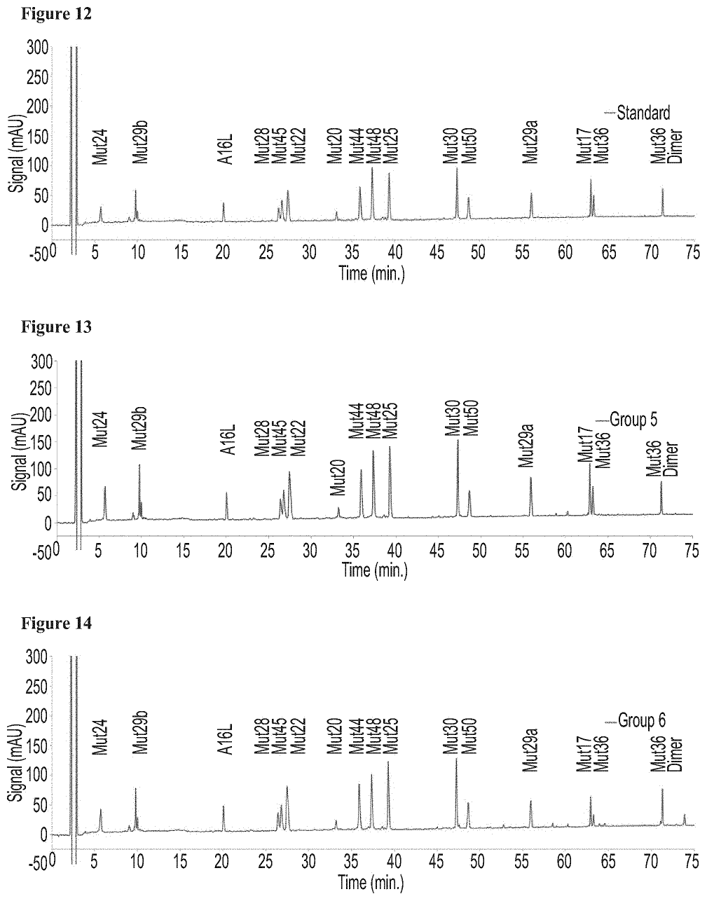

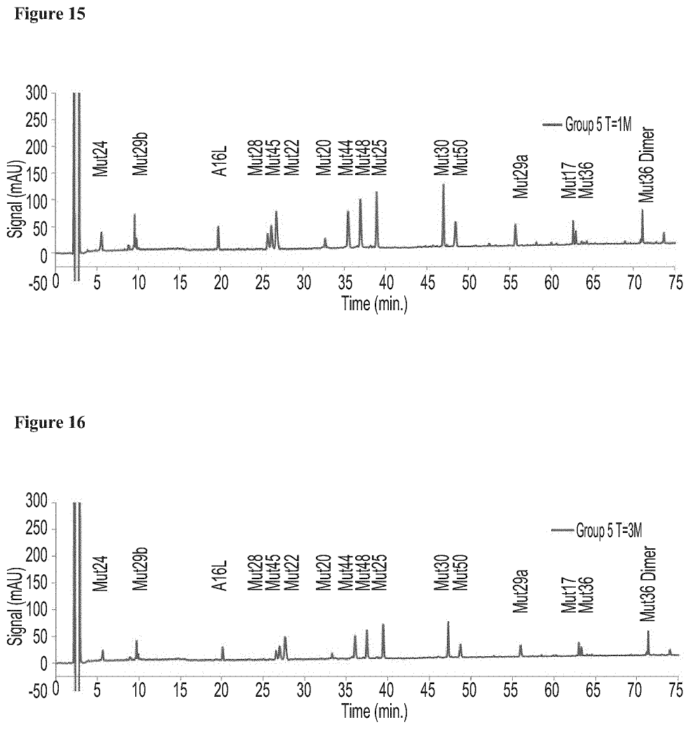

First, the peptides are characterized by performing HPLC analysis on a standard sample containing a known concentration of all the peptides (see e.g. FIG. 12 for an HPLC chromatogram showing the 14 neoantigens and A16L peptide of Example 5). Using this standard, HPLC analysis may then be used to quantify the peptides in the sized lipid vesicle particle/antigen mixture at a given time in the methods disclosed herein, such as for example before and/or after the drying of step (c). For example, chromatograms showing the 14 neoantigens before and after lyophilization are shown in FIGS. 13-16. The calculated peptide concentration is shown in Table 12 for immediately before freeze-drying, immediately after freeze-drying (T=0), 1 month after freeze-drying (T=1M) and 3 months after freeze-drying (T=3M). For the T=1M and T=3M time points, the samples were stored at −20° C. immediately after freeze-drying until the HPLC analysis was performed.

The peptide concentration for most of the peptides was within the set specifications at most of the time points. Based on the original protein concentration and the HPLC protocol used, the set specification for the neoantigens was 0.40-0.60 mg/mL and for the A16L peptide was 0.2-0.3 mg/mL. Mut36 was consistently outside the set specification; however, it is believed that this is due to the terminal cysteine residue leading to the formation of dimers. Indeed, as shown in the HPLC chromatograms, there is a separate peak observed for Mut36 dimers. Mut 29a and Mut17 also show values that are outside the set specification. These peptides contain internal cysteines which may result in the formation of dimers and therefore an inaccurate representation of peptide stability.

Thus, in an embodiment, the methods disclosed herein are capable of providing a sized lipid vesicle particle/antigen mixture wherein at least 80% of the original peptide concentration of each peptide antigen is retained in undegraded form immediately before drying.

In another embodiment, the methods disclosed herein are capable of providing a dried lipid/peptide antigen preparation wherein at least 75% of the original peptide concentration of each peptide antigen is retained in undegraded form immediately after drying.

In another embodiment, the methods disclosed herein are capable of providing a dried lipid/peptide antigen preparation wherein at least 70% of the original peptide concentration of each peptide antigen is retained in undegraded form at least for three months after drying.

In another embodiment, the methods disclosed herein are capable of providing a dried lipid/peptide antigen preparation wherein one or more of the peptide antigens show no degradation for up to 3 months after drying.

Overall, the HPLC analysis shows that the methods disclosed herein are capable of providing a dried lipid/peptide antigen preparation in which the peptide antigens are stable, even under storage for lengthy periods of time (i.e. 3 months or longer). Notably, since personalized neoantigen cancer vaccines typically have to be formulated within weeks to facilitate an effective immune response upon immunization, a shorter shelf-life for these products is expected to be required.

Moreover, the methods disclosed herein advantageously avoid the need to perform difficult size extrusion steps with larger peptide antigen-encapsulated liposomes. For pharmaceutical grade formulations, these size extrusion steps are often required to be performed on peptide antigen-encapsulated liposomal preparations in order to effectively perform sterile filtration. However, the high pressure (e.g. 5000 psi) under which these extrusion steps are performed can cause peptide antigens to come out of solution and bind to the filter. Surprisingly, it was found herein that these extrusion steps are not needed when peptide antigens are added to pre-formed lipid vesicle particles having a mean particle size of ≤120 nm and a PDI of ≤0.1. Even absent these intrusive extrusion steps, the dried antigen preparations prepared by the methods herein were able to form clear pharmaceutical compositions upon solubilization in a hydrophobic carrier.

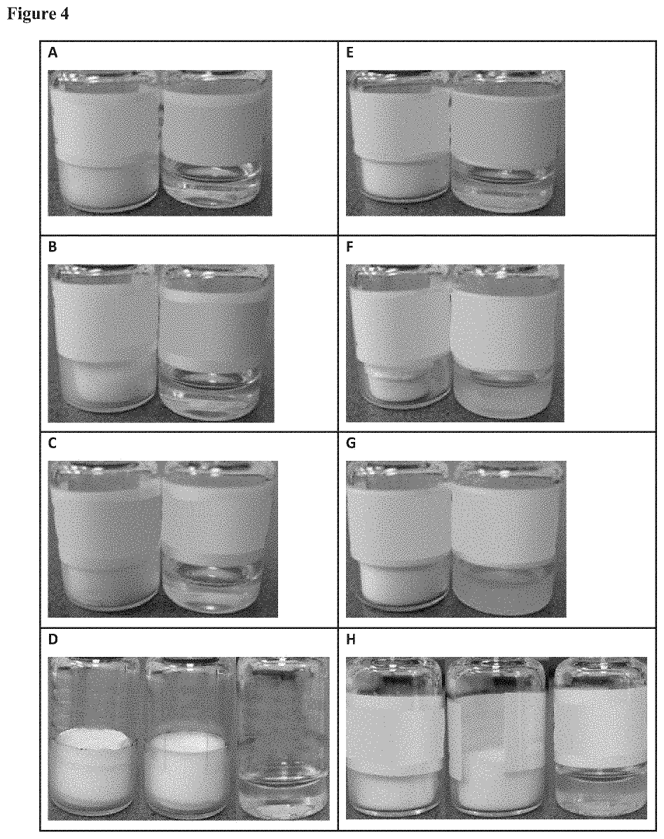

As shown in FIGS. 4A, 4B, 4C and 4D, a dried antigen preparation produced by the method disclosed herein is capable of generating a clear solution upon solubilization in a hydrophobic carrier (e.g. mineral oil). In contrast, when the dried antigen preparation is prepared with non-sized lipid vesicle particles, a hazy solution was formed upon solubilization (see FIGS. 4E, 4F, 4G and 4H).

In the pharmaceutical context, reproducibly obtaining a clear solution is an advantageous property. Pharmaceutical products must meet threshold requirements for regulatory approval, including homogeneity and reproducibility. The formation of precipitates and/or a lack of clarity of the solution are not desired properties as they may be indicative of a product in which the components (e.g. antigen) are not fully soluble. For a cloudy solution, additional processing steps may be required to establish homogeneity, and even then the composition may not be acceptable for pharmaceutical purposes. A slightly hazy solution may be acceptable as it may be the salts causing the haze, not precipitated antigen. However, a clear solution is preferred as it avoids the need to confirm that it is the salts precipitating out, and not the peptide antigen.

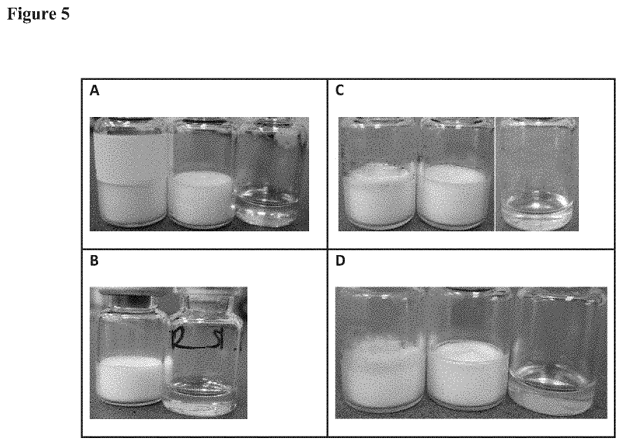

As shown herein, by using sized lipid vesicle particles, the disclosed methods form a clear solution upon solubilization in a hydrophobic carrier, whereas the non-sized lipid vesicle particle preparations do not. In fact, as shown in FIG. 5, the disclosed methods were capable of providing a clear solution irrespective of whether the sized lipid vesicle particles were made with DOPC/cholesterol or DOPC alone. These results indicate that the small lipid vesicle particles as used in the disclosed methods are stable even without cholesterol. This feature may be advantageous in the context of pharmaceutical compositions since fully synthetic products are often preferred, and it is not desirable to ingest cholesterol if it can be avoided.

In a further particular embodiment, the disclosed methods for producing a dried lipid/therapeutic agent preparation and pharmaceutical composition may also be particularly advantageous in the context of personalized cancer vaccines.

Personalized cancer vaccines are designed based on patient specific neoantigens, which are tumor specific antigens that arise via mutations (non-synonymous somatic mutations) altering amino acid coding sequences. Recently, with the aid of sequencing technologies, it has become possible to identify the mutations present within the protein-encoding part of the genome (the exome) of an individual tumor and thereby predict potential neoantigens. However, neoantigen-based personalized cancer vaccines typically have to be formulated within weeks to facilitate an effective immune response upon immunization. Timing is critical and poses significant hurdles. The time involved in first selecting potential neoantigens and then, within this population, selecting peptides having suitable characteristics to formulate stable compositions, has made progress in this field difficult at best.

In the methods disclosed herein for preparing a dried lipid/therapeutic agent preparation, since it is not necessary to prepare the sized lipid vesicle particles in the presence of the peptide antigen(s), the disclosed methods may reduce both the cost and time required to formulate personalized neoantigens. Time consuming processing steps can be avoided by preparing or purchasing stocks of sized lipid vesicle particles in advance.

Moreover, it was found that the disclosed methods were capable of formulating stable, water-free compositions with a large payload of different peptide antigens. As shown in Example 4, multiple different peptide antigens, irrespective of their nature, were able to be formulated together in one single composition using the disclosed methods. This was accomplished without the need to perform extensive antigen selection steps, such as in regard to isoelectric point, stability, solubility, immunogenicity and/or cross-compatibility between peptides (e.g. co-solubility).

It is a known manufacturing problem to formulate multiple different peptide antigens in a single composition. Since different peptide antigens will have different characteristics relating to their isoelectric point, stability and solubility, it would be expected that selection steps would be required to determine when in a manufacturing process, if at all, certain peptides could be formulated together. This is due in part to co-solubility issues whereby peptides having different characteristics will cause other peptides to come out of solution. This problematic feature is heightened in methods involving different phases of production (e.g. an aqueous phase and a hydrophobic phase). However, as shown in Example 4, the disclosed methods were able to formulate 14 different peptide antigens simultaneously in a single composition without any steps of peptide selection for isoelectric point, stability, solubility, and/or cross-compatibility. The peptides used are simply those identified by Castle et al. (2012) as potential neoantigens.

Moreover, in contrast to water-in-oil emulsions where antigen can be contained in water droplets formed in the oil, water-free compositions require the incorporation of all of the components (e.g. therapeutic agent, adjuvant, etc.) directly into the oil. It was unexpected that this could be done for even one peptide antigen by adding it outside of pre-formed sized lipid vesicle particle, let alone for 14 different peptide antigens each having distinct amino acid sequences (Table 12; FIGS. 13-16).

Despite these obstacles, by using the methods disclosed herein, we were able to formulate a stable, water-free composition comprising multiple different peptide antigens. This was achieved by using sized lipid vesicle articles to prepare a favourable dried antigen preparation for solubilization in a hydrophobic carrier. Without being bound by theory, it is believed that the ability to stably formulate so many peptides in a water-free formulation may be due to the unique rearrangement of the sized lipid vesicle particles during the method processing steps disclosed herein.

The ability of the methods disclosed herein to prepare a stable, water-free pharmaceutical composition comprising multiple different peptide antigens may have significant advantages in the field of personalized cancer vaccines.

To be successful in providing effective immunotherapy with personalized neoantigen-based cancer vaccines, it is important that the vaccine can be formulated and delivered to the patient within weeks. Given these time constraints, there is often little or no time for extensive peptide selection to identify the so-called “best” neoantigen in relation to characteristics such as isoelectric point, stability, solubility and/or immunogenicity. With the aim of providing suitable treatments, neoantigen vaccines currently being explored in clinical trials deliver 10-30 individual peptides in multiple formulations. While this approach reduces the time involved in selecting the most appropriate neoantigen, there remain significant issues with respect to formulation stability and/or immunogenicity, cost and patient compliance. Ideally, the antigens could be delivered in a single, stable formulation without time-consuming efforts in selecting antigens that have a particular set of characteristics relating to isoelectric point, stability, solubility (e.g. co-solubility) and/or immunogenicity.

The methods disclosed herein may offer advantages in that a multitude of different peptide antigens can be formulated in a single composition without time-consuming antigen selectivity steps. The dried antigen preparation disclosed herein can be rapidly solubilized in a hydrophobic carrier (e.g. oil) and administered easily by subcutaneous injection, saving time by avoiding complex formulation preparations for each peptide and avoiding multiple injections.

The disclosed methods are not only advantageous in the ability to formulate a number of different peptide antigens together, but also in the quantity of peptide antigen in the dried lipid/peptide antigen preparation and resulting pharmaceutical composition. As shown in Example 4, by the methods disclosed herein the dried lipid/peptide antigen preparation was capable of being solubilized in a hydrophobic carrier to provide a final composition comprising 7.0 mg/ml of peptide antigen. In addition, the composition also contains 0.25 mg/ml of T-helper peptide epitope.

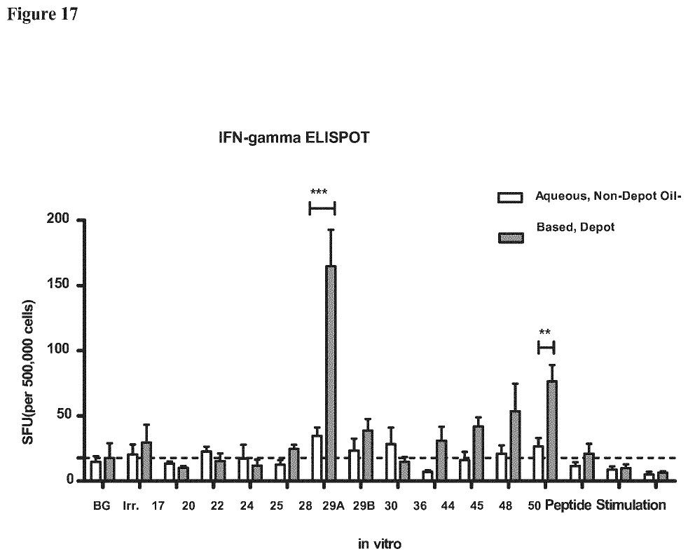

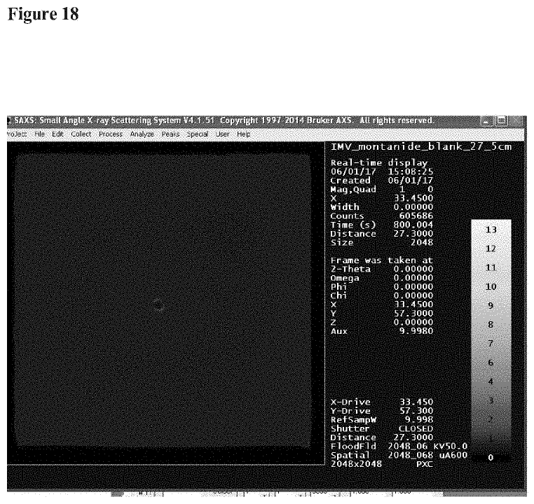

Moreover, the results disclosed herein show that, even with a large peptide antigen payload, the compositions retain the ability to promote rapid and enhanced immune responses after a single administration. As shown in FIG. 17, a composition prepared by the disclosed methods with 14 different neoantigens was able to provide enhanced CTL immune responses over a comparative aqueous composition. The immune response was improved by about 5-fold for neoantigen Mut25 and by about 3-fold for neoantigen Mut44. That a statistically significant difference was not observed for the other neoantigens is not surprising given that the antigens were not pre-selected for strong immunogenicity characteristics. Rather, as is common with personalized neoantigen vaccines, the goal is to include several different neoantigens with the hope that one or more will provide effective immunotherapy. As described herein, the methods and compositions of the present invention are believed to be well-suited and advantageous for this purpose.

Method for Preparing a Pharmaceutical Composition

In an embodiment, the present invention relates to a method for preparing a pharmaceutical composition. In an embodiment, the pharmaceutical composition is prepared by first preparing a dried lipid/therapeutic agent preparation according to the methods disclosed herein, and then solubilizing the dried preparation in a hydrophobic carrier.

As used herein, by “solubilizing” it is meant that the dried lipid/therapeutic agent preparation is restored to a liquid state by dissolving the dried constituents in a hydrophobic carrier. The hydrophobic carrier may be added by any means that will dissolve the dried constituents (e.g. the lipid and therapeutic agent) in the hydrophobic carrier. For example, and without limitation, the dried lipid/therapeutic agent preparation may be solubilized in the hydrophobic carrier by mixing of the two together. In an embodiment, solubilizing involves adding the hydrophobic carrier to the dried lipid/therapeutic agent preparation, allowing it to sit for 1-30 minutes, and then gently shaking or mixing the mixture for 1-15 minutes. This process can be repeated until the dried constituents are dissolved in the hydrophobic carrier (e.g. a clear solution is obtained).

In an embodiment, solubilizing involves adding the hydrophobic carrier to the dried lipid/therapeutic agent preparation, allowing it to sit for 5 minutes, and then gently shaking or mixing for 1 minute. This process can be repeated until the dried constituents are dissolved in the hydrophobic carrier (e.g. a clear solution is obtained).

As shown in FIGS. 4A, 4B, 4C and 4D, a dried lipid/therapeutic agent preparation produced by the methods disclosed herein is capable of generating a clear solution upon solubilization in a hydrophobic carrier. In contrast, when the dried lipid/therapeutic agent preparation is prepared with non-sized lipid vesicle particles, a hazy solution was formed (see FIGS. 4E, 4F, 4G and 4H).

As discussed herein, in the pharmaceutical context, reproducibly obtaining a clear solution is an advantageous property. Pharmaceutical products must meet threshold requirements for regulatory approval, including homogeneity and reproducibility. The formation of precipitates and/or a lack of clarity of the solution are not desired properties as they may be indicative of a product in which the components (e.g. therapeutic agent) are not fully soluble. For a cloudy solution, additional processing steps may be required to establish homogeneity, and even then the composition may not be acceptable for pharmaceutical purposes. A slightly hazy solution may be acceptable if it is the salts causing the haze, not precipitated therapeutic agent. However, a clear solution is advantageous.

By using sized lipid vesicle particles, the disclosed methods form a clear solution upon solubilization in a hydrophobic carrier, whereas the non-sized lipid vesicle particle preparations do not. In fact, as shown in FIG. 5, the disclosed methods were capable of providing a clear solution irrespective of whether the sized lipid vesicle particles were made with DOPC/cholesterol or DOPC alone. These results indicate that the sized lipid vesicle particles as used in the disclosed methods are stable even without cholesterol. This feature may be advantageous in the context of pharmaceutical compositions since fully synthetic products are often preferred, and it is not desirable to ingest cholesterol if it can be avoided.

In an embodiment, the step of solubilizing the dried lipid/therapeutic agent in a hydrophobic carrier results in a composition in which the dried constituents are fully dissolved in the hydrophobic carrier. In an embodiment, the dried constituents may not be completely dissolved in the hydrophobic carrier, but they are dissolved to a sufficient degree to reproducibly provide a clear solution.

As used herein, a “hydrophobic carrier” refers to a liquid hydrophobic substance. The term “hydrophobic carrier” may be referred to herein interchangeably as an “oil-based carrier”.

The hydrophobic carrier may be an essentially pure hydrophobic substance or a mixture of hydrophobic substances. Hydrophobic substances that are useful in the methods and compositions described herein are those that are pharmaceutically and/or immunologically acceptable. The carrier is typically a liquid at room temperature (e.g. about 18-25° C.), but certain hydrophobic substances that are not liquids at room temperature may be liquefied, for example by warming, and may also be useful.

Oil or a mixture of oils is a particularly suitable carrier for use in the methods and compositions disclosed herein. Oils should be pharmaceutically and/or immunologically acceptable. Suitable oils include, for example, mineral oils (especially light or low viscosity mineral oil such as Drakeol® 6VR), vegetable oils (e.g., soybean oil such as MS80), nut oils (e.g., peanut oil), or mixtures thereof. Thus, in an embodiment the hydrophobic carrier is a hydrophobic substance such as vegetable oil, nut oil or mineral oil. Animal fats and artificial hydrophobic polymeric materials, particularly those that are liquid at atmospheric temperature or that can be liquefied relatively easily, may also be used.

In some embodiments, the hydrophobic carrier may be, or comprise, Incomplete Freund's Adjuvant (IFA), a mineral oil-based model hydrophobic carrier. In another embodiment, the hydrophobic carrier may be, or comprise, a mannide oleate in mineral oil solution, such as that commercially available as Montanide® ISA 51 (SEPPIC, France). While these carriers are commonly used to prepare water-in-oil emulsions, the present disclosure relates to water-free compositions. As such, these carriers are not emulsified with water in the methods and compositions disclosed herein.

In an embodiment, the hydrophobic carrier is mineral oil or a mannide oleate in mineral oil solution.

In an embodiment, the hydrophobic carrier is Montanide® ISA 51.

In an embodiment, the hydrophobic carrier is MS80 oil, which is a mixture of mineral oil (Sigma Aldrich) and Span80 (Fluka). The components can be purchased separately and mixed prior to use.

In an embodiment, the present disclosure relates to a pharmaceutical composition prepared by the methods disclosed herein.

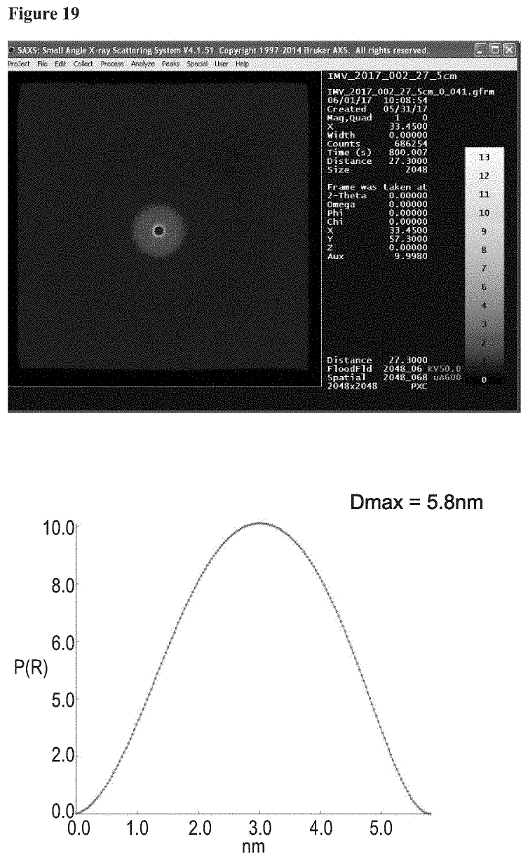

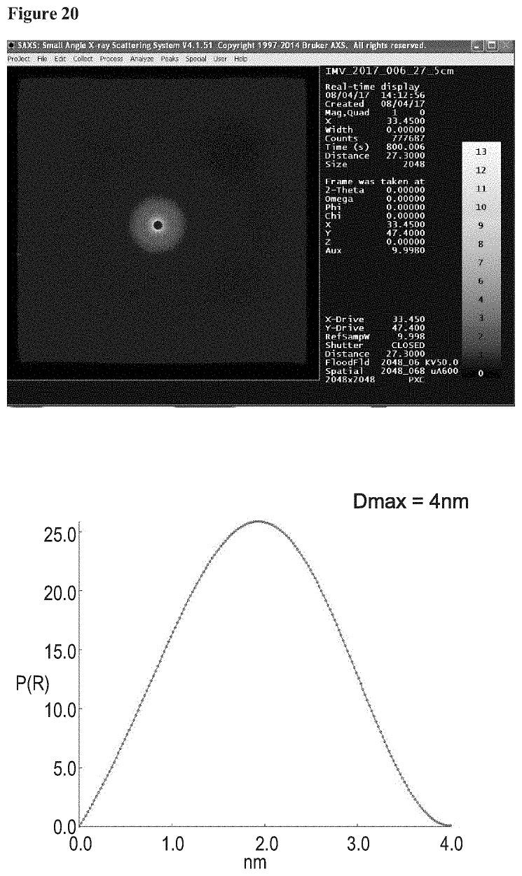

Small angle X-ray scattering (SAXS) can be used for the determination of the nanoscale structure of particle systems in terms of such parameters as averaged particle sizes, shapes, distribution and surface-to-volume ratio. Using the disclosed methods of preparing the dried lipid/therapeutic agent preparation with sized lipid vesicle particles, it has been found that in the hydrophobic carrier the lipids rearrange to form lipid-based structures having a single layer lipid assembly. This is shown in the SAXS patterns of FIGS. 19 and 20.

By “single layer lipid assembly”, it is meant that the lipids form aggregate structures in which the hydrophobic part of the lipids is oriented outwards toward the hydrophobic carrier and the hydrophilic part of the lipids aggregate as a core in the middle. From the SAXS patterns it is not possible to determine if the hydrophilic parts form a continuous single layer membrane (e.g. reverse micelle) or whether the core is a discontinuous aggregate. Irrespective of the configuration, the lipid-based structures comprise a single layer of lipids as opposed to a bilayer that would be found, for example, in liposomes. It is believed that in this configuration, hydrophilic therapeutic agents are in the core of the single layer lipid assembly and the hydrophobic therapeutic agents are solubilized in the non-polar oil.

Without being bound to theory, it is believed based on the examples herein that sizing of the lipid vesicle particles to attain a mean particle size of ≤120 nm and a PDI of ≤0.1 provides the dried lipid/therapeutic agent preparation with favourable properties that allows for better compatibility of the dried lipid/therapeutic agent preparation with the hydrophobic carrier. For example, the sized lipid vesicle particles may allow for an easier rearrangement of the lipid vesicle particles into the lipid-based structures upon solubilization in a hydrophobic carrier, thereby providing a clear product. This is perhaps due to the small, uniform size of the sized lipid vesicle particles. It is also believed that this property allows for the greater payload to be stably formulated in the hydrophobic carrier.

Pharmaceutical Compositions

In an embodiment, the present disclosure relates to a stable, water-free pharmaceutical composition comprising one or more lipid-based structures having a single layer lipid assembly, at least one therapeutic agent, and a hydrophobic carrier. Each of these components is individually described elsewhere herein in greater detail.

As used herein, the terms “pharmaceutical composition”, “composition”, “vaccine composition” or “vaccine” may be used interchangeably, as the context requires.

A pharmaceutical composition as disclosed herein may be administered to a subject in a therapeutically effect amount. As used herein, a “therapeutically effective amount” means an amount of the composition or therapeutic agent effective to provide a therapeutic, prophylactic or diagnostic benefit to a subject, and/or to stimulate, induce, maintain, boost or enhance an immune response in a subject. In some embodiments, a therapeutically effective amount of the composition is an amount capable of inducing a clinical response in a subject in the treatment of a particular disease or disorder. Determination of a therapeutically effective amount of the composition is well within the capability of those skilled in the art, especially in light of the disclosure provided herein. The therapeutically effective amount may vary according to a variety of factors such as the subject's condition, weight, sex and age.

The pharmaceutical compositions disclosed herein are water-free. As used herein, “water-free” means completely or substantially free of water, i.e. the pharmaceutical compositions are not emulsions.

By “completely free of water” it is meant that the compositions contain no water at all. In contrast, the term “substantially free of water” is intended to encompass embodiments where the hydrophobic carrier may still contain small quantities of water, provided that the water is present in the non-continuous phase of the carrier. For example, individual components of the composition may have small quantities of bound water that may not be completely removed by processes such as lyophilization or evaporation and certain hydrophobic carriers may contain small amounts of water dissolved therein. Generally, compositions as disclosed herein that are “substantially free of water” contain, for example, less than about 5%, 4%, 3%, 2%, 1%, 0.5%, 0.1%, 0.05% or 0.01% water on a weight/weight basis of the total weight of the carrier component of the composition. The compositions that still contain small quantities of water do not contain a sufficient amount of water such that an emulsion would be formed.

The pharmaceutical compositions disclosed herein are stable. By “stable”, it is meant that the lipids and therapeutic agents remain in solubilized form in the hydrophobic carrier. This is an advantageous property of the disclosed compositions. For example, in the context of embodiments involving multiple therapeutic agents (e.g. peptide antigens), it is possible to formulate a large variety of therapeutic agents together without pre-selection based on isoelectric point, solubility, stability and/or immunogenicity. The compositions are resistant to co-solubility issues that typically arise when therapeutic agents having different characteristics (e.g. length, sequence molecular weight, charge, polarity, hydrophobicity and/or hydrophilicity) are formulated together.

In an embodiment, the stability of the compositions may be based on the ability to prepare formulations that are a clear or slightly hazy solution. In an embodiment, the stability of the compositions may be based on the ability to prepare formulations that are a clear solution. By “clear solution”, it is meant that the solution does not have a cloudy or hazy appearance. In an embodiment, this may be determined visually by the naked eye by observing a clear solution or by measurement using a spectrophotometer. In an embodiment, the compositions may be visually inspected according to the European Pharmacopoeia (Ph. Eur.), 9^{th} edition, Section 2.9.20.

In an embodiment, the stability of the compositions may be based on the ability to prepare formulations that have no visible precipitates. By “visible precipitate”, it is meant to refer to precipitates that are either located on the wall of the container holding the composition or in the solution of the composition. In an embodiment, this may be determined visually by the naked eye by observing the absence of precipitates or by measurement using a spectrophotometer. In an embodiment, the compositions may be visually inspected according to the European Pharmacopoeia (Ph. Eur.), 9^{th} edition, Section 2.9.20.

In an embodiment, the stability of the compositions may be based on the observed stability of the therapeutic agents in the dried lipid/therapeutic agent preparation. For example, the stability of the compositions may be based on a substantially consistent therapeutic agent concentration over periods of storage for 1 week, 2 weeks, 3 weeks, 4 weeks, 5 weeks, 6 weeks, 7 weeks, 8 weeks, 9 weeks, 10 weeks, 11 weeks, 12 weeks, or longer. In an embodiment, the therapeutic agent concentration may be determined by HPLC analysis as described elsewhere herein. Stability of the therapeutic agents in the dried preparation is indicative their ability to be stably solubilized in the hydrophobic carrier.

In an embodiment, the stability of the compositions may further be evaluated by taking into consideration one or more of the following: identification and quantification of the therapeutic agents and/or the lipids, including identification of impurities and/or degradants (e.g. by RP-HPLC), particle size of the lipid-based structures having a single layer lipid assembly (e.g. by SAXS); optical density, viscosity (e.g. as per Ph.Eur. 2.2.9); extractable volume, such as from a syringe (e.g. as per Ph.Eur. 2.9.17), and immunogenicity assays (e.g. ELISpot).

In an embodiment, the compositions disclosed herein are stable for a period of at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 18 hours, at least 24 hours, at least 36 hours, at least 48 hours or longer, after solubilization in the hydrophobic carrier.

As described above, the pharmaceutical compositions disclosed herein comprise one or more lipid-based structures having a single layer lipid assembly. As used herein, the term “lipid-based structure” refers to any structure formed by lipids. The lipids that form the lipid-based structures having a single layer lipid assembly are the same lipids as described herein that form the sized lipid vesicle particles.

There are various lipid-based structures which may form, and the compositions disclosed herein may comprise a single type of lipid-based structure having a single layer lipid assembly or comprise a mixture of different lipid-based structures.

In an embodiment, the lipid-based structure having a single layer lipid assembly partially or completely surrounds the therapeutic agent. As an example, the lipid-based structure may be a closed vesicular structure surrounding the therapeutic agent. In an embodiment, the hydrophobic part of the lipids in the vesicular structure is oriented outwards toward the hydrophobic carrier.

As another example, the one or more lipid-based structures having a single layer lipid assembly may comprise aggregates of lipids with the hydrophobic part of the lipids oriented outwards toward the hydrophobic carrier and the hydrophilic part of the lipids aggregating as a core. These structures do not necessarily form a continuous lipid layer membrane. In an embodiment, they are an aggregate of monomeric lipids.

In an embodiment, the one or more lipid-based structures having a single layer lipid assembly comprise reverse micelles. A typical micelle in aqueous solution forms an aggregate with the hydrophilic parts in contact with the surrounding aqueous solution, sequestering the hydrophobic parts in the micelle center. In contrast, in a hydrophobic carrier, an inverse/reverse micelle forms with the hydrophobic parts in contact with the surrounding hydrophobic solution, sequestering the hydrophilic parts in the micelle center. A spherical reverse micelle can package a therapeutic agent with hydrophilic affinity within its core (i.e. internal environment).

Without limitation, the size of the lipid-based structures having a single layer lipid assembly is in the range of from 2 nm (20 A) to 20 nm (200 A) in diameter. In an embodiment, the size of the lipid-based structures having a single layer lipid assembly is between about 2 nm to about 10 nm in diameter. In an embodiment, the size of the lipid-based structures having a single layer lipid assembly is about 2 nm, 3 nm, 4 nm, 5 nm, 6 nm, about 7 nm, about 8 nm, about 9 nm, or about 10 nm in diameter. In an embodiment, the maximum diameter of the lipid-based structures is about 4 nm or about 6 nm. In an embodiment, the lipid-based structures of these sizes are reverse micelles.

In an embodiment, one or more of the therapeutic agents are inside the lipid-based structures after solubilization in the hydrophobic carrier. By “inside the lipid-based structure”, it is meant that the therapeutic agent is substantially surrounded by the lipids such that the hydrophilic components of the therapeutic agent are not exposed to the hydrophobic carrier. In an embodiment, the therapeutic agent inside the lipid-based structure is predominantly hydrophilic.

In an embodiment, one or more of the therapeutic agents are outside the lipid-based structures after solubilization in the hydrophobic carrier. By “outside the lipid-based structure”, it is meant that the therapeutic agent is not sequestered within the environment internal to the single layer lipid assembly. In an embodiment, the therapeutic agent outside the lipid-based structure is predominantly hydrophobic.

The pharmaceutical compositions disclosed herein comprise at least one therapeutic agent. Exemplary therapeutic agents are described elsewhere herein, without limitation.

In an embodiment, the therapeutic agent is a peptide antigen, a DNA or RNA polynucleotide that encodes a polypeptide (e.g. mRNA), a hormone, a cytokine, an allergen, a catalytic DNA (deoxyribozyme), a catalytic RNA (ribozyme), an antisense RNA, an interfering RNA (e.g. siRNA or miRNA), an antagomir, a small molecule drug, a biologic drug, an antibody, or a fragment or derivative of any one thereof; or a mixture thereof.

In a particular embodiment, the therapeutic agent is one or more peptide antigens. As used herein, the term “peptide antigen” is an antigen that is a protein or a polypeptide. Exemplary embodiments of peptide antigens that may be used in the compositions are described herein, without limitation.

In an embodiment, the composition comprises a single peptide antigen. In an embodiment, the composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more different peptide antigens. In an embodiment, the composition comprises 5 to 30 different peptide antigens, 10 to 20 different peptide antigens, or 10 to 15 different peptide antigens. In an embodiment, the composition comprises 10, 11, 12, 13, 14, 15, 16, 17, 18, 19 or 20 different peptide antigens. In an embodiment, the composition comprises 14 different peptide antigens.

By “different” peptide antigens, it is meant that none of the peptide antigens in the pharmaceutical composition have an identical amino acid sequence. The antigens may be derived from the same source (e.g. a virus, bacterium, protozoan, cancer cell, etc.) or from the same protein, but they do not share the same sequence.

In an embodiment, each of the peptide antigens in the composition may, independently, be 5 to 120 amino acids in length, 5 to 100 amino acids in length, 5 to 75 amino acids in length, 5 to 50 amino acids in length, or 5 to 30 amino acids in length. In an embodiment, the peptide antigens in the composition may, independently, be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length. In an embodiment, the peptide antigens in the composition may, independently, be 20 to 30 amino acids in length. Each of the peptide antigens may be the same or different lengths, or any combination thereof. In an embodiment, the peptide antigens are all 27 amino acids in length.

In an embodiment, the one or more peptide antigens are derived from human papillomavirus (HPV), human immunodeficiency virus (HIV), respiratory syncytial virus (RSV), Bacillus anthracis, Plasmodium and/or a survivin polypeptide.

In an embodiment, the one or more of the peptide antigens are derived from RSV, such as for example NKLCEYNVFHNKTFELPRARVNT (SEQ ID NO: 2) and/or NKLSEHKTFCNKTLEQGQMYQINT (SEQ ID NO: 3).

In an embodiment, the one or more of the peptide antigens in the composition are cancer-associated peptide antigens. In an embodiment, all of the peptide antigens in the composition are cancer-associated peptide antigens. Exemplary embodiments of cancer-associated peptide antigens that may be used in the compositions disclosed herein are described below, without limitation. In an embodiment, the cancer-associated peptide antigens may be one or more survivin antigens, such as for example and without limitation, those described herein.

In an embodiment, the one or more peptide antigens are FTELTLGEF (SEQ ID NO: 4), LMLGEFLKL (SEQ ID NO: 5), RISTFKNWPK (SEQ ID NO: 6), STFKNWPFL (SEQ ID NO: 7) or LPPAWQPFL (SEQ ID NO: 8); or any combination thereof. In an embodiment, the composition comprises all five of these peptide antigens (SEQ ID NOs: 4 to 7).

In an embodiment, the one or more of the peptide antigens in the composition are neoantigens. In an embodiment, all of the peptide antigens in the composition are neoantigens. Exemplary embodiments of neoantigens that may be used in the compositions disclosed herein are described below, without limitation. In an embodiment, the composition is one that comprises multiple neoantigens, such as for example 10 to 30 neoantigens.

In an embodiment, the peptide antigens are not pre-selected based on any characteristic relating to isoelectric point, solubility, stability and/or immunogenicity. In an embodiment, the peptide antigens in the composition have one or more different characteristics relating to isoelectric point, solubility, stability and/or immunogenicity. For example, the peptide antigens may have a different length, sequence, molecular weight, charge, polarity, hydrophobicity and/or hydrophilicity.

In an embodiment of the compositions disclosed herein, each of the peptide antigens is, independently, at a concentration of between about 0.05 μg/μl and about 10 μg/μl, 0.1 μg/μl and about 5.0 μg/μl, or about 0.5 μg/μl and about 1.0 μg/μl. In an embodiment of the compositions disclosed herein, each of the peptide antigens is, independently, at a concentration of about 0.1 μg/μl, 0.25 μg/μl, about 0.5 μg/μl, about 0.75 μg/μl, about 1.0 μg/μl, about 1.25 μg/μl, about 1.5 μg/μl, about 1.75 μg/μl, about 2.0 μg/μl, about 2.25 μg/μl or about 2.5 μg/μl. By “independently” it is meant that the amount of each peptide antigen in the composition is independent of the amount of any other and, therefore, each respective peptide antigen may have the same or different concentration as any other peptide antigen. In an embodiment, each of the peptide antigens in the composition is at a concentration of about 0.5 μg/μl.

In an embodiment, the pharmaceutical composition comprises 10 or more different peptide antigens and each peptide antigen is at a concentration of at least about 0.5 μg/μl. In an embodiment, each of these 10 or more peptide antigens are, independently, 20-30 amino acids in length.

The pharmaceutical compositions disclosed herein comprise a hydrophobic carrier. As used herein, a “hydrophobic carrier” refers to a liquid hydrophobic substance. The term “hydrophobic carrier” may be referred to herein interchangeably as an “oil-based carrier”.

The hydrophobic carrier may be an essentially pure hydrophobic substance or a mixture of hydrophobic substances. Hydrophobic substances that are useful in the methods and compositions described herein are those that are pharmaceutically and/or immunologically acceptable. The carrier is typically a liquid at room temperature (e.g. about 18-25° C.), but certain hydrophobic substances that are not liquids at room temperature may be liquefied, for example by warming, and may also be useful.

Oil or a mixture of oils is a particularly suitable carrier for use in the methods and compositions disclosed herein. Oils should be pharmaceutically and/or immunologically acceptable. Suitable oils include, for example, mineral oils (especially light or low viscosity mineral oil such as Drakeol® 6VR), vegetable oils (e.g., soybean oil such as MS80), nut oils (e.g., peanut oil), or mixtures thereof. Thus, in an embodiment the hydrophobic carrier is a hydrophobic substance such as vegetable oil, nut oil or mineral oil. Animal fats and artificial hydrophobic polymeric materials, particularly those that are liquid at atmospheric temperature or that can be liquefied relatively easily, may also be used.

In some embodiments, the hydrophobic carrier may be, or comprise, Incomplete Freund's Adjuvant (IFA), a mineral oil-based model hydrophobic carrier. In another embodiment, the hydrophobic carrier may be, or comprise, a mannide oleate in mineral oil solution, such as that commercially available as Montanide® ISA 51 (SEPPIC, France). While these carriers are commonly used to prepare water-in-oil emulsions, the present disclosure relates to water-free compositions. As such, these carriers are not emulsified with water in the methods and compositions disclosed herein.

In an embodiment, the hydrophobic carrier is mineral oil or a mannide oleate in mineral oil solution.

In an embodiment, the hydrophobic carrier is Montanide® ISA 51.

In an embodiment, the hydrophobic carrier is MS80 oil, which is a mixture of mineral oil (Sigma Aldrich) and Span80 (Fluka). The components can be purchased separately and mixed prior to use.

The compositions disclosed herein may further comprise one or more additional components as are known in the art (see e.g. Remington's Pharmaceutical Sciences, Mack Publishing Company, Easton, Pa., USA 1985; and The United States Pharmacopoeia: The National Formulary (USP 24 NF19) published in 1999).

In an embodiment, the compositions may additionally comprise an adjuvant, a T-helper epitope, a surfactant and/or an excipient. Exemplary and non-limiting embodiments of adjuvants, T-helper epitopes and surfactants that may be used are described below. In an embodiment, the composition comprises a T-helper epitope and/or adjuvant if the therapeutic agent is one or more peptide antigens.

In an embodiment, the pharmaceutical composition is a clear solution. In an embodiment, the pharmaceutical composition has no visible precipitate.

The ability to prepare a composition as disclosed herein may have significant advantages, particularly in the field of personalized cancer medicines involving neoantigens.

In this regard, in an embodiment, the present disclosure relates to a stable, water-free pharmaceutical composition comprising one or more lipid-based structures having a single lipid layer assembly, five or more different peptide neoantigens, and a hydrophobic carrier, wherein the peptide neoantigens are not pre-selected based on any characteristic relating to isoelectric point, solubility, stability and/or immunogenicity. In this embodiment, the terms “stable” and “water-free” have the same meanings as described herein. Also, the lipid-based structures having a single lipid layer assembly and hydrophobic carrier are as described herein.

In an embodiment of the neoantigen composition, the neoantigens have a different length, sequence, molecular weight, charge, polarity, hydrophobicity and/or hydrophilicity.

As discussed herein, pharmaceutical compositions comprising neoantigens involve special considerations in that they typically have to be formulated within weeks to facilitate an effective immune response upon immunization. Timing is critical and poses significant hurdles. Unlike other types of pharmaceutical formulations, there is often little or no time to perform steps to select appropriate peptide antigens based on isoelectric point, solubility, stability and/or immunogenicity. Without these selectivity steps, it is an obstacle to formulate a stable and therapeutically effective pharmaceutical composition with more than even a single peptide antigen.

However, as shown in Example 4, multiple peptide neoantigens were capable of being solubilized in a single composition without any pre-selection based on characteristics relating to isoelectric point, solubility, stability and/or immunogenicity. Thus, it is believed that the compositions will alleviate the requirements to perform time-consuming selection steps to identify the so-called “best” antigen for personalized vaccines. Rather, the compositions disclosed herein permit the inclusion of multiple different peptide neoantigens (e.g. five or more) each at an effective amount to induce an immune response, without the need for antigen selection steps. Indeed, as shown herein, even with a large peptide antigen payload, the neoantigen compositions retain the ability to promote rapid and enhanced immune responses after a single administration (Example 6; FIG. 17).

Immune Responses and Treatment Indications

The compositions disclosed herein may find application in any instance in which it is desired to administer a therapeutic agent to a subject. The subject may be a vertebrate, such as a fish, bird or mammal. In an embodiment, the subject is a mammal. In an embodiment, the subject is a human.

In an embodiment, the compositions may be used in methods for treating, preventing or diagnosing a disease, disorder or condition to which the therapeutic agent is targeted. In an embodiment, the methods comprise administering to a subject the pharmaceutical composition as described herein.

In an embodiment, the compositions may be used in methods for modulating an immune response in a subject. As used herein, the term “modulating” is intended to refer to both immunostimulation (e.g. inducing or enhancing an immune response) and immunosuppression (e.g. preventing or decreasing an immune response). Typically, the method would involve one or the other of immunostimulation or immunosuppression, but it is possible that the method could be directed to both. As referred to herein, the “immune response” may either be a cell-mediated (CTL) immune response or an antibody (humoral) immune response.

In some embodiments, the compositions disclosed herein may be used for inducing a cell-mediated immune response to the therapeutic agents (e.g. peptide antigens).

As used herein, to “induce” an immune response is to elicit and/or potentiate an immune response. Inducing an immune response encompasses instances where the immune response is initiated, enhanced, elevated, improved or strengthened to the benefit of the host relative to the prior immune response status, for example, before the administration of a composition disclosed herein.

As used herein, the terms “cell-mediated immune response”, “cellular immunity”, “cellular immune response” or “cytotoxic T-lymphocyte (CTL) immune response” (used interchangeably herein) refer to an immune response characterized by the activation of macrophages and natural killer cells, the production of antigen-specific cytotoxic T lymphocytes and/or the release of various cytokines in response to an antigen. Cytotoxic T lymphocytes are a sub-group of T lymphocytes (a type of white blood cell) which are capable of inducing the death of infected somatic or tumor cells; they kill cells that are infected with viruses (or other pathogens), or that are otherwise damaged or dysfunctional.

Most cytotoxic T cells express T cell receptors that can recognise a specific peptide antigen bound to Class I MEW molecules. Typically, cytotoxic T cells also express CD8 (i.e. CD8+ T cells), which is attracted to portions of the Class I MEW molecule. This affinity keeps the cytotoxic T cell and the target cell bound closely together during antigen-specific activation.

Cellular immunity protects the body by, for example, activating antigen-specific cytotoxic T-lymphocytes (e.g. antigen-specific CD8+ T cells) that are able to lyse body cells displaying epitopes of foreign or mutated antigen on their surface, such as cancer cells displaying tumor-specific antigens (e.g. neoantigens); activating macrophages and natural killer cells, enabling them to destroy intracellular pathogens; and stimulating cells to secrete a variety of cytokines that influence the function of other cells involved in adaptive immune responses and innate immune responses.

Cellular immunity is an important component of the adaptive immune response and following recognition of antigen by cells through their interaction with antigen-presenting cells such as dendritic cells, B lymphocytes and to a lesser extent, macrophages, protect the body by various mechanisms such as:

1. activating antigen-specific cytotoxic T-lymphocytes that are able to induce apoptosis in body cells displaying epitopes of foreign or mutated antigen on their surface, such as cancer cells displaying tumor-specific antigens;

2. activating macrophages and natural killer cells, enabling them to destroy intracellular pathogens; and

3. stimulating cells to secrete a variety of cytokines that influence the function of other cells involved in adaptive immune responses and innate immune responses.

Cell-mediated immunity is most effective in removing virus-infected cells, but also participates in defending against fungi, protozoans, cancers, and intracellular bacteria. It also plays a major role in transplant rejection.

Since cell-mediated immunity involves the participation of various cell types and is mediated by different mechanisms, several methods could be used to demonstrate the induction of immunity following vaccination. These could be broadly classified into detection of: i) specific antigen presenting cells; ii) specific effector cells and their functions and iii) release of soluble mediators such as cytokines.

i) Antigen presenting cells: Dendritic cells and B cells (and to a lesser extent macrophages) are equipped with special immunostimulatory receptors that allow for enhanced activation of T cells, and are termed professional antigen presenting cells (APC). These immunostimulatory molecules (also called co-stimulatory molecules) are up-regulated on these cells following infection or vaccination, during the process of antigen presentation to effector cells such as CD4 and CD8 cytotoxic T cells. Such co-stimulatory molecules (such as CD40, CD80, CD86, MHC class I or MHC class II) can be detected, for example, by using flow cytometry with fluorochrome-conjugated antibodies directed against these molecules along with antibodies that specifically identify APC (such as CD11c for dendritic cells).

ii) Cytotoxic T cells: (also known as Tc, killer T cell, or cytotoxic T-lymphocyte (CTL)) are a sub-group of T cells which induce the death of cells that are infected with viruses (and other pathogens), or expressing tumor antigens. These CTLs directly attack other cells carrying certain foreign or abnormal molecules on their surface. The ability of such cellular cytotoxicity can be detected using in vitro cytolytic assays (chromium release assay). Thus, induction of adaptive cellular immunity can be demonstrated by the presence of such cytotoxic T cells, wherein, when antigen loaded target cells are lysed by specific CTLs that are generated in vivo following vaccination or infection.

Naive cytotoxic T cells are activated when their T cell receptor (TCR) strongly interacts with a peptide-bound MHC class I molecule. This affinity depends on the type and orientation of the antigen/MHC complex, and is what keeps the CTL and infected cell bound together. Once activated the CTL undergoes a process called clonal expansion in which it gains functionality, and divides rapidly, to produce an army of “armed”-effector cells. Activated CTL will then travel throughout the body in search of cells bearing that unique MHC Class I+peptide. This could be used to identify such CTLs in vitro by using peptide-MHC Class I tetramers in flow cytometric assays.

When exposed to these infected or dysfunctional somatic cells, effector CTL release perforin and granulysin: cytotoxins which form pores in the target cell's plasma membrane, allowing ions and water to flow into the infected cell, and causing it to burst or lyse. CTL release granzyme, a serine protease that enters cells via pores to induce apoptosis (cell death). Release of these molecules from CTL can be used as a measure of successful induction of cell-mediated immune response following vaccination. This can be done by enzyme linked immunosorbant assay (ELISA) or enzyme linked immunospot assay (ELISPOT) where CTLs can be quantitatively measured. Since CTLs are also capable of producing important cytokines such as IFN-γ, quantitative measurement of IFN-γ-producing CD8 cells can be achieved by ELISPOT and by flowcytometric measurement of intracellular IFN-γ in these cells.

CD4+“helper” T cells: CD4+ lymphocytes, or helper T cells, are immune response mediators, and play an important role in establishing and maximizing the capabilities of the adaptive immune response. These cells have no cytotoxic or phagocytic activity; and cannot kill infected cells or clear pathogens, but, in essence “manage” the immune response, by directing other cells to perform these tasks. Two types of effector CD4+T helper cell responses can be induced by a professional APC, designated Th1 and Th2, each designed to eliminate different types of pathogens.

Helper T cells express T cell receptors (TCR) that recognize antigen bound to Class II MHC molecules. The activation of a naive helper T cell causes it to release cytokines, which influences the activity of many cell types, including the APC that activated it. Helper T cells require a much milder activation stimulus than cytotoxic T cells. Helper T cells can provide extra signals that “help” activate cytotoxic cells. Two types of effector CD4+T helper cell responses can be induced by a professional APC, designated Th1 and Th2, each designed to eliminate different types of pathogens. The two Th cell populations differ in the pattern of the effector proteins (cytokines) produced. In general, Th1 cells assist the cell-mediated immune response by activation of macrophages and cytotoxic T cells; whereas Th2 cells promote the humoral immune response by stimulation of B cells for conversion into plasma cells and by formation of antibodies. For example, a response regulated by Th1 cells may induce IgG2a and IgG2b in mouse (IgG1 and IgG3 in humans) and favor a cell mediated immune response to an antigen. If the IgG response to an antigen is regulated by Th2 type cells, it may predominantly enhance the production of IgG1 in mouse (IgG2 in humans). The measure of cytokines associated with Th1 or Th2 responses will give a measure of successful vaccination. This can be achieved by specific ELISA designed for Th1-cytokines such as IFN-γ, IL-2, IL-12, TNF-α and others, or Th2-cytokines such as IL-4, IL-5, IL10 among others.

iii) Measurement of cytokines: released from regional lymph nodes gives a good indication of successful immunization. As a result of antigen presentation and maturation of APC and immune effector cells such as CD4 and CD8 T cells, several cytokines are released by lymph node cells. By culturing these LNC in vitro in the presence of antigen, an antigen-specific immune response can be detected by measuring release if certain important cytokines such as IFN-γ, IL-2, IL-12, TNF-α and GM-CSF. This could be done by ELISA using culture supernatants and recombinant cytokines as standards.

Successful immunization may be determined in a number of ways known to the skilled person including, but not limited to, hemagglutination inhibition (HAIJ) and serum neutralization inhibition assays to detect functional antibodies; challenge studies, in which vaccinated subjects are challenged with the associated pathogen to determine the efficacy of the vaccination; and the use of fluorescence activated cell sorting (FACS) to determine the population of cells that express a specific cell surface marker, e.g. in the identification of activated or memory lymphocytes. A skilled person may also determine if immunization with a composition as disclosed herein elicited an antibody and/or cell mediated immune response using other known methods. See, for example, Coligan et al., ed. Current Protocols in Immunology, Wiley Interscience, 2007.

In an embodiment, the composition disclosed herein is capable of generating an enhanced cell-mediated immune response against one or more of the therapeutic agents (e.g. peptide antigens) in the composition that is at least 2-fold, at least 3-fold, at least 4-fold, at least 5-fold, at least 6-fold, at least 7-fold, at least 8-fold, at least 9-fold or at least 10-fold greater than when the antigens are formulated in an aqueous-based vaccine formulation. By “aqueous-based vaccine”, it is meant a vaccine that comprises identical components as the compositions disclosed herein, with the exception that the hydrophobic carrier is replaced with an aqueous carrier and the aqueous-based vaccine does not comprise lipid-based structures.

In an embodiment, the composition disclosed herein is capable of generating the enhanced cell-mediated immune response with only a single administration of the composition. Thus, in an embodiment, the compositions disclosed herein are for delivery of the therapeutic agents (e.g. peptide antigens) by single administration.

In an embodiment, the compositions disclosed herein may be used for inducing an antibody immune response to the therapeutic agents (e.g. peptide antigens).

An “antibody immune response” or “humoral immune response” (used interchangeably herein), as opposed to cell-mediated immunity, is mediated by secreted antibodies which are produced in the cells of the B lymphocyte lineage (B cells). Such secreted antibodies bind to antigens, such as for example those on the surfaces of foreign substances, pathogens (e.g. viruses, bacteria, etc.) and/or cancer cells, and flag them for destruction.

As used herein, “humoral immune response” refers to antibody production and may also include, in addition or alternatively, the accessory processes that accompany it, such as for example the generation and/or activation of T-helper 2 (Th2) or T-helper 17 (Th17) cells, cytokine production, isotype switching, affinity maturation and memory cell activation. “Humoral immune response” may also include the effector functions of an antibody, such as for example toxin neutralization, classical complement activation, and promotion of phagocytosis and pathogen elimination. The humoral immune response is often aided by CD4+Th2 cells and therefore the activation or generation of this cell type may also be indicative of a humoral immune response.

An “antibody” is a protein comprising one or more polypeptides substantially or partially encoded by immunoglobulin genes or fragments of immunoglobulin genes. The recognized immunoglobulin genes include the κ, λ, α, γ, δ, ε and μconstant region genes, as well as myriad immunoglobulin variable region genes. Light chains are classified as either κ or λ. Heavy chains are classified as γ, α, δ, or ε, which in turn define the immunoglobulin classes, IgG, IgM, IgA, IgD and IgE, respectively. A typical immunoglobulin (antibody) structural unit comprises a protein containing four polypeptides. Each antibody structural unit is composed of two identical pairs of polypeptide chains, each having one “light” and one “heavy” chain. The N-terminus of each chain defines a variable region primarily responsible for antigen recognition. Antibody structural units (e.g. of the IgA and IgM classes) may also assemble into oligomeric forms with each other and additional polypeptide chains, for example as IgM pentamers in association with the J-chain polypeptide.

Antibodies are the antigen-specific glycoprotein products of a subset of white blood cells called B lymphocytes (B cells). Engagement of antigen with antibody expressed on the surface of B cells can induce an antibody response comprising stimulation of B cells to become activated, to undergo mitosis and to terminally differentiate into plasma cells, which are specialized for synthesis and secretion of antigen-specific antibody.

B cells are the sole producers of antibodies during an immune response and are thus a key element to effective humoral immunity. In addition to producing large amounts of antibodies, B cells also act as antigen-presenting cells and can present antigenic peptide to T cells, such as T helper CD4 or cytotoxic CD8+ T cells, thus propagating the immune response. B cells, as well as T cells, are part of the adaptive immune response. During an active immune response, induced for example by either vaccination or natural infection, antigen-specific B cells are activated and clonally expand. During expansion, B cells evolve to have higher affinity for the epitope. Proliferation of B cells can be induced indirectly by activated T-helper cells, and also directly through stimulation of receptors, such as the TLRs.

Antigen presenting cells, such as dendritic cells and B cells, are drawn to vaccination sites and can interact with antigens and adjuvants contained in a vaccine composition. Typically, the adjuvant stimulates the cells to become activated and the antigen provides the blueprint for the target. Different types of adjuvants may provide different stimulation signals to cells. For example, polyI:C (a TLR3 agonist) can activate dendritic cells, but not B cells. Adjuvants such as Pam3Cys, Pam2Cys and FSL-1 are especially adept at activating and initiating proliferation of B cells, which is expected to facilitate the production of an antibody response (Moyle 2008; So 2012).

A humoral immune response is one of the common mechanisms for effective infectious disease vaccines (e.g. to protect against viral or bacterial invaders). However, a humoral immune response can also be useful for combating cancer. Whereas a cancer vaccine is typically designed to produce a cell-mediated immune response that can recognize and destroy cancer cells, B cell mediated responses may target cancer cells through other mechanisms which may in some instances cooperate with a cytotoxic T cell for maximum benefit. Examples of B cell mediated (e.g. humoral immune response mediated) anti-tumor responses include, without limitation: 1) Antibodies produced by B cells that bind to surface antigens (e.g. neoantigens) found on tumor cells or other cells that influence tumorigenesis. Such antibodies can, for example. induce killing of target cells through antibody-dependant cell-mediated cytotoxicity (ADCC) or complement fixation, potentially resulting in the release of additional antigens that can be recognized by the immune system; 2) Antibodies that bind to receptors on tumor cells to block their stimulation and in effect neutralize their effects; 3) Antibodies that bind to factors released by or associated with a tumor or tumor-associated cells to modulate a signaling or cellular pathway that supports cancer; and 4) Antibodies that bind to intracellular targets and mediate anti-tumor activity through a currently unknown mechanism.

One method of evaluating an antibody response is to measure the titers of antibodies reactive with a particular antigen. This may be performed using a variety of methods known in the art such as enzyme-linked immunosorbent assay (ELISA) of antibody-containing substances obtained from animals. For example, the titers of serum antibodies which bind to a particular antigen may be determined in a subject both before and after exposure to the antigen. A statistically significant increase in the titer of antigen-specific antibodies following exposure to the antigen would indicate the subject had mounted an antibody response to the antigen.

Without limitation, other assays that may be used to detect the presence of an antigen-specific antibody include immunological assays (e.g. radioimmunoassay (MA)), immunoprecipitation assays, and protein blot (e.g. Western blot) assays; and neutralization assays (e.g., neutralization of viral infectivity in an in vitro or in vivo assay).

The compositions disclosed herein may be useful for treating or preventing diseases and/or disorders ameliorated by a cell-mediated immune response or a humoral immune response. The compositions disclosed herein may find application in any instance in which it is desired to administer therapeutic agents (e.g. peptide antigens) to a subject to induce a cell-mediated immune response or a humoral immune response. In an embodiment, the compositions may find application for the delivery of a personalized vaccine, e.g. comprising neoantigens.

In an embodiment, the present disclosure relates to a method comprising administering the composition as described herein to a subject in need thereof. In an embodiment, the method is for the treatment and/or prevention of a disease, disorder or condition in a subject. In an embodiment, the method is for the treatment and/or prevention of an infectious disease or cancer.

In an embodiment, the method is for inducing an antibody immune response and/or cell-mediated immune response to the therapeutic agents (e.g. peptide antigens) in said subject. In an embodiment, such method is for the treatment and/or prevention of an infectious disease or cancer.

“Treating” or “treatment of”, or “preventing” or “prevention of”, as used herein, refers to an approach for obtaining beneficial or desired results. Beneficial or desired results can include, but are not limited to, alleviation or amelioration of one or more symptoms or conditions, diminishment of extent of disease, stabilisation of the state of disease, prevention of development of disease, prevention of spread of disease, delay or slowing of disease progression (e.g. suppression), delay or slowing of disease onset, conferring protective immunity against a disease-causing agent and amelioration or palliation of the disease state. “Treating” or “preventing” can also mean prolonging survival of a patient beyond that expected in the absence of treatment and can also mean inhibiting the progression of disease temporarily or preventing the occurrence of disease, such as by preventing infection in a subject. “Treating” or “preventing” may also refer to a reduction in the size of a tumor mass, reduction in tumor aggressiveness, etc.

Treating” may be distinguished from “preventing” in that “treating” typically occurs in a subject who already has a disease or disorder, or is known to have already been exposed to an infectious agent, whereas “preventing” typically occurs in a subject who does not have a disease or disorder, or is not known to have been exposed to an infectious agent. As will be appreciated, there may be overlap in treatment and prevention. For example, it is possible to be “treating” a disease in a subject, while at same time “preventing” symptoms or progression of the disease. Moreover, at least in the context of vaccination, “treating” and “preventing” may overlap in that the treatment of a subject is to induce an immune response that may have the subsequent effect of preventing infection by a pathogen or preventing the underlying disease or symptoms caused by infection with the pathogen. These preventive aspects are encompassed herein by expressions such as “treatment of an infectious disease” or “treatment of cancer”.

In an embodiment, the compositions disclosed herein may be used for treating and/or preventing an infectious disease, such as caused by a viral infection, in a subject in need thereof. The subject may be infected with a virus or may be at risk of developing a viral infection. Viral infections that may be treated and/or prevented by the use or administration of a composition as disclosed herein, without limitation, Cowpoxvirus, Vaccinia virus, Pseudocowpox virus, Human herpesvirus 1, Human herpesvirus 2, Cytomegalovirus, Human adenovirus A-F, Polyomavirus, Human papillomavirus (HPV), Parvovirus, Hepatitis A virus, Hepatitis B virus, Hepatitis C virus, Human immunodeficiency virus, Orthoreovirus, Rotavirus, Ebola virus, parainfluenza virus, influenza A virus, influenza B virus, influenza C virus, Measles virus, Mumps virus, Rubella virus, Pneumovirus, respiratory syncytial virus (RSV), Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus and varicella. In a particular embodiment, the viral infection is Human papillomavirus, Ebola virus, respiratory syncytial virus or an influenza virus.

In an embodiment, the compositions disclosed herein may be used for treating and/or preventing an infectious disease, such as caused by a non-viral pathogen (such as a bacterium or protozoan) in a subject in need thereof. The subject may be infected with the pathogen or may be at risk of developing an infection by the pathogen. Without limitation, exemplary bacterial pathogens may include Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli O157: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica. In a particular embodiment, the bacterial infection is Anthrax. Without limitation, exemplary protozoan pathogens may include those of the genus Plasmodium (Plasmodium falciparum, Plasmodium malariae, Plasmodium vivax, Plasmodium ovale or Plasmodium knowlesi), which cause malaria.

In an embodiment, the compositions disclosed herein may be for use in treating and/or preventing cancer in a subject in need thereof. The subject may have cancer or may be at risk of developing cancer.

As used herein, the terms “cancer”, “cancer cells”, “tumor” and “tumor cells”, (used interchangeably) refer to cells that exhibit abnormal growth, characterized by a significant loss of control of cell proliferation or cells that have been immortalized. The term “cancer” or “tumor” includes metastatic as well as non-metastatic cancer or tumors. A cancer may be diagnosed using criteria generally accepted in the art, including the presence of a malignant tumor.

Without limitation, cancers that may be capable of being treated and/or prevented by the use or administration of a composition as disclosed herein include carcinoma, adenocarcinoma, lymphoma, leukemia, sarcoma, blastoma, myeloma, and germ cell tumors. Without limitation, particularly suitable embodiments may include glioblastoma, multiple myeloma, ovarian cancer, breast cancer, fallopian tube cancer, prostate cancer or peritoneal cancer. In one embodiment, the cancer may be caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukaemia virus-1. In an embodiment, the cancer is one that expresses one or more tumor-specific neoantigens.

In a particular embodiment, the cancer is breast cancer, ovarian cancer, prostate cancer, fallopian tube cancer, peritoneal cancer, glioblastoma or diffuse large B cell lymphoma.

The methods and compositions disclosed herein may be useful for either the treatment or prophylaxis of cancer; for example, a reduction of the severity of cancer (e.g. size of the tumor, aggressiveness and/or invasiveness, malignancy, etc.) or the prevention of cancer recurrences.

In an embodiment, the method for treating and/or preventing cancer first comprises identifying one or more neoantigens or neoepitopes in the patients' tumor cells. The skilled person will understand methods known in the art that can be used to identify the one or more neoantigens (see, for example, Srivastava 2015 and the references cited therein). As an exemplary embodiment, whole genome/exome sequencing may be used to identify mutated neoantigens that are uniquely present in a tumor of an individual patient. The collection of identified neoantigens can be analyzed to select (e.g. based on algorithms) a specific, optimized subset of neoantigens and/or neoepitopes for use as a personalized cancer vaccine.

Having identified and selected one or more neoantigens, one of skill in the art will appreciate that there are a variety of ways in which to produce such neoantigens either in vitro or in vivo. The neoantigenic peptides may be produced by any method known the art and then may be formulated into a composition or kit as described herein and administered to a subject.

In an embodiment, upon administration to a subject, the composition induces a tumor-specific immune response in the treatment of cancer. By this it is meant that the immune response specifically targets the tumor cells without a significant effect on normal cells of the body which do not express the neoantigen. Further, in an embodiment, the composition may comprise at least one patient-specific neoepitope such that the tumor-specific immune response is patient-specific for the subject or a subset of subjects, i.e. a personalized immunotherapy.

The composition as disclosed herein may be administered by any suitable route. In an embodiment, the route of administration is subcutaneous injection.

In an embodiment in which the composition is for administration by injection, the pharmaceutical compositions as disclosed herein may be formulated as a microdose. As used herein, by “microdose volume” it is meant a single dose volume of less than 100 μl. In some embodiments, the microdose volume is about 50 about 55 about 60 μl, about 65 μl, about 70 μl, about 75 μl, about 80 about 85 about 90 μl or about 95 μl of the composition. In some embodiments, the microdose volume is between about 50 μl to about 75 μl of the composition. In some embodiments, the microdose volume is about 50 μl or exactly 50 μl. In an embodiment, by practice of the methods disclosed herein and use of the compositions disclosed herein, the microdose volume is capable of being formulated with multiple different peptide antigens at a total peptide antigen concentration of more than 5 μg in the microdose, and the microdose volume is capable of inducing an antibody and/or CTL immune response in a human subject.

Kits

The compositions disclosed herein are optionally provided to a user as a kit. In an embodiment, the kit is for preparing a composition for the treatment, prevention and/or diagnosis of a disease, disorder or condition. In an embodiment, the kit is for preparing a composition for inducing an antibody and/or CTL immune response.

In an embodiment, a kit of the present disclosure comprises a container comprising a dried lipid/therapeutic agent preparation prepared by the methods disclosed herein and a container comprising a hydrophobic carrier. The dried lipid/therapeutic agent preparation may be any of those described herein. In an embodiment, the dried lipid/therapeutic agent preparation comprises ten or more different peptide antigens. In an embodiment, the peptide antigens are neoantigens. The hydrophobic carrier is as described herein, and in an embodiment is mineral oil or a mannide oleate in mineral oil solution.

In another embodiment, a kit of the present disclosure comprises a container comprising a dried lipid/therapeutic agent preparation prepared by the methods disclosed herein. In such embodiment, the kit does not include the hydrophobic carrier, but rather the hydrophobic carrier is supplied separately or is already in possession by the end user. In an embodiment, the dried lipid/therapeutic agent preparation comprises ten or more different peptide antigens. In an embodiment, the peptide antigens are neoantigens.

The kits can further comprise one or more additional reagents, packaging materials, and an instruction set or user manual detailing preferred methods of using the kit components. In an embodiment, the containers are vials.

Components of the Methods, Dried Preparations, Compositions, Uses & Kits

The methods, dried preparations, compositions, uses and kits disclosed herein are used with or comprise one or more therapeutic agents and may further be used with or comprise, without limitation, one or more additional components, such as for example a T-helper epitope, an adjuvant and a surfactant. While exemplary embodiments of these components are described herein, it will be appreciated that other components may also be used, such as excipients, preservatives, or other inactive ingredients.

As used herein, the term “therapeutic agent” does not include or encompass a T-helper epitope or an adjuvant, which are separately described below and are different components that may or may not be included in the methods, dried preparations, compositions, uses and kits disclosed herein. Further, in an embodiment, a T-helper epitope and/or an adjuvant are included only when the therapeutic agent is an antigen.

Therapeutic Agents

Therapeutic agents that can be used in the methods, dried preparations, compositions, uses and kits disclosed herein include any molecule, substance or compound that is capable of providing a therapeutic activity, response or effect in the treatment or prevention of a disease, disorder or condition, including diagnostic and prophylactic agents. The term “therapeutic agent” includes molecules, compounds and substances, or parts thereof, commonly referred to as “active pharmaceutical ingredients” or “active ingredients”, which represent the component of a medicine that is biologically active.

As used herein, the “therapeutic agent” is not a T-helper epitope or an adjuvant, which are described separately below.

The therapeutic agents include antigens, drugs and other agents including, but not limited to, those listed in the United States Pharmacopeia and in other known pharmacopeias. Therapeutic agents may be used in the practice of the present invention with or without any chemical modification. Therapeutic agents include proteins, polypeptides, peptides, polynucleotides, polysaccharides, and drugs (e.g. small molecules or biologics).

In an embodiment, the therapeutic agent is a peptide antigen, a DNA or RNA polynucleotide that encodes a polypeptide, a hormone, a cytokine, an allergen, a catalytic DNA (deoxyribozyme), a catalytic RNA (ribozyme), an antisense RNA, an interfering RNA, an antagomir, a small molecule drug, a biologic drug, an antibody, or a fragment or derivative of any one thereof; or a mixture thereof.

In an embodiment, the methods disclosed herein are for formulating a mixture of multiple different therapeutic agents in a single composition. In an embodiment, the methods disclosed herein are for formulating a mixture of 2, 3, 4, 5, 6, 7, 8, 9, 10, 11, 12, 13, 14, 15, 16, 17, 18, 19, 20, 21, 22, 23, 24, 25, 26, 27, 28, 29, 30 or more different therapeutic agents in a single composition. In an embodiment, the methods disclosed herein are for formulating a mixture of 10-20 different therapeutic agents in a single composition.

In embodiments involving multiple different therapeutic agents, in one aspect all of the therapeutic agents may be of the same type (e.g. all peptide antigens, all small molecule drugs, all polynucleotides encoding polypeptides, etc.). In other aspects, the therapeutic agents may be of different types (e.g. one or more peptide antigens in combination with one or more small molecule drugs).

In an embodiment, the therapeutic agent is one that is not compatible (e.g. insoluble or unstable) with one or both of an aqueous solution or a hydrophobic solution or both. In an embodiment, the therapeutic agent is hydrophilic or substantially hydrophilic and is not naturally compatible in a hydrophobic environment. In an embodiment, the therapeutic agent is hydrophobic or substantially hydrophobic and is not naturally compatible in a hydrophilic (e.g. aqueous) environment. In an embodiment, the therapeutic agent is one that is not compatible with size extrusion procedures (e.g. precipitates under high pressure extrusion through a membrane, such as 5000 psi back-pressure and a 0.22 μm membrane).

Exemplary embodiments of therapeutics agents are described below, without limitation.

Peptide Antigens

In an embodiment, the therapeutic agent is one or more peptide antigens.

In an embodiment, the methods disclosed herein may be used to prepare a pharmaceutical composition comprising a single peptide antigen. In an embodiment, the methods disclosed herein may be used to prepare a pharmaceutical composition comprising up to 30 or more different solubilized peptide antigens.

In an embodiment, the methods disclosed herein may be used to prepare a pharmaceutical composition comprising two or more different peptide antigens. In an embodiment, the methods disclosed herein may be used to prepare a pharmaceutical composition comprising 2, 3, 4, 5, 6, 7, 8, 9, 10, 11, 12, 13, 14, 15, 16, 17, 18, 19, 20, 21, 22, 23, 24, 25, 26, 27, 28, 29, 30 or more different peptide antigens. In an embodiment, the methods disclosed herein may be used to prepare a pharmaceutical composition comprising 2 to 30 different solubilized peptide antigens, 5 to 30 different peptide antigens, 10 to 20 different peptide antigens, or 10 to 15 different peptide antigens. In an embodiment, the methods disclosed herein may be used to prepare a pharmaceutical composition comprising five or more different solubilized peptide antigens. In an embodiment, the methods disclosed herein may be used to prepare a pharmaceutical composition comprising 10, 11, 12, 13, 14, 15, 16, 17, 18, 19 or 20 different peptide antigens.

As used herein, the term “antigen” refers to any substance or molecule that can bind specifically to components of the immune system. In some embodiments, suitable antigens are those that are capable of inducing or generating an immune response in a subject. An antigen that is capable of inducing an immune response is said to be immunogenic, and may also be called an immunogen. Thus, as used herein, the term “antigen” includes immunogens and the terms may be used interchangeably unless specifically stated otherwise.

As used herein, the term “peptide antigen” is an antigen as defined above that is a protein or a polypeptide. In an embodiment, the peptide antigen may be derived from a microorganism, such as for example a live, attenuated, inactivated or killed bacterium, virus or protozoan, or part thereof. In an embodiment, the peptide antigen may be derived from an animal, such as for example a human, or an antigen that is substantially related thereto.

As used herein, the term “derived from” encompasses, without limitation: a peptide antigen that is isolated or obtained directly from an originating source (e.g. a subject); a synthetic or recombinantly generated peptide antigen that is identical or substantially related to a peptide antigen from an originating source; or a peptide antigen which is made from a peptide antigen of an originating source or a fragment thereof. When it is stated that a peptide antigen is “from” a source, the term “from” may be equated with “derived from”. The term “substantially related”, in this context, means that the peptide antigen may have been modified by chemical, physical or other means (e.g. sequence modification), but that the resultant product remains capable of generating an immune response to the original peptide antigen and/or to the disease or disorder associated with the original antigen. “Substantially related” includes variants and/or derivatives of the native peptide antigen.

In an embodiment, the peptide antigen can be isolated from a natural source. In some embodiments, the peptide antigen may be purified to be from about 90% to about 95% pure, from about 95% to about 98% pure, from about 98% to about 99% pure, or greater than 99% pure.

In an embodiment, the peptide antigen can be recombinantly generated, such as for example by expression in vitro or in vivo.

In an embodiment, the peptide antigen is a synthetically produced polypeptide based on a sequence of amino acids of a native target protein. The peptide antigen can be synthesized, in whole or in part, using chemical methods well known in the art (see e.g., Caruthers 1980, Horn 1980, Banga 1995). For example, peptide synthesis can be performed using various solid-phase techniques (see e.g., Roberge 1995, Merrifield 1997) and automated synthesis may be achieved, e.g., using the ABI 431A Peptide Synthesizer (Perkin Elmer) in accordance with the instructions provided by the manufacturer.

As used interchangeably herein, the terms “variant” or “modified variant” refer to therapeutic agents that have been modified by any chemical, physical or other means to provide an altered therapeutic agent. The modified variant may have one or more improved characteristics as compared to the unmodified counterpart (e.g. solubility, stability, activity, etc.). Depending on the type of therapeutic agent (e.g. peptide antigen, hormone, catalytic DNA or RNA, etc.), different types of modifications may be known in the art and may be applied to prepare a modified variant.

In the context of peptide antigens, many different types of peptide modifications are known in the art and may be used in the practice of the present invention. For example, and without limitation, the peptide antigen may be modified to improve its solubility, stability and/or immunogenicity. Non-limiting examples of modifications that may be made include N-terminal modifications, C-terminal modifications, amidation, acetylation, peptide cyclization by creating disulfide bridges, phosphorylation, methylation, conjugation to other molecules (e.g. BSA, KLH, OVA), PEGylation and the inclusion of unnatural amino acids.

In an embodiment, the modification may be an amino acid sequence modification, e.g. deletion, substitution or insertion. The substitution may be a conservative amino acid substitution or a non-conservative amino acid substitution. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing. Specific, non-limiting examples of a conservative substitution include the following examples:

| Original Residue | Conservative Substitution |
|---|---|
| Ala | Ser |
| Arg | Lys |
| Asn | Gln, His |
| Asp | Glu |
| Cys | Ser |
| Gln | Asn |
| Glu | Asp |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Val | Ile, Leu |

In an embodiment, the peptide antigen may be 5 to 120 amino acids in length, 5 to 100 amino acids in length, 5 to 75 amino acids in length, 5 to 50 amino acids in length, or 5 to 30 amino acids in length. In an embodiment, the peptide antigen may be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length.

In an embodiment, the peptide antigen is 20 to 30 amino acids in length. In an embodiment, the peptide antigen is 27 amino acids in length.

In an embodiment, the peptide antigen comprises at least one B cell epitope, at least one CTL epitope or any combination thereof.

B cell epitopes are epitopes recognized by B cells and by antibodies. B cell peptide epitopes are typically at least five amino acids, more often at least six amino acids, still more often at least seven or eight amino acids in length, and may be continuous (“linear”) or discontinuous (“conformational”); the latter being formed, for example, by the folding of a protein to bring non-contiguous parts of the primary amino acid sequence into physical proximity.

CTL epitopes are molecules recognized by cytotoxic T lymphocytes. CTL epitopes are typically presented on the surface of an antigen-presenting cell, complexed with MHC molecules. As used herein, the term “CTL epitope” refers to a peptide which is substantially the same as a natural CTL epitope of an antigen. The CTL epitope may be modified as compared to its natural counterpart, such as by one or two amino acids. Unless otherwise stated, reference herein to a CTL epitope is to an unbound molecule that is capable of being taken up by cells and presented on the surface of an antigen-presenting cell.

The CTL epitope should typically be one that is amendable to recognition by T cell receptors so that a cell-mediated immune response can occur. For peptides, CTL epitopes may interact with class I or class II MHC molecules. CTL epitopes presented by MHC class I molecules are typically peptides between 8 and 15 amino acids in length, and more often between 9 and 11 amino acids in length. CTL epitopes presented by MHC class II molecules are typically peptides between 5 and 24 amino acids in length, and more often between 13 and 17 amino acids in length. If the antigen is larger than these sizes, it will be processed by the immune system into fragments of a size more suitable for interaction with MHC class I or II molecules. Therefore, CTL epitopes may be part of larger peptide antigen than those mentioned above.

Many CTL epitopes are known. Several techniques of identifying additional CTL epitopes are recognized in the art. In general, these involve preparing a molecule which potentially provides a CTL epitope and characterizing the immune response to that molecule.

In an embodiment, the peptide antigen may be one that is associated with cancer, an infectious disease, an addiction disease, or any other disease or disorder.

Viruses, or parts thereof, from which a peptide antigen may be derived include for example, and without limitation, Cowpoxvirus, Vaccinia virus, Pseudocowpox virus, herpes virus, Human herpesvirus 1, Human herpesvirus 2, Cytomegalovirus, Human adenovirus A-F, Polyomavirus, human papillomavirus (HPV), Parvovirus, Hepatitis A virus, Hepatitis B virus, Hepatitis C virus, human immunodeficiency virus (HIV), Seneca Valley virus (SVV), Orthoreovirus, Rotavirus, Ebola virus, parainfluenza virus, influenza virus (e.g. H5N1 influenza virus, influenza A virus, influenza B virus, influenza C virus), Measles virus, Mumps virus, Rubella virus, Pneumovirus, respiratory syncytial virus, respiratory syncytial virus (RSV), Rabies virus, California encephalitis virus, Japanese encephalitis virus, Hantaan virus, Lymphocytic choriomeningitis virus, Coronavirus, Enterovirus, Rhinovirus, Poliovirus, Norovirus, Flavivirus, Dengue virus, West Nile virus, Yellow fever virus and varicella.

In an embodiment, the peptide antigen is derived from HPV. In an embodiment, the HPV peptide antigen is one that is associated with HPV-related cervical cancer or HPV-related head and neck cancer. In an embodiment, the peptide antigen is a peptide comprising the sequence RAHYNIVTF (HPV16E7 (H-2Db) peptide 49-57; R9F; SEQ ID NO: 9). In an embodiment, the peptide antigen is a peptide comprising the sequence YMLNLGPET (HPV Y9T peptide; SEQ ID NO: 10).

In an embodiment, the peptide antigen is derived from HIV. In an embodiment, the HIV peptide antigen may be derived from the V3 loop of HIV-1 gp120. In an embodiment, the HIV peptide antigen may be RGP10 (RGPGRAFVTI; SEQ ID NO: 11). RGP10 may be purchased from Genscript (Piscataway, N.J.). In another embodiment, the peptide antigen may be AMQ9 (AMQMLKETI; SEQ ID NO: 12). AMQ9 peptide is the immunodominant MHC class I epitope of gag for mice of the H-2Kd haplotype. AMQ9 may also be purchased from Genscript.

In an embodiment, the peptide antigen is derived from RSV. The RSV virion, a member of the genus Paramyxoviridae, is composed of a single strand of negative-sense RNA with 15,222 nucleotides. The nucleotides encode three transmembrane surface proteins (F, G and small hydrophobic protein or SH), two matrix proteins (M and M2), three nucleocapsid proteins (N, P and L), and two non-structural proteins (NS1 and NS2). In an embodiment, the peptide antigen may be derived from any one or more of the RSV proteins. In a particular embodiment, the peptide antigen may be derived from the SH protein of RSV or any other paramyxovirus, or a fragment thereof. The RSV peptide antigen may be any one or more of the RSV peptides described or disclosed in WO 2012/065997.

The SH protein, present in a number of paramyxoviruses (Collins 1990), is a transmembrane protein with an ectodomain or “extracellular” component. The human RSV SH protein contains 64 amino acids (Subgroup A) and 65 amino acids (Subgroup B) and is highly conserved.

Human RSV SH(Subgroup A):
(SEQ ID NO: 13)
MENTSITIEFSSKFWPYFTLIHMITTIISLLIIISIMIAILNKLCEY
NVFHNKTFELPRARVNT
Human RSV SH(Subgroup B):
(SEQ ID NO: 14)
MGNTSITIEFTSKFWPYFTLIHMILTLISLLIIITIMIAILNKLSEH
KTFCNKTLEQGQMYQINT

In an embodiment, the peptide antigen comprises or consists of the ectodomain of the SH protein (SHe) of a paramyxovirus, or a fragment or modified variant thereof. In an embodiment, SHe is derived from bovine RSV. In another embodiment, SHe is derived from a subgroup A human RSV strain or a subgroup B human RSV strain.

Subgroup A human RSV SHe(RSV SHe A):
(SEQ ID NO: 2)
NKLCEYNVFHNKTFELPRARVNT
Subgroup B human RSV SHe(RSV SHe B):
(SEQ ID NO: 3)
NKLSEHKTFCNKTLEQGQMYQINT

In an embodiment, the RSV peptide antigen may be in monomeric form, dimeric form, or another oligomeric form, or any combination thereof. In an embodiment, the peptide antigen comprising SHe A and/or SHe B is a monomer (e.g. a single polypeptide). In another embodiment, the peptide antigen comprising SHe A and/or SHe B is dimer (e.g. two separate polypeptides dimerized). Means of dimerization are known in the art. An exemplary procedure is to dissolve the RSV SHe peptide antigens in a mixture of 10% DMSO/0.5% acetic acid in water (w/w) and heat at 37° C. overnight.

In an embodiment, the peptide antigen derived from RSV may comprise or consist of any one or more of the following:

| | | SEQ |
|---|---|---|
| | | ID |
| Name | Sequence | NO |
| SheA | NKLCEYNVFHNKTFELPRARVNT | 2 |
| (mon- | | |
| omer) | | |
| SheA | NKLCEYNVFHNKTFELPRARVNT | 2 |
| (di- | \| | |
| mer) | NKLCEYNVFHNKTFELPRARVNT | 2 |
| SHeA | NKLSEYNVFHNKTFELPRARVNT | 15 |
| (C45S) | | |
| bSheA | NKLCDLNDHHTNSLDIRTRLRNDTQLITRAHEGSINQSSN | 16 |
| (mon- | | |
| omer) | | |
| bSheA | NKLCDLNDHHTNSLDIRTRLRNDTQLITRAHEGSINQSSN | 16 |
| (di- | \| | |
| mer) | NKLCDLNDHHTNSLDIRTRLRNDTQLITRAHEGSINQSSN | 16 |
| bSHeA | NKLSDLNDHHTNSLDIRTRLRNDTQLITRAHEGSINQSSN | 17 |
| (C45S) | | |
| SheB | NKLSEHKTFCNKTLEQGQMYQINT | 3 |
| (mon- | | |
| omer) | | |
| SheB | NKLSEHKTFCNKTLEQGQMYQINT | 3 |
| (di- | \| | |
| mer) | NKLSEHKTFCNKTLEQGQMYQINT | 3 |
| SHeB | NKLSEHKTFSNKTLEQGQMYQINT | 18 |
| (C51S) | | |
| SHeB | NKLCEHKTFSNKTLEQGQMYQINT | 19 |
| (C45S) | | |
| SHe B | NKLCEHKTFSNKTLEQGQMYQINT | 19 |
| (C45S) | \| | |
| | NKLCEHKTFSNKTLEQGQMYQINT | 19 |
| L-SHe | CGGGSNKLSEHKTFSNKTLEQGQMYQINT | 20 |
| B | \| | |
| (C51S) | CGGGSNKLSEHKTFSNKTLEQGQMYQINT | 20 |

As described for example in WO 2012/065997, the SHe peptide antigen may be genetically or chemically linked to a carrier. Exemplary embodiments of carriers suitable for presentation of peptide antigens are known in the art, some of which are described in WO 2012/065997. In another embodiment, the SHe peptide antigen may be linked to a sized lipid vesicle particle as described herein or a structure formed therefrom or resulting therefrom as a result of the methods of manufacture.

In another embodiment, the peptide antigen is derived from an influenza virus. Influenza is a single-stranded RNA virus of the family Orthomyxoviridae and is often characterized based on two large glycoproteins on the outside of the viral particle, hemagglutinin (HA) and neuraminidase (NA). Numerous HA subtypes of influenza A have been identified (Kawaoka 1990; Webster 1983). In some embodiments, the antigen may be derived from the HA or NA glycoproteins. In a particular embodiment, the antigen may be recombinant HA antigen (H5N1, A/Vietnam/1203/2004; Protein Sciences; USA), such as derived from the sequence found under GenBank Accession number AY818135 or any suitable sequence variant thereof.

Bacteria, or parts thereof, from which a peptide antigen may be derived include for example, and without limitation, Anthrax (Bacillus anthracis), Brucella, Bordetella pertussis, Candida, Chlamydia pneumoniae, Chlamydia psittaci, Cholera, Clostridium botulinum, Coccidioides immitis, Cryptococcus, Diphtheria, Escherichia coli O157: H7, Enterohemorrhagic Escherichia coli, Enterotoxigenic Escherichia coli, Haemophilus influenzae, Helicobacter pylori, Legionella, Leptospira, Listeria, Meningococcus, Mycoplasma pneumoniae, Mycobacterium, Pertussis, Pneumonia, Salmonella, Shigella, Staphylococcus, Streptococcus pneumoniae and Yersinia enterocolitica.

In an embodiment, the peptide antigen is derived from a Bacillus anthracis. Without limitation, the peptide antigen may for example be derived from anthrax recombinant protective antigen (rPA) (List Biological Laboratories, Inc.; Campbell, Calif.) or anthrax mutant recombinant protective antigen (mrPA). rPA has an approximate molecular weight of 83,000 daltons (Da) and corresponds a cell binding component of the three-protein exotoxin produced by Bacillus anthracis. The protective antigen mediates the entry of anthrax lethal factor and edema factor into the target cell. In some embodiments the antigen may be derived from the sequence found under GenBank Accession number P13423, or any suitable sequence variant thereof.

Protozoa, or parts thereof, from which a peptide antigen may be derived include for example, and without limitation, the genus Plasmodium (Plasmodium falciparum, Plasmodium malariae, Plasmodium vivax, Plasmodium ovale or Plasmodium knowlesi), which causes malaria.

In an embodiment, the peptide antigen is derived from a Plasmodium species. For example, and without limitation, the peptide antigen may be derived from the circumsporozoite protein (CSP), which is a secreted protein of the sporozoite stage of the malaria parasite (Plasmodium sp.). The amino-acid sequence of CSP consists of an immunodominant central repeat region flanked by conserved motifs at the N- and C-termini that are implicated in protein processing as the parasite travels from the mosquito to the mammalian vector. The structure and function of CSP is highly conserved across the various strains of malaria that infect humans, non-human primates and rodents. In an embodiment, the peptide antigen derived from CSP is a malaria virus-like particle (VLP) antigen which comprises circumsporozoite T and B cell epitopes displayed on the woodchuck hepatitis virus core antigen.

In another embodiment, the peptide antigen may be derived from a cancer or tumor-associated protein, such as for example, a membrane surface-bound cancer antigen.

In an embodiment, the cancer may be one that is caused by a pathogen, such as a virus. Viruses linked to the development of cancer are known to the skilled person and include, but are not limited to, human papillomaviruses (HPV), John Cunningham virus (JCV), Human herpes virus 8, Epstein Barr Virus (EBV), Merkel cell polyomavirus, Hepatitis C Virus and Human T cell leukaemia virus-1. Thus, in an embodiment, the peptide antigen may be derived from a virus that is linked to the development of cancer.

In an embodiment, the peptide antigen is a cancer-associated antigen. Many cancer or tumor-associated proteins are known in the art such as for example, and without limitation, those described in WO 2016/176761. The methods, dried preparations, compositions, uses and kits disclosed herein may use or comprise any peptide antigen of a cancer-associated antigen, or a fragment or modified variant thereof.

In a particular embodiment, the peptide antigen is one or more survivin antigens.

Survivin, also called baculoviral inhibitor of apoptosis repeat-containing 5 (BIRC5), is a protein involved in the negative regulation of apoptosis. It has been classed as a member of the family of inhibitors of apoptosis proteins (IAPB). Survivin is a 16.5 kDa cytoplasmic protein containing a single BIR motif and a highly charged carboxy-terminal coiled region instead of a RING finger. The gene coding for survivin is nearly identical to the sequence of Effector Cell Protease Receptor-1 (EPR-1), but oriented in the opposite direction. The coding sequence for the survivin (Homo sapiens) is 429 nucleotides long including stop codons:

SEQ ID NO: 21
atgggtgccc cgacgttgcc ccctgcctgg cagccctttc tcaaggacca ccgcatctct 60
acattcaaga actggccctt cttggagggc tgcgcctgca ccccggagcg gatggccgag 120
gctggcttca tccactgccc cactgagaac gagccagact tggcccagtg tttcttctgc 180
ttcaaggagc tggaaggctg ggagccagat gacgacccca tagaggaaca taaaaagcat 240
tcgtccggtt gcgctttcct ttctgtcaag aagcagtttg aagaattaac ccttggtgaa 300
tttttgaaac tggacagaga aagagccaag aacaaaattg caaaggaaac caacaataag 360
aagaaagaat ttgaggaaac tgcgaagaaa gtgcgccgtg ccatcgagca gctggctgcc 420
atggattga 429

The encoded protein survivin (Homo sapiens) is 142 amino acids long:

```
SEQ ID NO: 22
Met Gly Ala Pro Thr Leu Pro Pro Ala Trp Gln Pro
1 5 10
Phe Leu Lys Asp His Arg Ile Ser Thr Phe Lys Asn
                15 20
Trp Pro Phe Leu Glu Gly Cys Ala Cys Thr Pro Glu
25 30 35
Arg Met Ala Glu Ala Gly Phe Ile His Cys Pro Thr
                        40 45
Glu Asn Glu Pro Asp Leu Ala Gln Cys Phe Phe Cys
        50 55 60
Phe Lys Glu Leu Glu Gly Trp Glu Pro Asp Asp Asp
                                65 70
Pro Ile Glu Glu His Lys Lys His Ser Ser Gly Cys
                75 80
Ala Phe Leu Ser Val Lys Lys Gln Phe Glu Glu Leu
85 90 95
Thr Leu Gly Glu Phe Leu Lys Leu Asp Arg Glu Arg
                        100 105
Ala Lys Asn Lys Ile Ala Lys Glu Thr Asn Asn Lys
        110 115 120
Lys Lys Glu Phe Glu Glu Thr Ala Lys Lys Val Arg
                                125 130
Arg Ala Ile Glu Gln Leu Ala Ala Met Asp
        135 140
```

In an embodiment, the peptide antigen is any peptide, polypeptide or variant thereof derived from a survivin protein, or a fragment thereof.

In an embodiment, the peptide antigen may be a survivin antigen, such as for example and without limitation, those disclosed in WO 2016/176761.

In an embodiment, the survivin peptide antigen may comprise the full length survivin polypeptide. Alternatively, the survivin peptide antigen may be a survivin peptide comprising a fragment of any length of the survivin protein. Exemplary embodiments include a survivin peptide that comprises at least 5, 6, 7, 8, 9, 10, 11, 12, 13, 14, 15, 16, 17, 18, 19 or 20 amino acid residues. In specific embodiments, the survivin peptide consists of a heptapeptide, an octapeptide, a nonapeptide, a decapeptide or an undecapeptide, consisting of 7, 8, 9, 10, 11 consecutive amino acid residues of the survivin protein (e.g. SEQ ID NO: 22), respectively. Particular embodiments of the survivin antigen include survivin peptides of about 9 or 10 amino acids.

Survivin peptide antigens also encompass variants and functional equivalents of natural survivin peptides. Variants or functional equivalents of a survivin peptide encompass peptides that exhibit amino acid sequences with differences as compared to the specific sequence of the survivin protein, such as one or more amino acid substitutions, deletions or additions, or any combination thereof. The difference may be measured as a reduction in identity as between the survivin protein sequence and the survivin peptide variant or survivin peptide functional equivalent.

In an embodiment, a vaccine composition of the invention may include any one or more of the survivin peptides, survivin peptide variants or survivin peptide functional equivalents disclosed in WO 2004/067023; WO 2006/081826 or WO 2016/176761.

In a particular embodiment, the survivin peptide antigen may be any one or more of:

i) [HLA-A1]
(SEQ ID NO: 23)
FEELTLGEF
ii) [HLA-A1]
(SEQ ID NO: 4)
FTELTLGEF
iii) [HLA-A2]
(SEQ ID NO: 24)
LTLGEFLKL
iv) [HLA-A2]
(SEQ ID NO: 5)
LMLGEFLKL
v) [HLA-A3]
(SEQ ID NO: 25)
RISTFKNWPF
vi) [HLA-A3]
(SEQ ID NO: 6)
RISTFKNWPK
vii) [HLA-A24]
(SEQ ID NO: 7)
STFKNWPFL
viii) [HLA-B7]
(SEQ ID NO: 8)
LPPAWQPFL

The above-listed survivin peptides represent, without limitation, exemplary MEW Class I restricted peptides. The specific MEW Class I HLA molecule to which each of the survivin peptides is believed to bind is shown on the right in square brackets.

In an embodiment, the methods, dried preparations, compositions, uses and kits disclosed herein use or comprise one or more of the following survivin peptide antigens:

i) [HLA-A1]
(SEQ ID NO: 4)
FTELTLGEF
ii) [HLA-A2]
(SEQ ID NO: 5)
LMLGEFLKL
iii) [HLA-A3]
(SEQ ID NO: 6)
RISTFKNWPK
iv) [HLA-A24]
(SEQ ID NO: 7)
STFKNWPFL
v) [HLA-B7]
(SEQ ID NO: 8)
LPPAWQPFL

In an embodiment, the methods, dried preparations, compositions, uses and kits disclosed herein use or comprise all five of the survivin peptide antigens listed above.

In an embodiment, the peptide antigen is a self-antigen. As is well-known in the art, a self-antigen is an antigen that originates from within the body of a subject. The immune system is usually non-reactive against self-antigens under normal homeostatic conditions. These types of antigens therefore pose a difficulty in the development of targeted immune therapies. In an embodiment, the peptide antigen is a self-antigen or a fragment or modified variant thereof.

In an embodiment, the peptide antigen is a neoantigen. As used herein, the term “neoantigen” refers to a class of tumor antigens which arise from tumor-specific mutations in an expressed protein. The neoantigen can be derived from any cancer, tumor or cell thereof.

In the context of neoantigens, the term “derived from” as used herein encompasses, without limitation: a neoantigen that is isolated or obtained directly from an originating source (e.g. a subject); a synthetic or recombinantly generated neoantigen that is identical in sequence to a neoantigen from an originating source; or a neoantigen which is made from a neoantigen of an originating source or a fragment thereof.

The mutations in the expressed protein that create the neoantigen may be patient-specific. By “patient-specific”, it is meant that the mutation(s) are unique to an individual subject. However, it is possible that more than one subject will share the same mutation(s). Thus, a “patient-specific” mutation may be shared by a small or large sub-population of subjects.

The neoantigen may comprise one or more neoepitopes. As used herein, the term “epitope” refers to a peptide sequence which can be recognized by the immune system, specifically by antibodies, B cells or T cells. A “neoepitope” is an epitope of a neoantigen which comprises a tumor-specific mutation as compared to the native amino acid sequence. Generally, neoepitopes may be identified by screening neoantigens for anchor residues that have the potential to bind patient HLA. The neoepitopes are normally ranked using algorithms, such as NetMHC, that can predict peptide binding to HLA.

A “T-cell neoepitope” is to be understood as meaning a mutated peptide sequence which can be bound by the MEW molecules of class I or II in the form of a peptide-presenting MEW molecule or MEW complex. The T-cell neoepitope should typically be one that is amenable to recognition by T cell receptors so that a cell-mediated immune response can occur. A “B-cell neoepitope” is to be understood as meaning a mutated peptide sequence which can be recognized by B cells and/or by antibodies.

In some embodiments, at least one of the neoepitopes of the neoantigen is a patient-specific neoepitope. As used herein, by “patient-specific neoepitope”, it is meant that the mutation(s) in the neoepitope are unique to an individual subject. However, it is possible that more than one subject will share the same mutation(s). Thus, a “patient-specific neoepitope” may be shared by a small or large sub-population of subjects.

As is apparent from the above, neoantigens can comprise a diverse set of peptides that are unique to an individual. These peptides may have different solubility properties which would make them difficult to formulate in conventional types of vaccine formulations, such as aqueous buffer or emulsion type formulations. Additionally, there may be pre-existing tolerance to these peptides in the host from which they were derived. These aspects, among others, may cause the neoantigens to be weakly immunogenic. Therefore, it is important to deliver them in a composition that is capable of generating a robust immune response, as disclosed herein.

As used herein, by “weakly immunogenic” it is meant that in conventional vaccines (e.g. aqueous vaccines, emulsions, etc.), the neoantigens have little or no ability to induce, maintain and/or boost a neoantigen-specific immune response. In an embodiment, a weakly immunogenic neoantigen is one that has little or no ability to induce, maintain and/or boost a neoantigen-specific immune response after a single administration of the neoantigen.

In an embodiment, the neoantigen may be selected from mutated somatic proteins of a cancer using selection algorithms such as NetMHC which look for motifs predicted to bind to MHC class I and/or MHC class II proteins.

In an embodiment, the neoantigen may be derived from a mutated gene or protein that has previously been associated with cancer phenotypes, such as for example tumor suppressor genes (e.g. p53); DNA repair pathway proteins (e.g. BRCA2) and oncogenes. Exemplary embodiments of genes which often contain mutations giving rise to cancer phenotypes are described, for example, in Castle 2012. The skilled person will be well aware of other mutated genes and/or proteins associated with cancer, and these are available from other literature sources.

In some embodiments, the neoantigen may comprise or consist of the neoantigens disclosed by Castle 2012. Castle 2012 does not provide the actual sequences of the neoantigens, but does provide the gene ID and location of the mutated peptide from which the actual sequence can be identified using e.g. the PubMed database available online from the National Center for Biotechnology Information (NCBI).

In an embodiment, the neoantigen may be one or more of the Mut1-50 neoantigens disclosed in Table 1 of Castle 2012, or a neoantigen of the same or related protein (e.g. a human homologue). In an embodiment, the neoantigen may be selected from the neoantigen peptides listed in Table 5 herein, or a neoantigen of the same or related protein (e.g. a human homologue). In an embodiment, the neoantigen may be one or more of Mut25 (STANYNTSHLNNDVWQIFENPVDWKEK; SEQ ID NO: 26), Mut30 (PSKPSFQEFVDWENVSPELNSTDQPFL; SEQ ID NO: 27) and Mut44 (EFKHIKAFDRTFANNPGPMVVFATPGM; SEQ ID NO: 28), or a neoantigen of the same or related protein (e.g. a human homologue).

In an embodiment, the methods disclosed herein may be used to prepare a pharmaceutical composition comprising a mixture of multiple different neoantigens in a single composition. In an embodiment, the methods disclosed herein are for formulating a mixture of mixture of 5, 6, 7, 8, 9, 10, 11, 12, 13, 14, 15, 16, 17, 18, 19, 20, 21, 22, 23, 24, 25, 26, 27, 28, 29, 30 or more different neoantigens in a single composition. In an embodiment, the methods disclosed herein are for formulating a mixture of 10-20 different neoantigens in a single composition. In an embodiment, each of the different neoantigens is 20 to 30 amino acids in length. As described herein, the disclosed methods are capable of providing a stable composition comprising these multiple different neoantigens, without significant steps of neoantigen selection.

DNA or RNA Polynucleotides that Encodes a Polypeptide

In an embodiment, the therapeutic agent may be a DNA polynucleotide or RNA polynucleotide that encodes a polypeptide. In an embodiment, the DNA or RNA polynucleotide encodes one or more of the peptide antigens described herein.

As used herein, the “DNA or RNA polynucleotide” encompasses a chain of nucleotides of any length (e.g. 9, 12, 15, 18, 21, 24, 27, 30, 60, 90, 120, 150, 300, 600, 1200, 1500 or more nucleotides) or number of strands (e.g. single-stranded or double-stranded). Polynucleotides may be DNA (e.g. genomic DNA, cDNA, plasmid DNA) or RNA (e.g. mRNA) or combinations thereof. The polynucleotide may be naturally occurring or synthetic (e.g. chemically synthesized). It is contemplated that the polynucleotide may contain modifications of one or more nitrogenous bases, pentose sugars or phosphate groups in the nucleotide chain. Such modifications are well-known in the art and may be for the purpose of e.g. improving stability, solubility or transcriptional/translational activity of the polynucleotide.

In an embodiment, the polynucleotide encodes a polypeptide to be expressed in vivo in a subject. The invention is not limited to the expression of any particular type of polypeptide.

The polynucleotide may be used in various forms. In an embodiment, a naked polynucleotide may be used, either in linear form, or inserted into a plasmid, such as an expression plasmid. In other embodiments, a live vector such as a viral vector or bacterial vector may be used.

Depending on the nature of the polynucleotide and the intended use, one or more regulatory sequences that aid in transcription of DNA into RNA and/or translation of RNA into a polypeptide may be present. For example, if it is intended or not required that the polynucleotide be transcribed or translated, such regulatory sequences may be absent. In some instances, such as in the case of a polynucleotide that is a messenger RNA (mRNA) molecule, regulatory sequences relating to the transcription process (e.g. a promoter) are not required, and protein expression may be effected in the absence of a promoter. The skilled artisan can include suitable regulatory sequences as the circumstances require.

In some embodiments, the polynucleotide is present in an expression cassette, in which it is operably linked to regulatory sequences that will permit the polynucleotide to be expressed in the subject. The choice of expression cassette depends on the subject as well as the features desired for the expressed polypeptide.

Typically, an expression cassette includes a promoter that is functional in the subject and can be constitutive or inducible; a ribosome binding site; a start codon (ATG) if necessary; the polynucleotide encoding the polypeptide of interest; a stop codon; and optionally a 3′ terminal region (translation and/or transcription terminator). Additional sequences such as a region encoding a signal peptide may be included. The polynucleotide encoding the polypeptide of interest may be homologous or heterologous to any of the other regulatory sequences in the expression cassette. Sequences to be expressed together with the polypeptide of interest, such as a signal peptide encoding region, are typically located adjacent to the polynucleotide encoding the protein to be expressed and placed in proper reading frame. The open reading frame constituted by the polynucleotide encoding the protein to be expressed solely or together with any other sequence to be expressed (e.g. the signal peptide), is placed under the control of the promoter so that transcription and translation occur in the subject to which the composition is administered.

Promoters suitable for expression of polynucleotides in a wide range of host systems are well-known in the art. Promoters suitable for expression of polynucleotides in mammals include those that function constitutively, ubiquitously or tissue-specifically. Examples of non-tissue specific promoters include promoters of viral origin. Examples of viral promoters include Mouse Mammary Tumor Virus (MMTV) promoter, Human Immunodeficiency Virus Long Terminal Repeat (HIV LTR) promoter, Moloney virus, avian leukosis virus (ALV), Cytomegalovirus (CMV) immediate early promoter/enhancer, Rous Sarcoma Virus (RSV), adeno-associated virus (AAV) promoters; adenoviral promoters, and Epstein Barr Virus (EBV) promoters. Compatibility of viral promoters with certain polypeptides is a consideration since their combination may affect expression levels. It is possible to use synthetic promoter/enhancers to optimize expression (see e.g. US patent publication 2004/0171573).

An example of a tissue-specific promoter is the desmin promoter which drives expression in muscle cells (Li 1989; Li & Paulin 1991; and Li & Paulin 1993). Other examples include artificial promoters such as a synthetic muscle specific promoter and a chimeric muscle-specific/CMV promoter (Li 1999; Hagstrom 2000).

As noted above, the polynucleotide of interest, together with any necessary regulatory sequences, may be delivered naked, e.g. either alone or as part of a plasmid, or may be delivered in a viral or bacterial or bacterial vector.

Whether a plasmid-type vector, or a bacterial or viral vector is used, it may be desirable that the vector be unable to replicate or integrate substantially in the subject. Such vectors include those whose sequences are free of regions of substantial identity to the genome of the subject, as to minimize the risk of host-vector recombination. One way to do this is to use promoters not derived from the recipient genome to drive expression of the polypeptide of interest. For example, if the recipient is a mammal, the promoter is preferably non-mammalian derived though it should be able to function in mammalian cells, e.g. a viral promoter.

Viral vectors that may be used to deliver the polynucleotide include e.g. adenoviruses and poxviruses. Useful bacterial vectors include e.g. Shigella, Salmonella, Vibrio cholerae, Lactobacillus, Bacille bilie de Calmette-Guerin (BCG), and Streptococcus.

An example of an adenovirus vector, as well as a method for constructing an adenovirus vector capable of expressing a polynucleotide, is described in U.S. Pat. No. 4,920,209. Poxvirus vectors include vaccinia and canary pox virus, described in U.S. Pat. Nos. 4,722,848 and 5,364,773, respectively. Also see, e.g., Tartaglia 1992 for a description of a vaccinia virus vector and Taylor 1995 for a reference of a canary pox.

Poxvirus vectors capable of expressing a polynucleotide of interest may be obtained by homologous recombination as described in Kieny 1984, so that the polynucleotide is inserted in the viral genome under appropriate conditions for expression in mammalian cells.

With respect to bacterial vectors, non-toxicogenic Vibrio cholerae mutant strains that are useful for expressing a foreign polynucleotide in a host are known. Mekalanos 1983 and U.S. Pat. No. 4,882,278 describe strains which have a substantial amount of the coding sequence of each of the two ctxA alleles deleted so that no functional cholerae toxin is produced. WO 92/11354 describes a strain in which the irgA locus is inactivated by mutation; this mutation can be combined in a single strain with ctxA mutations. WO 94/01533 describes a deletion mutant lacking functional ctxA and attRSl DNA sequences. These mutant strains are genetically engineered to express heterologous proteins, as described in WO 94/19482.

Attenuated Salmonella typhimurium strains, genetically engineered for recombinant expression of heterologous proteins are described in Nakayama 1988 and WO 92/11361.

Other bacterial strains which may be used as vectors to express a foreign protein in a subject are described for Shigella flexneri in High 1992 and Sizemore 1995; for Streptococcus gordonii in Medaglini 1995; and for Bacille Calmette Guerin in Flynn 1994, WO 88/06626, WO 90/00594, WO 91/13157, WO 92/01796, and WO 92/21376.

In bacterial vectors, the polynucleotide of interest may be inserted into the bacterial genome or remain in a free state as part of a plasmid.

Hormones

In an embodiment, the therapeutic agent may be a hormone, or a fragment, analog or variant thereof. The hormone, fragment, analog or variant thereof may be obtained from a natural source or be synthetically prepared.

Exemplary hormones include, without limitation, amylin, insulin, glucagon, erythropoietin (EPO), glucagon-like peptide-1 (GLP-1), melanocyte stimulating hormone (MSH), parathyroid hormone (PTH), thyroid-stimulating hormone, growth hormone (GH), growth hormone releasing hormone (GHRH), calcitonin, somatostatin, somatomedin (insulin-like growth factor), interleukins (e.g. interleukins 1-17), granulocyte/monocyte colony stimulating factor (GM-CSF), granulocyte colony stimulating factor (G-CSF), testosterone, interferons (e.g. interferon-alfa or -gamma), leptin, luteinizing hormone (LH), follicle-stimulating hormone (FSH), human chorionic gonadotropin (hCG), enkephalin, basic fibroblast growth factor (bFGF), luteinizing hormone, gonadotropin releasing hormone (GnRH), brain-derived natriuretic peptide (BNP), tissue plasminogen activator (TPA), oxytocin, relaxin, steroids (e.g. androgens, estrogens, glucocorticoids, progestogens and secosteroids) and analogs and combinations thereof

Cytokines

In an embodiment, the therapeutic agent may be a cytokine, or a fragment, analog or variant thereof. The cytokine, fragment, analog or variant thereof may be obtained from a natural source or be synthetically prepared.

Exemplary cytokines include, without limitation, chemokines, interferons, interleukins, lymphokines and tumor necrosis factors, and analogs thereof.

Allergens

In an embodiment, the therapeutic agent may be an allergen, or a fragment, analog or variant thereof. The allergen, fragment, analog or variant thereof may be obtained from a natural source or be synthetically prepared.

An “allergen”, as used herein, refers to any substance that can cause an allergy. The allergen may be derived from, without limitation, cells, cell extracts, proteins, polypeptides, peptides, polysaccharides, polysaccharide conjugates, peptide and non-peptide mimics of polysaccharides and other molecules, small molecules, lipids, glycolipids, and carbohydrates of plants, animals, fungi, insects, food, drugs, dust, and mites. Allergens include but are not limited to environmental aeroallergens; plant pollens (e.g. ragweed/hayfever); weed pollen allergens; grass pollen allergens; Johnson grass; tree pollen allergens; ryegrass; arachnid allergens (e.g. house dust mite allergens); storage mite allergens; Japanese cedar pollen/hay fever; mold/fungal spore allergens; animal allergens (e.g. dog, guinea pig, hamster, gerbil, rat, mouse, etc., allergens); food allergens (e.g. crustaceans; nuts; citrus fruits; flour; coffee); insect allergens (e.g. fleas, cockroach); venoms: (Hymenoptera, yellow jacket, honey bee, wasp, hornet, fire ant); bacterial allergens (e.g. streptococcal antigens; parasite allergens such as Ascaris antigen); viral allergens; drug allergens (e.g. penicillin); hormones (e.g. insulin); enzymes (e.g. streptokinase); and drugs or chemicals capable of acting as incomplete antigens or haptens (e.g. the acid anhydrides and the isocyanates).

Catalytic DNA or RNA

In an embodiment, the therapeutic agent may be a catalytic DNA (deoxyribozyme) or a catalytic RNA (ribozyme).

As used herein, the term “catalytic DNA” refers to any DNA molecule with enzymatic activity. In an embodiment, the catalytic DNA is a single-stranded DNA molecule. In an embodiment, the catalytic DNA is synthetically produced as opposed to naturally occurring.

The catalytic DNA may perform one or more chemical reactions. In an embodiment, the catalytic DNA is a ribonuclease, whereby the catalytic DNA catalyzes the cleavage of ribonucleotide phosphodiester bonds. In another embodiment, the catalytic DNA is a DNA ligase, whereby the catalytic DNA catalyzes the joining of two polynucleotide molecules by forming a new bond. In other embodiments, the catalytic DNA can catalyze DNA phosphorylation, DNA adenylation, DNA deglycosylation, porphyrin metalation, thymine dimer photoreversion, or DNA cleavage.

As used herein, the term “catalytic RNA” refers to any RNA molecule with enzymatic activity. Catalytic RNAs are involved in a number of biological processes, including RNA processing and protein synthesis. In an embodiment, the catalytic RNA is a naturally occurring RNA. In an embodiment, the catalytic RNA is synthetically produced.

Antisense RNA

In an embodiment, the therapeutic agent may be an antisense RNA.

As used herein, an “antisense RNA” is any single-stranded RNA that is complementary to a messenger RNA (mRNA). The antisense RNA may exhibit 100% complementarity to the mRNA or less than 100% complementarity so long as the antisense RNA is still able to inhibit translation of the mRNA by base pairing to it, thereby obstructing the translation machinery.

In an embodiment, the antisense RNA is highly structured, comprised of one or more stem-and-loop secondary structures, flanked or separated by single-stranded (unpaired) regions. In some embodiments, tertiary structures, such as pseudoknots, may form between two or more secondary structural elements.

Interfering RNA and Antagomirs

In an embodiment, the therapeutic agent may be an interfering RNA, such as a small interfering RNA (siRNA), a microRNA (miRNA) or a small hairpin RNA (shRNA).

RNA interference (RNAi) is a biological process in which RNA molecules inhibit gene expression or translation, by neutralizing targeted mRNA molecules. Two types of small ribonucleic acid (RNA) molecules—microRNA (miRNA) and small interfering RNA (siRNA) are central to RNA interference.

siRNA is a class of double-stranded RNA molecules that are typically 20-25 base pairs in length. It interferes with the expression of specific genes with complementary nucleotide sequences by degrading mRNA after transcription, thereby preventing translation. The natural structure of siRNA is typically a short 20-25 double-stranded RNA with two overhanging nucleotides on each end. The Dicer enzyme catalyzes production of siRNAs from long dsRNAs and small hairpin RNAs (shRNA). shRNA is an artificial RNA molecule with a tight hairpin turn. The design and production of siRNA molecules, and mechanisms of action, are known in the art.

miRNAs resemble siRNAs, except miRNAs derive from regions of RNA transcripts that fold back on themselves to form short hairpins, whereas siRNAs derive from longer double-stranded RNA.

In an embodiment, the therapeutic agent may be any one or more of these interfering RNAs (siRNA, miRNA or shRNA). The interfering RNA should be one which is capable of decreasing or silencing (preventing) the expression of a gene/mRNA of its endogenous cellular counterpart. In an embodiment, the interfering RNA derived from a naturally occurring interfering RNA. In an embodiment, the interfering RNA is synthetically produced.

In an embodiment, the therapeutic agent may be an antagomir. Antagomirs (also known as anti-miRs or blockmirs) are synthetically engineered oligonucleotides that silence endogenous miRNA. It is unclear how antagomirization (the process by which an antagomir inhibits miRNA activity) operates, but it is believed to inhibit by irreversibly binding the miRNA. Because of the promiscuity of microRNAs, antagomirs could affect the regulation of many different mRNA molecules. Antagomirs are designed to have a sequence that is complementary to an mRNA sequence that serves as a binding site for microRNA.

Drugs

In an embodiment, the therapeutic agent is a drug, i.e. a chemical substance used to treat, cure, prevent or diagnose a disease, disorder or condition.

In an embodiment, and without limitation, exemplary drugs include immunomodulatory agents (immunostimulants and immunosuppressives), an immune response checkpoint molecule, antipyretics, analgesics, anti-migraine agents, anti-coagulant agents, anti-emetic agents, anti-inflammatory agents, antiviral agents, antibacterial agents, anti-fungal agents, cardiovascular agents, central nervous system agents, anti-hypertensive and vasodilator agents, sedatives, narcotic agonists, chelating agents, anti-diuretic agents, and anti-cancer agents, anti-neoplastic agents. Examples include the following:

In an embodiment, the drug is a small molecule drug. As used herein, the term “small molecule drug” refers an organic compound that may be used to treat, cure, prevent or diagnose a disease, disorder or condition.

The term “small molecule” is understood to refer to a low molecular weight compound which may be synthetically produced or obtained from natural sources and typically has a molecular weight of less than 2000 Da, less than 1000 Da or less than 600 Da. In a particular embodiment, the small molecule has a molecular weight of less than 900 Da, which allows for the possibility to rapidly diffuse across cell membranes. More particularly the small molecule has a molecular weight of less than 600 Da, and even more particularly less than 500 Da.

In an embodiment, the small molecule drug has a molecule weight of between about 100 Da to about 2000 Da; about 100 Da to about 1500 Da; about 100 Da to about 1000 Da; about 100 Da to about 900 Da; about 100 Da to about 800 Da; about 100 Da to about 700 Da; about 100 Da to about 600 Da; or about 100 Da to about 500 Da. In an embodiment, the small molecule drug has a molecular weight of about 100 Da, about 150 Da, about 200 Da, about 250 Da, about 300 Da, about 350 Da, about 400 Da, about 450 Da, about 500 Da, about 550 Da, about 600 Da, about 650 Da, about 700 Da, about 750 Da, about 800 Da, about 850 Da, about 900 Da, about 950 Da or about 1000 Da. In an embodiment, the small molecule drug may have a size on the order of 1 nm.

In an embodiment, the small molecule drug is one or more of: Epacadostat, Rapamycin, Doxorubicin, Valproic acid, Mitoxantrone, Vorinostat, Cyclophosphamide, Irinotecan, Cisplatin or Methotrexate. In a particular embodiment, the small molecule drug is Cyclophosphamide.

In an embodiment, the small molecule drug is an agent that interferes with DNA replication. As used herein, the expression “interferes with DNA replication” is intended to encompass any action that prevents, inhibits or delays the biological process of copying (i.e., replicating) the DNA of a cell. The skilled person will appreciate that there exist various mechanisms for preventing, inhibiting or delaying DNA replication, such as for example DNA cross-linking, methylation of DNA, base substitution, etc. The present disclosure encompasses the use of any agent that interferes with DNA replication by any means known in the art. Exemplary, non-limiting embodiments of such agents are described, for example, in WO2014/153636 and/or in PCT/CA2017/050539. In an embodiment, the agent that interferes with DNA replication is an alkylating agent, such as for example a nitrogen mustard alkylating agent. In an embodiment, the agent that interferes with DNA replication is Cyclophosphamide.

In an embodiment, the small molecule drug is an immune response checkpoint inhibitor. As used herein, an “immune response checkpoint inhibitor” refers to any compound or molecule that totally or partially reduces, inhibits, interferes with or modulates one or more checkpoint proteins. Checkpoint proteins regulate T-cell activation or function. Numerous checkpoint proteins are known, such as for example CTLA-4 and its ligands CD80 and CD86; and PD-1 and its ligands PD-L1 and PD-L2. Checkpoint proteins are responsible for co-stimulatory or inhibitory interactions of T-cell responses. Checkpoint proteins regulate and maintain self-tolerance and the duration and amplitude of physiological immune responses. Herein, the term “immune response checkpoint inhibitor” may be used interchangeably with “checkpoint inhibitor”. Exemplary non-limiting embodiments of checkpoint inhibitors are hereinafter described.

In an embodiment, the immune response checkpoint inhibitor is an inhibitor of Programmed Death-Ligand 1 (PD-L1, also known as B7-H1, CD274), Programmed Death 1 (PD-1, CD279), CTLA-4 (CD154), PD-L2 (B7-DC, CD273), LAG3 (CD223), TIM3 (HAVCR2, CD366), 41BB (CD137), 2B4, A2aR, B7H1, B7H3, B7H4, BTLA, CD2, CD27, CD28, CD30, CD40, CD70, CD80, CD86, CD160, CD226, CD276, DR3, GAL9, GITR, HVEM, IDO1, IDO2, ICOS (inducible T cell costimulator), KIR, LAIR1, LIGHT, MARCO (macrophage receptor with collageneous structure), PS (phosphatidylserine), OX-40, SLAM, TIGIT, VISTA, VTCN1, or any combination thereof.

In an embodiment, the immune response checkpoint inhibitor is an inhibitor of PD-L1, PD-1, CTLA-4 or any combination thereof.

In an embodiment, the drug is a biologic drug. As used herein, a “biologic drug” is any pharmaceutical drug product manufactured in, extracted from, or semisynthesized from biological sources. In an embodiment, the biologic drug is a blood component, a cell, a cellular component, an allergen, an antibody, a gene or fragment thereof, a tissue, a tissue component, or a recombinant protein.

Antibodies

In an embodiment, the therapeutic agent is an antibody, an antigen binding fragment thereof or a derivative thereof.

“Antibody” as used herein means an antibody of classes IgG, IgM, IgA, IgD or IgE, or fragments, or derivatives thereof, including Fab, F(ab′)2, Fd, and single chain antibodies, diabodies, bispecific antibodies, bifunctional antibodies and derivatives thereof. The antibody can be an antibody isolated from the serum sample of mammal, a monoclonal antibody, a polyclonal antibody, an affinity purified antibody, or mixtures thereof which exhibit sufficient binding specificity to a desired epitope or a sequence derived therefrom.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

As used herein, the term “antigen-binding fragment” refers to any fragment or portion of an antibody, or variant thereof, that remains capable of binding a specific target antigen of the full length antibody. In an embodiment, the antigen-binding fragment comprises the heavy chain variable and/or light chain variable region of the antibody.

In an embodiment, the antibody can be an anti-PD-1 antibody, a variant thereof or an antigen-binding fragment thereof, or a combination thereof. In an embodiment, the PD-1 antibody may be Nivolumab (Opdivo™). In an embodiment, the PD-1 antibody may be pembrolizumab (Keytruda™).

In other embodiments, without limitation, the antibody may be an anti-PD1 or anti-PDL1 antibody, such as for example those disclosed in WO 2015/103602. For example, in an embodiment, the anti-PD-1 antibody or anti-PD-L1 antibody may be selected from: nivolumab, pembrolizumab, pidilizumab, BMS-936559 (see ClinicalTrials.gov; Identifier NCT02028403), MPDL3280A (Roche, see ClinicalTrials.gov; Identifier NCT02008227), MDX1105-01 (Bristol Myers Squibb, see ClinicalTrials.gov; Identifier NCT00729664), MEDI4736 (MedImmune, see ClinicalTrials.gov; Identifier NCT01693562), and MK-3475 (Merck, see ClinicalTrials.gov; Identifier NCT02129556). In an embodiment, the anti-PD-1 antibody may be RMP1-4 or J43 (BioXCell) or a human or humanized counterpart thereof.

In an embodiment, the antibody is an anti-CTL4 antibody, a variant thereof or an antigen-binding fragment thereof, or a combination thereof. The anti-CTL4 antibody can inhibit CTL4 activity, thereby inducing, eliciting, or enhancing immune responses. In an embodiment, the anti-CTLA-4 antibody may be ipilimumab (Bristol-Myers Squibb) or BN13 (BioXCell). In another embodiment, the anti-CTLA-4 antibody may be UC10-4F10-11, 9D9 or 9H10 (BioXCell) or a human or humanized counterpart thereof.

The amount of any specific therapeutic agent may depend on the type of the therapeutic agent (e.g. peptide antigen, small molecule drug, antibody, etc.). One skilled in the art can readily determine the amount of therapeutic agent needed in a particular application by empirical testing.

T-Helper Epitopes

In some embodiments, one or more T-helper epitopes may be used in the methods, dried preparations, compositions, uses or kits disclosed herein. In an embodiment, a T-helper epitope is used when at least one therapeutic agent is an antigen.

T-helper epitopes are a sequence of amino acids (natural or non-natural amino acids) that have T-helper activity. T-helper epitopes are recognised by T-helper lymphocytes, which play an important role in establishing and maximising the capabilities of the immune system, and are involved in activating and directing other immune cells, such as for example cytotoxic T lymphocytes. A T-helper epitope can consist of a continuous or discontinuous epitope. Hence not every amino acid of a T-helper is necessarily part of the epitope.

Accordingly, T-helper epitopes, including analogs and segments of T-helper epitopes, are capable of enhancing or stimulating an immune response. Immunodominant T-helper epitopes are broadly reactive in animal and human populations with widely divergent MHC types (Celis 1988, Demotz 1989, Chong 1992). The T-helper domain of the subject peptides may have from about 10 to about 50 amino acids, and more particularly about 10 to about 30 amino acids. When multiple T-helper epitopes are present, then each T-helper epitope acts independently.

In another embodiment, the T-helper epitope may be a T-helper epitope analog or a T-helper segment. T-helper epitope analogs may include substitutions, deletions and insertions of from one to about 10 amino acid residues in the T-helper epitope. T-helper segments are contiguous portions of a T-helper epitope that are sufficient to enhance or stimulate an immune response. An example of T-helper segments is a series of overlapping peptides that are derived from a single longer peptide.

In some embodiments, the T-helper epitope may form part of a peptide antigen described herein. In particular, if the peptide antigen is of sufficient size, it may contain an epitope that functions as a T-helper epitope. In other embodiments, the T-helper epitope is a separate molecule from the peptide antigen. In other embodiments, the T-helper epitope may be fused to the peptide antigen.

In a particular embodiment, the T-helper epitope may be the modified Tetanus toxin peptide A16L (amino acids 830 to 844; AQYIKANSKFIGITEL; SEQ ID NO: 1), with an alanine residue added to its amino terminus to enhance stability (Slingluff 2001).

Other sources of T-helper epitopes which may be used include, for example, hepatitis B surface antigen helper T cell epitopes, pertussis toxin helper T cell epitopes, measles virus F protein helper T cell epitope, Chlamydia trachomitis major outer membrane protein helper T cell epitope, diphtheria toxin helper T cell epitopes, Plasmodium falciparum circumsporozoite helper T cell epitopes, Schistosoma mansoni triose phosphate isomerase helper T cell epitopes, Escherichia coli TraT helper T cell epitopes and immune-enhancing analogs and segments of any of these T-helper epitopes.

In some embodiments, the T-helper epitope may be a universal T-helper epitope. A universal T-helper epitope as used herein refers to a peptide or other immunogenic molecule, or a fragment thereof, that binds to a multiplicity of MEW class II molecules in a manner that activates T cell function in a class II (CD4+ T cells)-restricted manner. An example of a universal T-helper epitope is PADRE (pan-DR epitope) comprising the peptide sequence AKXVAAWTLKAAA, wherein X may be cyclohexylalanyl (SEQ ID NO: 29). PADRE specifically has a CD4+T-helper epitope, that is, it stimulates induction of a PADRE-specific CD4+T-helper response.

In addition to the modified tetanus toxin peptide A16L mentioned earlier, Tetanus toxoid has other T-helper epitopes that work in the similar manner as PADRE. Tetanus and diphtheria toxins have universal epitopes for human CD4+ cells (Diethelm-Okita 2000). In another embodiment, the T-helper epitope may be a tetanus toxoid peptide such as F21E comprising the peptide sequence FNNFTVSFWLRVPKVSASHLE (amino acids 947 to 967; SEQ ID NO: 30).

Many other T-helper epitopes are known in the art, and any of these T-helper epitopes may be used in the practice of the methods, dried preparations, compositions, uses and kits disclosed herein.

In an embodiment, the dried preparations or compositions disclosed herein comprise a single type T-helper epitope. In another embodiment, the dried preparations or compositions disclosed herein comprise multiple different types of T-helper epitopes (e.g. 1, 2, 3, 4 or 5 different T-helper epitopes).

In an embodiment, the dried preparations or compositions disclosed herein do not comprise a T-helper epitope. For example, such may be the case when the therapeutic agent is not an antigen.

The amount of T-helper epitope used may depend on the type(s) and amount of therapeutic agent and on the type of T-helper epitope. One skilled in the art can readily determine the amount of T-helper epitope needed in a particular application by empirical testing.

Adjuvants

In some embodiments, one or more adjuvants may be used in the methods, dried preparations, compositions, uses or kits disclosed herein.

A large number of adjuvants have been described and are known to those skilled in the art. Exemplary adjuvants include, without limitation, alum, other compounds of aluminum, Bacillus of Calmette and Guerin (BCG), TiterMax™, Ribi™, Freund's Complete Adjuvant (FCA), CpG-containing oligodeoxynucleotides (CpG ODN), lipid A mimics or analogs thereof, lipopeptides and polyI:C polynucleotides.

In an embodiment, the adjuvant is a CpG ODN. CpG ODNs are DNA molecules that contain one or more unmethylated CpG motifs (consisting of a central unmethylated CG dinucleotide plus flanking regions). An exemplary CpG ODN is 5′-TCCATGACGTTCCTGACGTT-3′ (SEQ ID NO: 31). The skilled person can readily select other appropriate CpG ODNs on the basis of the target species and efficacy.

In an embodiment, the adjuvant may be a polyI:C polynucleotide.

PolyI:C polynucleotides are polynucleotide molecules (either RNA or DNA or a combination of DNA and RNA) containing inosinic acid residues (I) and cytidylic acid residues (C), and which induce the production of inflammatory cytokines, such as interferon. In an embodiment, the polyI:C polynucleotide is double-stranded. In such embodiments, they may be composed of one strand consisting entirely of cytosine-containing nucleotides and one strand consisting entirely of inosine-containing nucleotides, although other configurations are possible. For instance, each strand may contain both cytosine-containing and inosine-containing nucleotides. In some instances, either or both strands may additionally contain one or more non-cytosine or non-inosine nucleotides.

It has been reported that polyI:C can be segmented every 16 residues without an effect on its interferon activating potential (Bobst 1981). Furthermore, the interferon inducing potential of a polyI:C molecule mismatched by introducing a uridine residue every 12 repeating cytidylic acid residues (Hendrix 1993), suggests that a minimal double stranded polyI:C molecule of 12 residues is sufficient to promote interferon production. Others have also suggested that regions as small as 6-12 residues, which correspond to 0.5-1 helical turn of the double stranded polynucleotide, are capable of triggering the induction process (Greene 1978). If synthetically made, polyI:C polynucleotides are typically about 20 or more residues in length (commonly 22, 24, 26, 28 or 30 residues in length). If semi-synthetically made (e.g. using an enzyme), the length of the strand may be 500, 1000 or more residues.

Accordingly, as used herein, a “polyI:C”, “polyI:C polynucleotide” or “polyI:C polynucleotide adjuvant” is a double- or single-stranded polynucleotide molecule (RNA or DNA or a combination of DNA and RNA), each strand of which contains at least 6 contiguous inosinic or cytidylic acid residues, or 6 contiguous residues selected from inosinic acid and cytidylic acid in any order (e.g. IICIIC or ICICIC), and which is capable of inducing or enhancing the production of at least one inflammatory cytokine, such as interferon, in a mammalian subject. PolyI:C polynucleotides will typically have a length of about 8, 10, 12, 14, 16, 18, 20, 22, 24, 25, 26, 28, 30, 35, 40, 45, 50, 55, 60, 65, 70, 75, 80, 85, 90, 95, 100, 150, 200, 250, 300, 500, 1000 or more residues. Preferred polyI:C polynucleotides may have a minimum length of about 6, 8, 10, 12, 14, 16, 18, 20, 22, 24, 26, 28, or 30 nucleotides and a maximum length of about 1000, 500, 300, 200, 100, 90, 80, 70, 60, 50, 45 or 40 nucleotides.

Each strand of a double-stranded polyI:C polynucleotide may be a homopolymer of inosinic or cytidylic acid residues, or each strand may be a heteropolymer containing both inosinic and cytidylic acid residues. In either case, the polymer may be interrupted by one or more non-inosinic or non-cytidylic acid residues (e.g. uridine), provided there is at least one contiguous region of 6 I, 6 C or 6 I/C residues as described above. Typically, each strand of a polyI:C polynucleotide will contain no more than 1 non-I/C residue per 6 I/C residues, more preferably, no more than 1 non-I/C residue per every 8, 10, 12, 14, 16, 18, 20, 22, 24, 26, 28 or 30 I/C residues.

The inosinic acid or cytidylic acid (or other) residues in the polyI:C polynucleotide may be derivatized or modified as is known in the art, provided the ability of the polyI:C polynucleotide to promote the production of an inflammatory cytokine, such as interferon, is retained. Non-limiting examples of derivatives or modifications include e.g. azido modifications, fluoro modifications, or the use of thioester (or similar) linkages instead of natural phosphodiester linkages to enhance stability in vivo. The polyI:C polynucleotide may also be modified to e.g. enhance its resistance to degradation in vivo by e.g. complexing the molecule with positively charged poly-lysine and carboxymethylcellulose, or with a positively charged synthetic peptide.

In an embodiment, the polyI:C polynucleotide may be a single-stranded molecule containing inosinic acid residues (I) and cytidylic acid residues (C). As an example, and without limitation, the single-stranded polyI:C may be a sequence of repeating dIdC. In a particular embodiment, the sequence of the single-stranded polyI:C may be a 26-mer sequence of (IC)₁₃, i.e. ICICICICICICICICICICICICIC (SEQ ID NO: 32). As the skilled person will appreciate, due to their nature (e.g. complementarity), it is anticipated that these single-stranded molecules of repeating dIdC would naturally form homodimers, so they are conceptually similar to polyI/polyC dimers.

In an embodiment, the polyI:C polynucleotide adjuvant is a traditional form of polyI:C with an approximate molecular weight of 989,486 Daltons, containing a mixture of varying strand lengths of polyI and polyC of several hundred base pairs (Thermo Scientific; USA).

In an embodiment, the adjuvant may be one that activates or increases the activity of TLR2. As used herein, an adjuvant which “activates” or “increases the activity” of a TLR2 includes any adjuvant, in some embodiments a lipid-based adjuvant, which acts as a TLR2 agonist. Further, activating or increasing the activity of TLR2 encompasses its activation in any monomeric, homodimeric or heterodimeric form, and particularly includes the activation of TLR2 as a heterodimer with TLR1 or TLR6 (i.e. TLR1/2 or TLR2/6). Exemplary embodiments of an adjuvant that activates or increases the activity of TLR2 include lipid-based adjuvants, such as those described in WO2013/049941.

In an embodiment, the adjuvant may be a lipid-based adjuvant, such as disclosed for example in WO2013/049941. In an embodiment, the lipid-based adjuvant is one that comprises a palmitic acid moiety such as dipalmitoyl-S-glyceryl-cysteine (PAM2Cys) or tripalmitoyl-S-glyceryl-cysteine (PAM3Cys). In an embodiment, the adjuvant is a lipopeptide. Exemplary lipopeptides include, without limitation, PAM2Cys-Ser-(Lys)4 (SEQ ID NO: 33) or PAM3Cys-Ser-(Lys)4 (SEQ ID NO: 33).

In an embodiment, the adjuvant is PAM3Cys-SKKKK (EMC Microcollections, Germany; SEQ ID NO: 33) or a variant, homolog and analog thereof. The PAM2 family of lipopeptides has been shown to be an effective alternative to the PAM3 family of lipopeptides.

In an embodiment, the adjuvant may be a lipid A mimic or analog adjuvant, such as for example those disclosed in WO2016/109880 and the references cited therein. In a particular embodiment, the adjuvant may be JL-265 or JL-266 as disclosed in WO2016/109880.

In an embodiment, a combination of a polyI:C polynucleotide adjuvant and a lipid-based adjuvant may be used, such as described in the adjuvanting system disclosed in WO2017/083963.

Further examples of adjuvants that may be used include, without limitation, chemokines, colony stimulating factors, cytokines, 1018 ISS, aluminum salts, Amplivax, AS04, AS15, ABM2, Adjumer, Algammulin, AS01B, AS02 (SBASA), ASO2A, BCG, Calcitriol, Chitosan, Cholera toxin, CP-870,893, CpG, polyI:C, CyaA, DETOX (Ribi Immunochemicals), Dimethyldioctadecylammonium bromide (DDA), Dibutyl phthalate (DBP), dSLIM, Gamma inulin, GM-CSF, GMDP, Glycerol, IC30, IC31, Imiquimod, ImuFact IMP321, IS Patch, ISCOM, ISCOMATRIX, Juvlmmune, LipoVac, LPS, lipid core protein, MF59, monophosphoryl lipid A and analogs or mimics thereof, Montanide® IMS1312, Montanide® based adjuvants (e.g. Montanide ISA-51, -50 and -70), OK-432, OM-174, OM-197-MP-EC, ONTAK, PepTel vector system, other palmitoyl based molecules, PLG microparticles, resiquimod, squalene, SLR172, YF-17 DBCG, QS21, QuilA, P1005, Poloxamer, Saponin, synthetic polynucleotides, Zymosan, pertussis toxin.

In an embodiment, at least one of the therapeutic agents may be coupled to at least one of the adjuvants. In an embodiment, the adjuvant is not coupled to any of the therapeutic agents.

The amount of adjuvant used may depend on the type(s) and amount of therapeutic agent and on the type of adjuvant. One skilled in the art can readily determine the amount of adjuvant needed in a particular application by empirical testing.

Surfactants

In an embodiment, the compositions disclosed herein may comprise one or more surfactants. The surfactant may be a single agent or a mixture of agents. The surfactant(s) should be pharmaceutically and/or immunologically acceptable.

In some embodiments, a surfactant may be used to assist in stabilizing the lipid-based structures having a single layer lipid assembly, therapeutic agents and/or other components (e.g. adjuvant and/or T-helper epitope) in the hydrophobic carrier. The use of a surfactant may, for example, promote more even distribution of the mixture of these components by reducing surface tensions. In an embodiment, a surfactant may be used when the compositions disclosed herein are to contain multiple different therapeutic agents (e.g. five or more different peptide antigens) or a relatively high concentration of therapeutic agent (e.g. ≥5 mg/mg total of therapeutic agent).

The surfactant may be amphipathic and therefore, the surfactant may include a broad range of compounds. Examples of surfactants which may be used include polysorbates, which are oily liquids derived from polyethylene glycolyated sorbital, and sorbitan esters. Polysorbates may include, for example, sorbitan monooleate. Typical surfactants are well-known in the art and include, without limitation, mannide oleate (Arlacel™ A), lecithin, Tween™ 80, Spans™ 20, 80, 83 and 85. In an embodiment, the surfactant for use in the compositions is mannide oleate. In an embodiment, the surfactant for use in the compositions is Span80.

The surfactant is generally pre-mixed with the hydrophobic carrier. In some embodiments, a hydrophobic carrier which already contains a surfactant may be used. For example, a hydrophobic carrier such Montanide™ ISA 51 already contains the surfactant mannide oleate. In other embodiments, the hydrophobic carrier may be mixed with a surfactant before combining with the other components (e.g. the dried lipid/therapeutic agent preparation).

The surfactant is used in an amount effective to promote even distribution of the dried preparation in the hydrophobic carrier and/or to assist in the formation of the single layer assembly of the lipid-based structures. Typically, the volume ratio (v/v) of hydrophobic carrier to surfactant is in the range of about 4:1 to about 15:1.

In an embodiment, the compositions do not contain a surfactant. In such embodiments, the small uniform size of the sized lipid vesicle particles may permit the lipids to easily rearrange to form the lipid-based structures having a single layer lipid assembly in the presence of the therapeutic agents and/or other components (e.g. adjuvant and/or T-helper epitope) in the hydrophobic carrier. Thus, in such embodiments, a surfactant is not required.

### EMBODIMENTS

Particular embodiments of the invention include, without limitation, the following:

(1) A method for preparing a dried preparation comprising lipids and a therapeutic agent, said method comprising the steps of: (a) providing lipid vesicle particles having a mean particle size of ≤120 nm and a polydispersity index (PDI) of ≤0.1; (b) mixing the lipid vesicle particles with at least one solubilized therapeutic agent to form a mixture; and (c) drying the mixture formed in step (b) to form a dried preparation comprising lipids and a therapeutic agent.

(2) The method of paragraph (1), wherein step (a) comprises sizing lipid vesicle particles to provide the lipid vesicle particles having a mean particle size of ≤120 nm and a PDI of ≤0.1.

(3) The method of paragraph (2), wherein the sizing is by filter-extrusion.

(4) The method of paragraph (2) or (3), wherein the sizing is by extrusion through one or more polycarbonate membranes, such as a 0.2 μm polycarbonate membrane, a 0.1 μm polycarbonate membrane and/or a 0.08 μm polycarbonate membrane.

(5) The method of any one of paragraphs (2) to (4), wherein the sizing is by extrusion (i) 20-40 times through a 0.2 μm polycarbonate membrane, and then 10-20 times through a 0.1 μm polycarbonate membrane; or (ii) 20-40 times through a 0.2 polycarbonate membrane, then 10-20 times through a 0.1 μm polycarbonate membrane, and then 10-20 times through a 0.08 μm polycarbonate membrane.

(6) The method of any one of paragraphs (2) to (5), wherein the sizing is by extrusion (i) 25 times through a 0.2 μm polycarbonate membrane, and then 10 times through a 0.1 μm polycarbonate membrane or (ii) 25 times through a 0.2 μm polycarbonate membrane, then 10 times through a 0.1 μm polycarbonate membrane, and then 15 times through a 0.08 μm polycarbonate membrane.

(7) The method of any one of paragraphs (1) to (6), wherein the mean particle size of the lipid vesicle particles is between about 80 nm and about 120 nm.

(8) The method of any one of paragraphs (1) to (7), wherein the mean particle size of the lipid vesicle particles is about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm or about 115 nm.

(9) The method of any one of paragraphs (1) to (8), wherein the mean particle size of the lipid vesicle particles is ≤100 nm.

(10) The method of any one of paragraphs (1) to (9), wherein the lipid vesicle particles comprise a synthetic lipid.

(11) The method of paragraph (10), wherein the lipid vesicle particles comprise synthetic dioleoyl phosphatidylcholine (DOPC) or synthetic DOPC and cholesterol.

(12) The method of paragraph (11), wherein the lipid vesicle particles comprise synthetic DOPC and cholesterol at a DOPC:cholesterol ratio of 10:1 (w/w).

(13) The method of any one of paragraphs (1) to (12), wherein the lipid vesicle particles of step (a) are prepared from a liposome precursor.

(14) The method of paragraph (13), wherein the liposome precursor is Presome®.

(15) The method of any one of paragraphs (1) to (14), wherein the lipid vesicle particles are liposomes.

(16) The method of paragraph (15), wherein the liposomes are unilamellar, multilamellar, multivesicular, or a mixture thereof.

(17) The method of any one of paragraphs (1) to (16), wherein the at least one therapeutic agent is solubilized in one or more of sodium acetate, sodium phosphate or sodium hydroxide.

(18) The method of any one of paragraphs (1) to (17), wherein the at least one therapeutic agent is solubilized in one or more of 0.1 M sodium hydroxide, 100 mM sodium acetate having a pH of 6.0±1.0, 100 mM sodium acetate having a pH of 9.5±1.0, 50 mM sodium phosphate having a pH of 7.0±1.0 or 100 mM sodium phosphate having a pH of 6.0±1.0.

(19) The method of any one of paragraphs (1) to (18), wherein the mixing of step (b) is performed in a sodium acetate or sodium phosphate solution.

(20) The method of paragraph (19), wherein the mixing of step (b) is performed in 25-250 mM sodium acetate having a pH in the range of 6.0-10.5 or 25-250 mM sodium phosphate having a pH in the range of 6.0-8.0.

(21) The method of paragraph (19), wherein the mixing of step (b) is performed in 50 mM sodium acetate having a pH of 6.0±1.0, 100 mM sodium acetate having a pH of 9.5±1.0, 50 mM sodium phosphate having a pH of 7.0±1.0 or 100 mM sodium phosphate having a pH of 6.0±1.0.

(22) The method of paragraph (19), wherein the mixing of step (b) is performed in 50 mM sodium phosphate having a pH of 7.0, 100 mM sodium phosphate having a pH of 6.0, 50 mM sodium acetate having a pH of 6.0, or 100 mM sodium acetate having a pH of 9.5.

(23) The method of any one of paragraphs (1) to (22), wherein the therapeutic agent is a peptide antigen, a DNA or RNA polynucleotide that encodes a polypeptide, a hormone, a cytokine, an allergen, a catalytic DNA (deoxyribozyme), a catalytic RNA (ribozyme), an antisense RNA, an interfering RNA, an antagomir, a small molecule drug, a biologic drug, an antibody, or a fragment or derivative of any one thereof; or a mixture thereof.

(24) The method of any one of paragraphs (1) to (22), wherein the therapeutic agent is one or more peptide antigens.

(25) The method of paragraph (24), wherein the one or more peptide antigens are 20-30 amino acids in length.

(26) The method of paragraph (24) or (25), wherein the one or more peptide antigens are neoantigens.

(27) The method of paragraph (24), wherein the one or more peptide antigens are derived from human papillomavirus (HPV), human immunodeficiency virus (HIV), respiratory syncytial virus (RSV), Bacillus anthracis, Plasmodium, or a survivin polypeptide.

(28) The method of paragraph (27), wherein the one or more peptide antigens are FTELTLGEF (SEQ ID NO: 4), LMLGEFLKL (SEQ ID NO: 5), RISTFKNWPK (SEQ ID NO: 6), STFKNWPFL (SEQ ID NO: 7) or LPPAWQPFL (SEQ ID NO: 8); or any combination thereof.

(29) The method of paragraph (27), wherein the one or more peptide antigens are NKLCEYNVFHNKTFELPRARVNT (SEQ ID NO: 2) and/or NKLSEHKTFCNKTLEQGQMYQINT (SEQ ID NO: 3).

(30) The method of any one of paragraphs (24) to (27), wherein step (b) comprises mixing five or more different solubilized peptide antigens with the lipid vesicle particles.

(31) The method of paragraph (30), wherein step (b) comprises mixing up to 30 different solubilized peptide antigens with the lipid vesicle particles.

(32) The method of paragraph (30), wherein step (b) comprises mixing 10, 11, 12, 13, 14, 15, 16, 17, 18, 19 or 20 different solubilized peptide antigens with the lipid vesicle particles.

(33) The method of any one of paragraphs (30) to (32), wherein, after step (b), each of the different solubilized peptide antigens is at a concentration of at least about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml or 1.0 mg/ml.

(34) The method of any one of paragraphs (30) to (33), wherein, after step (b), each of the different solubilized peptide antigens is at a concentration of about 0.5 mg/ml.

(35) The method of any one of paragraphs (30) to (34), wherein the different solubilized peptide antigens are not pre-selected based on any characteristic relating to isoelectric point, solubility, stability and/or immunogenicity.

(36) The method of any one of paragraphs (30) to (35), wherein the different solubilized peptide antigens have one or more different characteristics relating to isoelectric point, solubility, stability and/or immunogenicity.

(37) The method of any one of paragraphs (30) to (36), wherein the different solubilized peptide antigens have a different length, sequence, molecular weight, charge, polarity, hydrophobicity and/or hydrophilicity.

(38) The method of any one of paragraphs (24) to (37), wherein step (b) further comprises mixing, in any order, a solubilized T-helper epitope with the lipid vesicle particles and the one or more peptide antigens.

(39) The method of paragraph (37), wherein step (b) comprises mixing 10-15 neoantigens with one solubilized T-helper epitope, wherein the T-helper epitope comprises the amino acid sequence AQYIKANSKFIGITEL (SEQ ID NO: 1).

(40) The method of any one of paragraphs (24) to (39), wherein step (b) further comprises mixing, in any order, an adjuvant with the lipid vesicle particles and the one or more peptide antigens.

(41) The method of paragraph (38) or (39), wherein step (b) comprises: (b1) providing an antigen stock comprising the one or more peptide antigens and the solubilized T-helper epitope; and (b2) mixing the antigen stock with the lipid vesicle particles to form the mixture.

(42) The method of paragraph (41), wherein, in step (b1), the antigen stock is prepared in 100 mM sodium hydroxide with each solubilized antigen having a concentration of about 2.0 mg/ml.

(43) The method of paragraph (42), wherein the antigen stock is diluted 1:1 with 50 mM sodium acetate having a pH of 6.0±0.5 to provide each solubilized antigen at a concentration of about 1.0 mg/ml.

(44) The method of any one of paragraphs (41) to (43), wherein after the mixing in step (b2) and prior to drying, the pH of the mixture is adjusted to 10±1.0.

(45) The method of any one of paragraphs (41) to (44), wherein step (b2) further comprises mixing the mixture with an adjuvant.

(46) The method of paragraph (40) or (45), wherein the adjuvant is a polyI:C nucleotide adjuvant.

(47) The method of any one of paragraphs (1) to (46) further comprising a step of sterile filtration of the mixture formed in step (b) prior to drying.

(48) The method of any one of paragraphs (1) to (47) further comprising, between steps (b) and (c), a step of confirming that the lipid vesicle particles still have a mean particle size of ≤120 nm and a polydispersity index (PDI) of ≤0.1.

(49) The method of any one of paragraphs (1) to (48), wherein the drying is performed by lyophilization, spray freeze-drying, or spray drying.

(50) The method of paragraph (49), wherein the drying is performed by lyophilization.

(51) The method of paragraph (50), wherein the lyophilization is performed by loading one or more containers comprising the mixture of step (b) into a bag, sealing the bag to form a sealed unit, and lyophilizing the sealed unit in a freeze-dryer.

(52) The method of paragraph (51), wherein the bag is a sterile, autoclaved bag.

(53) The method of paragraph (51) or (52), wherein the freeze-dryer is a benchtop freeze dryer.

(54) The method of any one of paragraphs (51) to (53), wherein the freeze-dryer contains more than one sealed unit during the lyophilization.

(55) The method of paragraph (54), wherein each sealed unit contains a different mixture prepared by steps (a) and (b).

(56) The method of any one of paragraphs (1) to (55), further comprising a step of evaluating the stability of the at least one solubilized therapeutic agent before and/or after the drying of step (c).

(57) The method of paragraph (56), wherein the stability of the therapeutic agents is evaluated by HPLC analysis.

(58) The method of paragraph (56) or (57), wherein the therapeutic agents are peptide antigens and at least 80% of the original peptide concentration of each peptide antigen is retained in undegraded form when evaluated before drying.

(59) The method of paragraph (60), wherein at least 75% of the original peptide concentration of each peptide antigen is retained in undegraded form when evaluated immediately after drying.

(60) The method of paragraph (58) or (59), wherein at least 70% of the original peptide concentration of each peptide antigen is retained in undegraded form when evaluated three months after drying.

(61) The method of any one of paragraphs (58) to (60), wherein one or more of the peptide antigens shows no degradation for up to 3 months after drying.

(62) A method for preparing a pharmaceutical composition comprising solubilizing the dried preparation obtained by the method of any one of paragraphs (1) to (61) in a hydrophobic carrier.

(63) The method of paragraph (62), wherein the hydrophobic carrier is mineral oil or a mannide oleate in mineral oil solution.

(64) The method of paragraph (62) or (63), wherein the hydrophobic carrier is Montanide® ISA 51.

(65) A pharmaceutical composition prepared by the method of any one of paragraphs (62) to (64).

(66) The pharmaceutical composition of paragraph (65), wherein the lipids are in the form of one or more lipid-based structures having a single layer lipid assembly in the hydrophobic carrier.

(67) The pharmaceutical composition of paragraph (66), wherein, in the hydrophobic carrier, the lipids are in the form of reverse micelles and/or aggregates of lipids with the hydrophobic part of the lipids oriented outwards toward the hydrophobic carrier and the hydrophilic part of the lipids aggregating as a core.

(68) A stable, water-free pharmaceutical composition comprising one or more lipid-based structures having a single layer lipid assembly, at least one therapeutic agent, and a hydrophobic carrier.

(69) The pharmaceutical composition of paragraph (68), wherein the therapeutic agent is a peptide antigen, a DNA or RNA polynucleotide that encodes a polypeptide, a hormone, a cytokine, an allergen, a catalytic DNA (deoxyribozyme), a catalytic RNA (ribozyme), an antisense RNA, an interfering RNA, an antagomir, a small molecule drug, a biologic drug, an antibody, or a fragment or derivative of any one thereof; or a mixture thereof.

(70) The pharmaceutical composition of paragraph (68) or (69), wherein the therapeutic agent is one or more peptide antigens.

(71) The pharmaceutical composition of paragraph (70), wherein the one or more peptide antigens are 20-30 amino acids in length.

(72) The pharmaceutical composition of paragraph (70) or (71), wherein the one or more peptide antigens are neoantigens.

(73) The pharmaceutical composition of paragraph (70), wherein the one or more peptide antigens are derived from human papillomavirus (HPV), human immunodeficiency virus (HIV), respiratory syncytial virus (RSV), Bacillus anthracis, Plasmodium, or a survivin polypeptide.

(74) The pharmaceutical composition of paragraph (73), wherein the one or more peptide antigens are FTELTLGEF (SEQ ID NO: 4), LMLGEFLKL (SEQ ID NO: 5), RISTFKNWPK (SEQ ID NO: 6), STFKNWPFL (SEQ ID NO: 7) or LPPAWQPFL (SEQ ID NO: 8); or any combination thereof.

(75) The pharmaceutical composition of paragraph (73), wherein the one or more peptide antigens are NKLCEYNVFHNKTFELPRARVNT (SEQ ID NO: 2) and/or NKLSEHKTFCNKTLEQGQMYQINT (SEQ ID NO: 3).

(76) The pharmaceutical composition of any one of paragraphs (70) to (73), which comprises five or more different peptide antigens.

(77) The pharmaceutical composition of paragraph (76), which comprises up to 30 different peptide antigens.

(78) The pharmaceutical composition of paragraph (76), which comprises 10, 11, 12, 13, 14, 15, 16, 17, 18, 19 or 20 different peptide antigens.

(79) The pharmaceutical composition of any one of paragraphs (76) to (78), wherein each of the peptide antigens is, independently, at a concentration of between about 0.1 μg/μl and about 5.0 μg/μl.

(80) The pharmaceutical composition of any one of paragraphs (76) to (79), wherein each of the peptide antigens is, independently, at a concentration of about 0.25 μg/μl, about 0.5 μg/μl, about 0.75 μg/μl, about 1.0 μg/μl, about 1.25 μg/μl, about 1.5 μg/μl, about 1.75 μg/μl, about 2.0 μg/μl, about 2.25 μg/μl or about 2.5 μg/μl.

(81) The pharmaceutical composition of any one of paragraphs (76) to (80), which comprises 10 or more different peptide antigens, each at a concentration of at least about 0.5 μg/μl.

(82) The pharmaceutical composition of any one of paragraphs (76) to (81), wherein the peptide antigens are not pre-selected based on any characteristic relating to isoelectric point, solubility, stability and/or immunogenicity.

(83) The pharmaceutical composition of any one of paragraphs (76) to (82), wherein the peptide antigens have one or more different characteristics relating to isoelectric point, solubility, stability and/or immunogenicity.

(84) The pharmaceutical composition of any one of paragraphs (76) to (83), wherein the peptide antigens have a different length, sequence, molecular weight, charge, polarity, hydrophobicity and/or hydrophilicity.

(85) The pharmaceutical composition of any one of paragraphs (68) to (84), further comprising one or both of a T-helper epitope and an adjuvant.

(86) The pharmaceutical composition of any one of paragraphs (68) to (85), wherein the hydrophobic carrier is mineral oil or a mannide oleate in mineral oil solution.

(87) The pharmaceutical composition of any one of paragraphs (68) to (86), wherein the hydrophobic carrier is Montanide® ISA 51.

(88) The pharmaceutical composition of any one of paragraphs (68) to (87), wherein the one or more lipid-based structures having a single layer lipid assembly comprise aggregates of lipids with the hydrophobic part of the lipids oriented outwards toward the hydrophobic carrier and the hydrophilic part of the lipids aggregating as a core.

(89) The pharmaceutical composition of any one of paragraphs (68) to (88), wherein the one or more lipid-based structures having a single layer lipid assembly comprise reverse micelles.

(90) The pharmaceutical composition of any one of paragraphs (68) to (89), wherein one or more of the at least one therapeutic agent are inside the lipid-based structures.

(91) The pharmaceutical composition of any one of paragraphs (68) to (90), wherein one or more of the at least one therapeutic agent are outside the lipid-based structures.

(92) The pharmaceutical composition of any one of paragraphs (68) to (91), which is a clear solution.

(93) The pharmaceutical composition of any one of paragraphs (68) to (92), which has no visible precipitate.

(94) A stable, water-free pharmaceutical composition comprising one or more lipid-based structures having a single layer assembly, five or more different peptide neoantigens, and a hydrophobic carrier, wherein the peptide neoantigens are not pre-selected based on any characteristic relating to isoelectric point, solubility, stability and/or immunogenicity.

(95) The pharmaceutical composition of paragraph (94), which comprises up to 30 different peptide neoantigens.

(96) The pharmaceutical composition of paragraph (94) or (95), which comprises 10, 11, 12, 13, 14, 15, 16, 17, 18, 19 or 20 different peptide neoantigens.

(97) The pharmaceutical composition of any one of paragraphs (94) to (96), wherein each of the peptide neoantigens is, independently, at a concentration of between about 0.1 μg/μl and about 5.0 μg/μl.

(98) The pharmaceutical composition of any one of paragraphs (94) to (97), wherein each of the peptide neoantigens is, independently, at a concentration of about 0.25 μg/μl, about 0.5 μg/μl, about 0.75 μg/μl, about 1.0 μg/μl, about 1.25 μg/μl, about 1.5 μg/μl, about 1.75 μg/μl, about 2.0 μg/μl, about 2.25 μg/μl or about 2.5 μg/μl.

(99) The pharmaceutical composition of any one of paragraphs (94) to (98), which comprises at least 14 different peptide neoantigens, each at a concentration of at least about 0.5 μg/μl.

(100) The pharmaceutical composition of any one of paragraphs (94) to (99), wherein the peptide neoantigens are 20-30 amino acids in length.

(101) The pharmaceutical composition of any one of paragraphs (94) to (100), wherein the peptide neoantigens have one or more different characteristics relating to isoelectric point, solubility, stability and/or immunogenicity.

(102) The pharmaceutical composition of any one of paragraphs (94) to (101), wherein the neoantigens have a different length, sequence, molecular weight, charge, polarity, hydrophobicity and/or hydrophilicity.

(103) The pharmaceutical composition of any one of paragraphs (94) to (102), further comprising one or both of a T-helper epitope and an adjuvant.

(104) The pharmaceutical composition of any one of paragraphs (94) to (103), wherein the hydrophobic carrier is mineral oil or a mannide oleate in mineral oil solution.

(105) The pharmaceutical composition of any one of paragraphs (94) to (104), wherein the hydrophobic carrier is Montanide® ISA 51.

(106) The pharmaceutical composition of any one of paragraphs (94) to (105), wherein the lipid-based structures are vesicular structures with the hydrophobic part of the lipids oriented outwards toward the hydrophobic carrier.

(107) The pharmaceutical composition of any one of paragraphs (94) to (106), wherein the one or more lipid-based structures having a single layer lipid assembly comprise reverse micelles and/or aggregates of lipids with the hydrophobic part of the lipids oriented outwards toward the hydrophobic carrier and the hydrophilic part of the lipids aggregating as a core.

(108) The pharmaceutical composition of any one of paragraphs (94) to (107), wherein one or more of the at least one therapeutic agent are inside the lipid-based structures.

(109) The pharmaceutical composition of any one of paragraphs (94) to (108), wherein one or more of the at least one therapeutic agent are outside the lipid-based structures.

(110) The pharmaceutical composition of any one of paragraphs (94) to (109), which is a clear solution.

(111) The pharmaceutical composition of any one of paragraphs (94) to (110), which has no visible precipitate.

(112) The pharmaceutical composition of any one of paragraphs (65) to (111), which is capable of being formulated as a microdose for administration by injection.

(113) The pharmaceutical composition of paragraph (112), wherein the microdose is a single dose volume of about 50 μl, 55 μl, 60 μl, 65 μl, 70 μl or 75 μl.

(114) The pharmaceutical composition of any one of paragraphs (66) to (112), wherein the size of the lipid-based structures is between about 2 nm to about 10 nm in diameter.

(115) A method of inducing an antibody and/or CTL immune response in a subject comprising administering to the subject the pharmaceutical composition of any one of paragraphs (65) to (114).

(116) The method of paragraph (115), which is for treating cancer or an infectious disease.

(117) Use of the pharmaceutical composition of any one of paragraphs (65) to (114) for inducing an antibody and/or CTL immune response in a subject.

(118) The use of paragraph (117), which is for the treatment of cancer.

(119) The use of paragraph (117), which is for the treatment of an infectious disease.

(120) A kit for preparing a pharmaceutical composition for inducing an antibody and/or CTL immune response, the kit comprising: a container comprising a dried preparation prepared by the method of any one of paragraphs (1) to (62); and a container comprising a hydrophobic carrier.

(121) The kit of paragraph (120), wherein the dried preparation comprises ten or more different peptide antigens.

(122) The kit of paragraph (120) or (121), wherein the hydrophobic carrier is mineral oil or a mannide oleate in mineral oil solution.

The invention is further illustrated by the following non-limiting examples.

### EXAMPLES

The invention will now be described by way of non-limiting examples having regard to the appended drawings.

### Example 1

Dried Lipid/Therapeutic Agent Preparation

To prepare a dried lipid/therapeutic agent preparation, a 10:1 (w:w) homogenous mixture of DOPC and cholesterol (Lipoid GmbH, Germany) was added to sodium phosphate, 100 mM, pH 7.0 at a concentration of 132 mg/ml, with shaking at 300 RPM for about 1 hour to form lipid vesicle particles. The mixture was then sized by passing the material 25 times through a 0.2 μm polycarbonate membrane and 10 times through a 0.1 μm polycarbonate membrane to attain a mean particle size of ≤120 nm with a pdi of ≤0.1. To the sized lipid vesicle particles, an HPV antigen (Y9T peptide; YMLNLGPET; SEQ ID NO: 10) was added at a concentration of 0.5 mg/mL and an adjuvant (dIdC oligonucleotide) was added at a concentration of 0.2 mg/mL, both with shaking at 300 RPM for about 15 minutes. A T-helper peptide (A16L peptide; AQYIKANSKFIGITEL; SEQ ID NO: 1) was then added at a concentration of 0.25 mg/ml to the sized lipid vesicle particle/antigen-adjuvant mixture with shaking at 300 RPM for about 5 minutes. The mixture was then subjected to serial sterile filtration by passing the mixture through Millipak-20 PVDF 0.22 μm membranes. The sterile solution was aliquoted into vials and freeze-dried to prepare the dried lipid/therapeutic agent preparation. This dried lipid/therapeutic agent preparation is hereinafter referred to as Batch #1.

To prepare a dried lipid/therapeutic agent preparation using a different buffer, the same protocol as above was followed, with the exception that sodium phosphate, 100 mM, pH 7.0 was replaced with sodium phosphate, 50 mM, pH 7.0. Two batches were prepared, which are hereinafter referred to as Batch #2 and Batch #3.

Solubilization in a Hydrophobic Carrier

The dried lipid/therapeutic agent preparations of Batches #1, #2 and #3 were each separately solubilized in an oil diluent (i.e. Montanide® ISA 51) to provide final compositions with the profile shown in the table below:

TABLE 1
Exemplary Product Profile

| | Concentration in Final Composition |
|---|---|
| Component | (mg/ml) |
| Antigen(s), e.g. protein/peptide | 1.00 |
| Adjuvant | 0.40 |
| T-helper peptide | 0.50 |
| DOPC/cholesterol (10:1 w/w) | 132.00 |

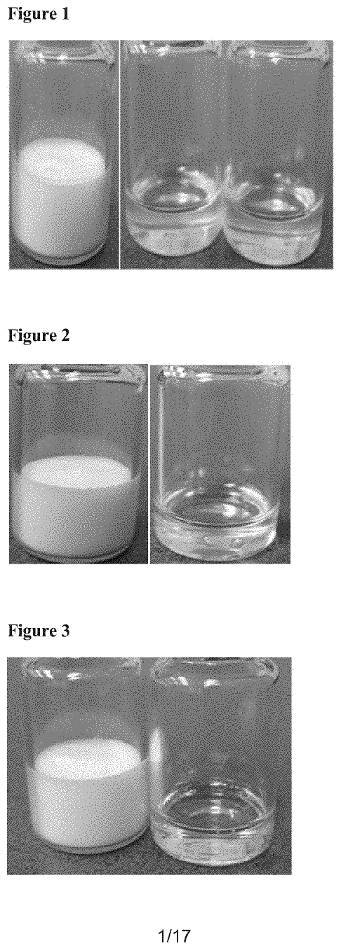

The characteristics of the resultant compositions after solubilization are described in the table below and in FIGS. 1-3.

TABLE 2
Product Characteristics—Effects of Buffer and pH

| | | Particle Size & | |
|---|---|---|---|
| | | pH Analysis | |
| Protocol & | Formulation | Before | Product |
| Batch Size | Method | Lyophilization | Description |
| Batch #1 | Sodium phosphate, | 109.7 nm | Clear solution |
| 160 vials | 100 mM, pH 7.0 | 0.087 pdi | with slight |
| | | pH 7.05 | precipitate |
| | | | [Figure 1] |
| Batch #2 | Sodium phosphate, | 108.5 nm | Clear solution |
| 190 vials | 50 mM, pH 7.0 | 0.083 pdi | [Figure 2] |
| | | pH 6.92 | |
| Batch #3 | Sodium phosphate, | 114.1 nm | Clear solution |
| 190 vials | 50 mM, pH 7.0 | 0.087 pdi | [Figure 3] |
| | | pH 6.96 | |

It was unexpectedly found that even when the peptide antigens are added outside pre-formed sized lipid vesicle particles, it was possible to solubilize the antigens in a hydrophobic carrier to obtain a clear solution without significant precipitate.

### Example 2

The effect of using sized lipid vesicle particles was compared to non-sized lipid vesicle particles.

To formulate compositions using sized lipid vesicle particles, the procedure in Example 1 for preparing Batch #1 was followed, with the exception that the following antigens were used:
Batch #4a was prepared using anthrax recombinant Protective Antigen purchased from List Biologics (Campbell, Calif.). This recombinant protein has an approximate molecular weight of 83,000 daltons and corresponds to the protective antigen, a cell binding component of the three-protein exotoxin produced by Bacillus anthracis. A concentration of 0.25 mg/ml was used in the formulation. This antigen is herein designated rPA. In this formulation, the dIdC oligonucleotide adjuvant used in Batch #1 was replaced with a lipopeptide adjuvant (R-Pam3Cys).
Batch #4b was prepared using HIV peptide antigens AMQ9 (AMQMLKETI; SEQ ID NO: 12) and RGP10 (RGPGRAFVTI; SEQ ID NO: 11) purchased from Genscript (Piscataway, N.J.). RGP10 peptide is derived from the V3 loop of HIV-1 gp120 and AMQ9 peptide is the immunodominant MHC class I epitope of gag for mice of the H-2Kd haplotype. Both are known to induce specific immune responses. Both were used at a concentration of 0.5 mg/mL in the formulation.
Batch #4c was prepared using five synthetic survivin peptide antigens purchased from Polypeptide (San Diego, Calif.) having the amino acid sequence: FTELTLGEF (SEQ ID NO: 4), LMLGEFLKL (SEQ ID NO: 5), RISTFKNWPK (SEQ ID NO: 6), STFKNWPFL (SEQ ID NO: 7) and LPPAWQPFL (SEQ ID NO: 8). In this formulation, the same T-helper epitope and adjuvant was used as compared to Batch #1. Each of the survivin peptide antigens was used at a concentration of 0.5 mg/ml in the formulation during manufacturing.
Batch #4d was prepared using an RSV peptide antigen (SHe A peptide; NKLCEYNVFHNKTFELPRARVNT; SEQ ID NO: 2) in dimer form. A concentration of 0.25 mg/ml was used in the formulation. In this formulation, the T-helper epitope was not included, and the dIdC oligonucleotide adjuvant was replaced with a lipopeptide adjuvant (R-Pam3Cys).

To formulate compositions using non-sized vesicle particles, the procedure in Example 1 for preparing Batch #1 was followed, with the exception that the step of sizing the material to attain a particle size of ≤120 nm with a pdi of ≤0.1 was not performed. Four batches were prepared, each using a different antigen as described above for Batches #4a, 4b, 4c and 4d. Specifically, the non-sized lipid vesicle particle compositions were Batch #5a (rPA antigen); Batch #5b (HIV antigen); Batch #5c (survivin antigen); and Batch #5d (RSV antigen). The quantity of antigen used was the same as for the sized lipid vesicle particle compositions.

The characteristics of the resultant compositions after solubilization in Montanide® ISA 51 are described in the table below and in FIG. 4.

TABLE 3
Product Characteristics—Effects of sized versus non-sized

| | Sized Lipid Vesicle Particle | Non-sized Lipid Vesicle Particle |
|---|---|---|
| Antigen | Compositions | Compositions |
| rPA | Batch 4a—Dried preparation | Batch 5a—Dried preparation |
| | looks good. Upon | looks good. Upon |
| | solubilization, formed | solubilization, formed slight |
| | clear solution. | hazy solution. |
| | [Figure 4, panel A] | [Figure 4, panel E] |
| HIV | Batch 4b—Dried preparation | Batch 5b—Dried preparation |
| | looks good. Upon | looks good. Upon |
| | solubilization, formed | solubilization, formed hazy |
| | clear solution. | solution. |
| | [Figure 4, panel B] | [Figure 4, panel F] |
| Survivin | Batch 4c—Dried preparation | Batch 5c—Dried preparation |
| | looks good. Upon | looks good. Upon |
| | solubilization, formed | solubilization, formed hazy |
| | clear solution. | solution. |
| | [Figure 4, panel C] | [Figure 4, panel G] |
| RSV | Batch 4d—Dried preparation | Batch 5d—Dried preparation |
| | looks good. Upon | looks good. Upon |
| | solubilization, formed | solubilization, formed hazy |
| | clear solution. | solution with slight precipitate. |
| | Sized lipid vesicle mean | Non-sized lipid vesicle mean |
| | particle size during | particle size during |
| | manufacture was | manufacture was |
| | 83.96 nm, pdi 0.092. | 2959 nm, pdi 0.362. |
| | [Figure 4, panel D] | [Figure 4, panel H] |

The results demonstrate that compositions prepared using sized lipid vesicle particles have improved pharmaceutical properties as compared to those prepared with non-sized lipid vesicle particles.

### Example 3

The effect of preparing sized lipid vesicle particles with and without cholesterol was studied.

Sized DOPC/Cholesterol Formulations

Batch #6: To prepare a dried survivin antigen preparation with DOPC/cholesterol lipid vesicle particles, a 10:1 (w:w) homogenous mixture of DOPC and cholesterol (Lipoid GmbH, Germany) was added to sodium acetate, 100 mM, pH 9.5 with shaking at 300 RPM for about 1 hour. The mixture was then sized by passing the material 25 times through a 0.2 μm polycarbonate membrane and then 10 times through a 0.1 μm polycarbonate membrane to attain a particle size of ≤120 nm with a pdi of ≤0.1. Five synthetic survivin peptide antigens (FTELTLGEF (SEQ ID NO: 4), LMLGEFLKL (SEQ ID NO: 5), RISTFKNWPK (SEQ ID NO: 6), STFKNWPFL (SEQ ID NO: 7) and LPPAWQPFL (SEQ ID NO: 8); each at 0.5 mg/mL) and a dIdC oligonucleotide adjuvant (0.2 mg/mL) were added with shaking at 300 RPM for about 15 minutes. A T-helper peptide (A16L peptide; AQYIKANSKFIGITEL (SEQ ID NO: 1); at 0.25 mg/mL) was then added to the sized lipid vesicle particle/survivin antigen-adjuvant mixture with shaking at 300 RPM for about 5 minutes. The mixture was then subjected to serial sterile filtration by passing the mixture through Millipak-20 PVDF 0.22 μm membranes. The sterile solution was aliquoted into vials and freeze-dried to prepare the dried lipid/therapeutic agent preparation.

Batch #7: To prepare a dried RSV antigen preparation with DOPC/cholesterol lipid vesicle particles, a 10:1 (w:w) homogenous mixture of DOPC and cholesterol (Lipoid GmbH, Germany) was added to sodium phosphate, 100 mM, pH 6.0 with shaking at 300 RPM for about 1 hour. The mixture was then sized by passing the material 25 times through a 0.2 μm polycarbonate membrane and then 10 times through a 0.1 μm polycarbonate membrane to attain a particle size of ≤120 nm with a pdi of ≤0.1. An RSV peptide antigen (SHe A peptide; NKLCEYNVFHNKTFELPRARVNT (SEQ ID NO: 2) at 0.25 mg/mL) in dimer form and a lipopeptide adjuvant (R-Pam3Cys; at 0.02 mg/mL) were added with shaking at 300 RPM for about 15 minutes. The mixture was then subjected to sterile filtration by passing the mixture through a single Millipak-20 PVDF 0.22 μm membrane. The sterile solution was aliquoted into vials and freeze-dried to prepare the dried lipid/therapeutic agent preparation.

Sized DOPC Formulations

Batch #8: To prepare a dried survivin antigen preparation with only DOPC lipid vesicle particles, DOPC (Lipoid GmbH, Germany) was added to sodium acetate, 100 mM, pH 9.5 with shaking at 300 RPM for about 1 hour. The mixture was then sized by passing the material 25 times through a 0.2 μm polycarbonate membrane, then 10 times through a 0.1 μm polycarbonate membrane, and then 15 times through a 0.08 μm polycarbonate membrane to attain a particle size of ≤100 nm with a pdi of ≤0.1. Five synthetic survivin peptide antigens (FTELTLGEF (SEQ ID NO: 4), LMLGEFLKL (SEQ ID NO: 5), RISTFKNWPK (SEQ ID NO: 6), STFKNWPFL (SEQ ID NO: 7) and LPPAWQPFL (SEQ ID NO: 8); each at 0.5 mg/mL) and a dIdC oligonucleotide adjuvant (0.2 mg/mL) were added with shaking at 300 RPM for about 15 minutes. A T-helper peptide (A16L peptide; AQYIKANSKFIGITEL (SEQ ID NO: 1); at 0.25 mg/mL) was then added to the sized lipid vesicle particle/survivin antigen-adjuvant mixture with shaking at 300 RPM for about 5 minutes. The mixture was then subjected to serial sterile filtration by passing the mixture through Millipak-20 PVDF 0.22 μm membranes. The sterile solution was aliquoted into vials and freeze-dried to prepare the dried lipid/therapeutic agent preparation.

Batch #9: To prepare a dried RSV antigen preparation with only DOPC lipid vesicle particles, DOPC (Lipoid GmbH, Germany) was added to sodium phosphate, 100 mM, pH 6.0 with shaking at 300 RPM for about 1 hour. The mixture was then sized by passing the material 25 times through a 0.2 μm polycarbonate membrane, then 10 times through a 0.1 μm polycarbonate membrane, and then 15 times through a 0.08 μm polycarbonate membrane to attain a particle size of ≤100 nm with a pdi of ≤0.1. An RSV peptide antigen (SHe A peptide; NKLCEYNVFHNKTFELPRARVNT (SEQ ID NO: 2) at 0.25 mg/mL) and a lipopeptide adjuvant (R-Pam3Cys; at 0.02 mg/mL) were added with shaking at 300 RPM for about 15 minutes. The mixture was then subjected to sterile filtration by passing the mixture through a single Millipak-20 PVDF 0.22 μm membrane. The sterile solution was aliquoted into vials and freeze-dried to prepare the dried lipid/therapeutic agent preparation.

The dried lipid/therapeutic agent preparations of Batches #6, #7, #8 and #9 were each separately solubilized in Montanide® ISA 51 to provide the final compositions.

The characteristics of the resultant compositions after solubilization are described in the table below and in FIG. 5.

TABLE 4
Product Characteristics—DOPC/cholesterol versus DOPC alone

| Parameters | DOPC + Cholesterol | DOPC Alone |
|---|---|---|
| Particle size for | 111.1 nm, 0.038 pdi | 97.07 nm, 0.086 pdi |
| survivin antigen | | |
| Particle size for | 115.10 nm, 0.092 pdi | 97.94 nm, 0.098 pdi |
| RSV antigen | | |
| Freeze-drying and | Dried preparation | Dried preparation |
| solubilization | looks good. Upon | looks good. Upon |
| details for survivin | solubilization, formed | solubilization, formed |
| antigen | clear solution. | clear solution. |
| | [Figure 5, panel A] | [Figure 5, panel C] |
| Freeze-drying and | Dried preparation | Dried preparation |
| solubilization | looks good. Upon | looks good. Upon |
| details for RSV | solubilization, formed | solubilization, formed |
| antigen | clear solution. | clear solution. |
| | [Figure 5, panel B] | [Figure 5, panel D] |
| Stability | ≥5 years | Ongoing |

The results demonstrate that compositions prepared using both DOPC and cholesterol or DOPC alone have similar pharmaceutical properties in respect of solubilization in an oil carrier.

### Example 4

This method disclosed herein have exemplary application in formulating peptide neoantigens.

The neoantigens used in this example are shown in the table below. These antigens were identified and published by Castle et al. (2012) from the common mouse melanoma tumor, B16-F10.

TABLE 5
List of neoantigen peptides

| Peptide | Sequence |
|---|---|
| Mut17 | VVDRNPQFLDPVLAYLMKGLCEKPLAS |
| | (SEQ ID NO: 34) |
| Mut20 | FRRKAFLHWYTGEAMDEMEFTEAESNM |
| | (SEQ ID NO: 35) |
| Mut22 | PKPDFSQLQRNILPSNPRVTRFHINWD |
| | (SEQ ID NO: 36) |
| Mut24 | TAVITPPTTTTKKARVSTPKPATPSTD |
| | (SEQ ID NO: 37) |
| Mut25 | STANYNTSHLNNDVWQIFENPVDWKEK |
| | (SEQ ID NO: 26) |
| Mut28 | NIEGIDKLTQLKKPFLVNNKINKIENI |
| | (SEQ ID NO: 38) |
| Mut29A | IPSGTTILNCFHDVLSGKLSGGSPGVP |
| | (SEQ ID NO: 39) |
| Mut29B | SFACLRQPSQGPTVGVKGGAAGGGYAQ |
| | (SEQ ID NO: 40) |
| Mut30 | PSKPSFQEFVDWENVSPELNSTDQPFL |
| | (SEQ ID NO: 27) |
| Mut36 | CGTAFFINFIAIYHHASRAIPFGTMVA |
| | (SEQ ID NO: 41) |
| Mut44 | EFKHIKAFDRTFANNPGPMVVFATPGM |
| | (SEQ ID NO: 28) |
| Mut45 | ECRITSNFVIPSEYWVEEKEEKQKLIQ |
| | (SEQ ID NO: 42) |
| Mut48 | SHCHWNDLAVIPAGVVHNWDFEPRKVS |
| | (SEQ ID NO: 43) |
| Mut50 | GFSQPLRRLVLHVVSAAQAERLARAEE |
| | (SEQ ID NO: 44) |

Briefly, the above listed neoantigens were weighed one after another in a single sterile container. T-helper peptide A16L (AQYIKANSKFIGITEL; SEQ ID NO: 1) was also added to the weighed powder mixture at half the weight of the neoantigen. The entire neoantigen/T-helper powder mixture was then dissolved in sodium hydroxide, 100 mM solution at a concentration of 2 mg/gm per neoantigen and A16L at 1 mg/gm by vortexing for a minute and sonication for 2 minutes. To the prepared neoantigen stock solution, an equal portion (1:1) of sodium acetate, 100 mM, pH 6.0 solution was added quickly to attain a 1 mg/gm concentration per neoantigen and 0.5 mg/gm for A16L peptide antigen. Separately, a nucleotide adjuvant stock was prepared at a concentration of 10 mg/mL by mixing a DNA based polyI:C polynucleotide adjuvant (dIdC) in sterile water and used in the formulation at 0.2 mg/mL concentration.

To the diluted neoantigen stock solution, a previously prepared stock of DOPC:cholesterol (10:1 w/w) sized lipid vesicle particles in sodium acetate, 50 mM, pH 6.0 (size <100 nm with a pdi <0.1) was added and mixed well using a magnetic stir bar/shaker at 300 RPM for 3 minutes. Next, the nucleotide adjuvant stock was added to the neoantigen sized lipid vesicle particles and mixed well by shaking/stirring at 300 RPM for 2 minutes. The pH was adjusted to 10±1 with glacial acetic acid. The size and pH of the sized lipid vesicle particle/neoantigen mixture was confirmed using a DLS ZETASIZER NANO-S particle size analyzer before proceeding to the next step.

Sterile filtration was performed on the sized lipid vesicle particle/neoantigen mixture by passing the mixture through a Millipak-20 sterilizing grade Durapore membrane. The filtration was done using medical grade nitrogen gas pressure <55 psi. In brief, the sized lipid vesicle particle/neoantigen mixture was loaded into a pressure vessel fitted with a Millipak-20 PVDF 0.22 μm membrane and passed using nitrogen gas at 40 psi to complete the sterile filtration.

After sterile filtration, an appropriate volume (e.g. 1.0 ml/vial) of the sterile sized lipid vesicle particle/neoantigen mixture was aseptically filled into sterile vials to provide the concentration tabulated below when the dried preparation is subsequently solubilized in Montanide® ISA 51.

TABLE 6
Formulation components and concentration

| | Standard formulation, mg/mL |
|---|---|
| Components | (final oil solubilized vaccine product) |
| Lipid mixture | 132.0 |
| Neoantigen peptide(s) | 0.5 (each peptide) |
| T-helper peptide epitope | 0.25 |
| Oligo nucleotide Adjuvant | 0.4 |
| Sodium acetate | 100 mM, pH 10 ± 1 |

The vials and other product specifications used for the lyophilization are as detailed in the table below.

TABLE 7
Primary packaging specifications (Product Containers)

| Specifications for | Description |
|---|---|
| Vial | Vial 2 mL or 3 mL 13 MM FTN BB LYO PF |
| Stopper | Fluorotec Lyophilization Closure, 13 MM, |
| | single vented stoppers |
| Seal | West-Spectra Flip-Off 13 mm Seal |
| Autoclave bags | Fisher brand, Instant sealing sterilization |
| | pouch (19 × 33 cm), Cat#01-812-55 |
| | Fisher brand, Instant sealing sterilization |
| | pouch (30 × 46 cm), Cat# 01-812-58 |

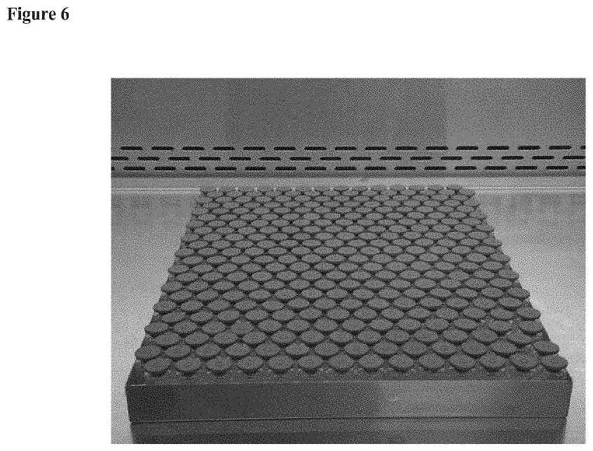

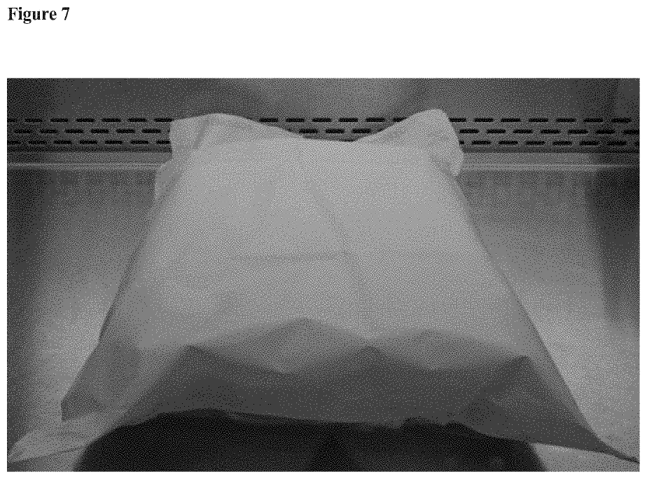

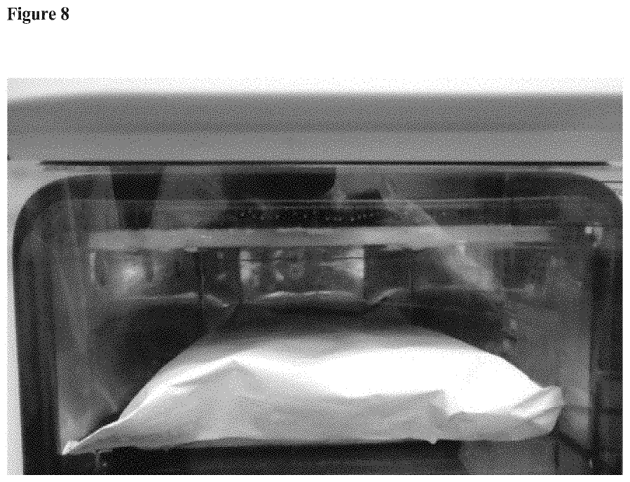

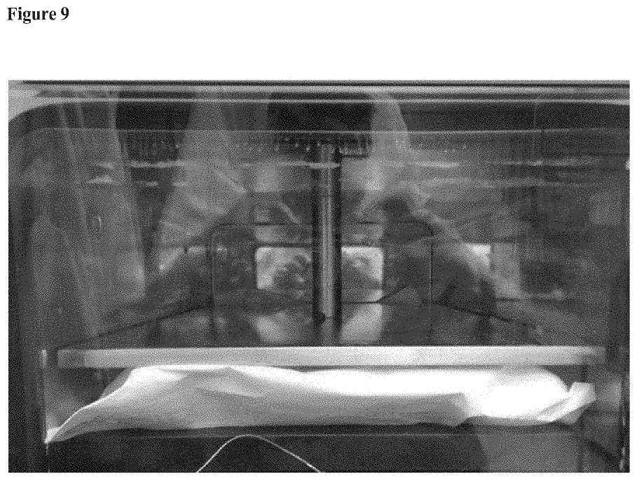

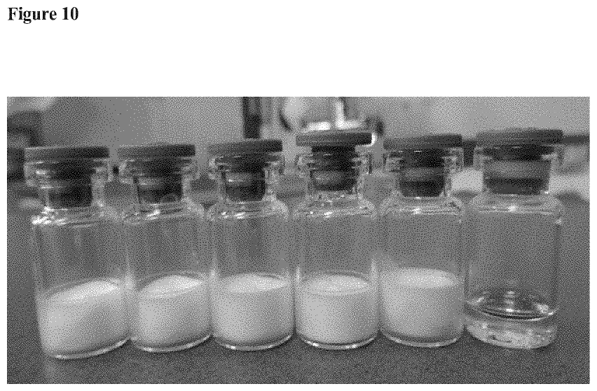

For lyophilization, the aseptically filled vials were loaded and sealed into sterile autoclave bags under sterile conditions, and then lyophilized (FIGS. 6-10). The lyophilization was performed over a period of approximately 75 hours, with a primary drying at −30° C. for 50 hours and a secondary drying at +25° C. for 16 hours. More specifically, the lyophilization was performed according to the following protocol:

TABLE 8
Exemplary lyophilization (freeze-dry) cycle

| | | Shelf Temp. | | Step | Cond. Temp |
|---|---|---|---|---|---|
| | | Set Point | | Time | Set Point |
| Step | Step Type | (° C.) | Vacuum | hh:mm | (° C.) |
| 1 | Loading | 20 | | | |
| 2 | Freezing | 5 | | 00:20 | |
| 3 | Freezing | 5 | | 02:00 | |
| 4 | Freezing | −40 | | 01:00 | |
| 5 | Freezing | −40 | | 02:00 | |
| 6 | Evacuation | −40 | 100 mT | — | −75 |
| 7 | Drying | −40 | 100 mT | 02:30 | −75 |
| 8 | Primary Drying | −30 | 100 mT | 08:20 | −75 |
| 9 | Primary Drying | −30 | 100 mT | 50:00 | −75 |
| 10 | Sec. Drying | 25 | 100 mT | 16:40 | −75 |
| 11 | Sec. Drying | 25 | 100 mT | 5:00 | −75 |

The dried lipid/therapeutic agent preparation was then solubilized in Montanide® ISA 51 to formulate the components as a water-free composition. The table below shows the molar ratios of the neoantigens, T-helper peptide and oligonucleotide adjuvant compared to the lipid component.

TABLE 9
Molar concentration of 14 neoantigens, A16L and oligonucleotide
adjuvant compared to lipid concentration

| | | Molar | Molar |
|---|---|---|---|
| | | concentration | concentration |
| | | of components | of lipid |
| | | in final oil | in final oil |
| | | solubilized | solubilized |
| | Component | vaccine | vaccine |
| Component | Details | product | product |
| 14 Neoantigens | Mut-17, 20, 22, | 2.32 mM | 183 mM |
| | 24, 25, 28, 29A, | | |
| | 29B, 30, 36, 44, | | |
| | 45, 48, 50 | | |
| T-Helper Peptide | A16L | 0.139 mM | |
| Adjuvant | Oligonucleotide | 0.051 mM | |

| | |
|---|---|
| Antigen(s)/Adjuvant versus | 2.51 mM:183 mM (1:73 ratio) |
| Lipid molar ratio | |

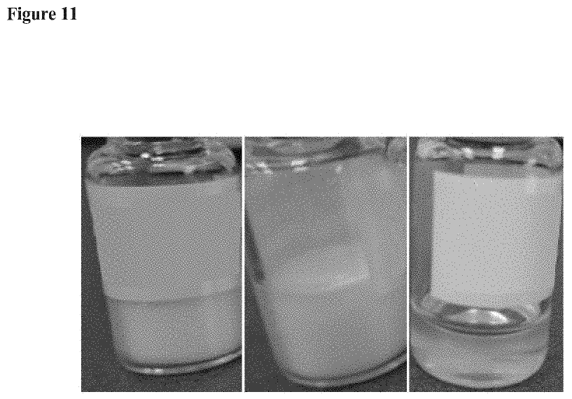

The freeze-dried product resulted in a homogenously dried cake and upon solubilization in Montanide® ISA 51 formed a clear to slightly hazy solution (see FIG. 11)

### Example 5

The composition prepared as described in Example 4, containing 14 neoantigens and the A16L T-helper peptide, was subjected to HPLC analysis to evaluate the peptide stability before and after freeze-drying. The HPLC instrument conditions and the gradient profile tested are shown the tables below.

TABLE 10
HPLC Instrument Conditions

| HPLC Model | Agilent 1100 Series (G1322A Vacuum |
|---|---|
| | Degasser, G1311A Quaternary Pump, |
| | G1329A Autosampler, G1316A |
| | Column Compartment, G1315B DAD, |
| | G1330A Autosampler Thermostat) |
| HPLC Method | NEO.M (or clone thereof) |
| HPLC Column | PhenomenexAeris PEPTIDEXB-C18, |
| | 3.6 μm, 250 × 4.6 mm, 100 Å with |
| | guard cartridge |
| Mobile Phase A | 0.1% TFA in water |
| Mobile Phase B | 0.1% TFA in acetonitrile |
| Mobile Phase C | Methanol |
| Flow Rate | 1.0 mL/min |
| Injection Volume | 50 μL |
| Column Temperature | 45° C. |
| Autosampler Temperature | 5° C. |
| Detector | UV |
| Detection (nm) | 215 |
| Run Time (minutes) | 90 |
| Post Time (minutes) | 10 |

TABLE 11
Gradient Profile

| Step | | | | |
|---|---|---|---|---|
| Number | Time (minutes) | % MPA | % MPB | % MPC |
| 1 | 0.0 | 83.0 | 17.0 | 0.0 |
| 2 | 12.0 | 75.0 | 25.0 | 0.0 |
| 3 | 25.0 | 74.0 | 26.0 | 0.0 |
| 4 | 40.0 | 70.0 | 30.0 | 0.0 |
| 5 | 75.0 | 55.0 | 45.0 | 0.0 |
| 6 | 75.01 | 0.0 | 0.0 | 100.0 |
| 7 | 90 | 0.0 | 0.0 | 100.0 |

Experimental Conditions:
IPQC Solutions: Dilute the IPQC solutions with Methanol and then with Mobile Phase A, e.g. as follows:
   100 mg In-Process Testing Solution
   +450 μL Methanol, mix
   +450 μL Mobile Phase A, mix
   Mix by vortexing. Samples containing lipids will be slightly hazy. Dilution factor=10×.
Lyophilisates (for Release and Stability Testing): Solubilize the lyophilisate with Ultrapure lab water. Add 3-4 glass beads and vortex vigorously for 1 minute to assure complete homogenization. Dilute the solution with Methanol and subsequently with Mobile Phase A, e.g. as follows:
   100 mg In-Process Testing Solution
   +450 μL Methanol, mix
   +450 μL Mobile Phase A, mix
   Mix by vortexing. The sample preparations will be slightly hazy. Dilution factor=20×.

An HPLC chromatogram of a standard containing the 14 neoantigens and the A16L T-helper peptide is shown in FIG. 12.

The neoantigen and A16L materials prepared according to Example 4 were characterized quantitatively using the HPLC method both before and after freeze-drying. The HPLC chromatogram showing the 14 neoantigens and A16L T-helper peptide before freeze-drying is shown in FIG. 13.

The HPLC chromatograms obtained after freeze-drying for T=0, T=1M and T=3M are shown in FIGS. 14-16, respectively. The dried preparations were solubilized in water for HPLC analysis. T=0 is the day in which the samples were removed from the freeze-dryer. T=1M and T=3M are 1 month and 3 months after T=0, respectively, with the dried preparation having been stored at −20° C. The calculated peptide recovery is depicted in the table below.

TABLE 12
Neoantigen and A16L recovery before and after freeze-drying

| | Amount from non-validated HPLC assay (mg/mL) | | | | | | | | | | | | | | |
|---|---|---|---|---|---|---|---|---|---|---|---|---|---|---|---|
| Sample | Mut | Mut | | Mut | Mut | Mut | Mut | Mut | Mut | Mut | Mut | Mut | Mut | Mut | Mut |
| Time | 24 | 29b | A16L | 28 | 45 | 22 | 20 | 44 | 48 | 25 | 30 | 50 | 29a | 17 | 36 |
| Before | 0.56 | 0.56 | 0.25 | 0.53 | 0.47 | 0.54 | 0.42 | 0.51 | 0.47 | 0.56 | 0.53 | 0.42 | 0.54 | 0.51 | 0.47 |
| freeze | | | | | | | | | | | | | | | |
| dry | | | | | | | | | | | | | | | |
| After | 0.44 | 0.48 | 0.25 | 0.50 | 0.44 | 0.51 | 0.41 | 0.47 | 0.38 | 0.54 | 0.51 | 0.42 | 0.38 | 0.27 | 0.19 |
| freeze | | | | | | | | | | | | | | | |
| dry | | | | | | | | | | | | | | | |
| T = 0 | | | | | | | | | | | | | | | |
| After | 0.38 | 0.44 | 0.23 | 0.50 | 0.42 | 0.47 | 0.47 | 0.44 | 0.37 | 0.50 | 0.51 | 0.44 | 0.34 | 0.23 | 0.25 |
| freeze | | | | | | | | | | | | | | | |
| dry | | | | | | | | | | | | | | | |
| T = 1M | | | | | | | | | | | | | | | |
| After | 0.37 | 0.48 | 0.27 | 0.51 | 0.46 | 0.54 | 0.54 | 0.50 | 0.41 | 0.56 | 0.56 | 0.46 | 0.35 | 0.26 | 0.31 |
| freeze | | | | | | | | | | | | | | | |
| dry | | | | | | | | | | | | | | | |
| T = 3M | | | | | | | | | | | | | | | |

The results shown in FIGS. 13-16, and Table 12 above, indicate that the peptides were stable in the dried lipid/therapeutic agent preparation when prepared by the described method, and peptide concentration was within the set specifications. (Set specifications: 0.40-0.60 mg/mL for the neoantigens and 0.2-0.3 mg/mL for A16L.)

### Example 6

Pathogen free, C57BL/6NCrl mice, 6-8 weeks of age, were purchased from Charles River Laboratories (St. Constant, PQ) and housed according to institutional guidelines with water and food ad libitum under filter controlled air circulation. The neoantigens were synthesized by Genscript and are listed above in Table 5.

Mice in group 1 (n=5) were vaccinated with 100 microliters of an aqueous, non-depot forming vaccine containing the 14 neoantigens, each at 50 microgram dose, A16L T-helper peptide at 50 microgram dose, DNA based poly I:C at 40 microgram dose in sodium acetate buffer, pH 10.5.

Mice in group 2 (n=5) were vaccinated with 100 microliters of an oil based, depot forming vaccine containing the 14 neoantigens, each at 50 microgram dose, A16L T-helper peptide at 50 microgram dose, DNA based poly I:C at 40 microgram dose, 12 milligrams of DOPC, 1.2 milligrams of cholesterol in ISA51 VG oil.

Eight days after vaccination, mice were terminated and spleens collected. Splenocytes were stimulated in an IFN-gamma ELISPOT plate (BD Biosciences) with syngeneic dendritic cells unloaded (background; BG) or loaded with an irrelevant peptide (RAHYNIVTF; SEQ ID NO: 9) or each individual neoantigen (17, 20, 22, 24, 25, 28, 29A, 29B, 30, 36, 44, 45, 48, 50). After 18 hours of culture, the plates were developed and the number of spot forming units (SFU) counted using Immunospot Reader (C.T.L.). The results are shown in FIG. 17 as average response±SEM. Statistical analysis was performed by 2-way ANOVA with Bonferroni post test comparing group responses to each peptide.

The results demonstrate that the oil based formulation enhances immune responses after a single immunization better than the aqueous formulation.

### Example 7

The Batch #4c (survivin) and Batch #4a (rPA) compositions from Example 1 were analyzed by small angle x-ray scattering technique (SAXS) to determine the size and shape of the lipid-based structures present in the hydrophobic carrier when the compositions were prepared with sized lipid vesicle particles.

The SAXS patterns were collected at University of Sherbrooke, QC, Canada, with a Bruker AXS Nanostar system, equipped with a Microfocus Copper Anode at 45 kV/0.65 mA, MONTAL OPTICS and a VANTEC 2000 2D detector at 27.3 cm distances from the samples. The distances were calibrated with a Silver Behenate standard prior to the measurements. The samples were injected into 0.6 mm diameter special glass capillaries, sealed, and placed at predetermined positions. The positioning fine tuning was done by nanography; a 2 second per step scan sweep on X and Y to find the exact position of the samples. The scattering intensities were treated with Primus GNOM 3.0 programs from ATSAS 2.3 software.

Scans were measured for (1) Montanide ISA 51 VG (blank control), (2) Batch #4c composition and (3) Batch #4a composition. Scans were performed with 800 sec. exposure for the Montanide ISA 51 VG sample, Batch #4c sample and Batch #4a sample. The Montanide ISA 51 VG was mathematically subtracted from the Batch #4c and Batch #4a samples to determine the particle size and shape by a pair-distance distribution function. The gaussian curve shape is typical for a spherical particle.

FIG. 18 shows the results for Montanide ISA 51 VG (blank control). No particle structures were observed. As such, no evaluation of particle size was performed.

FIG. 19 shows the results for the Batch #4c composition (survivin). The image indicates that the lipids form a single layer assembly. As shown by the particle size evaluation, the Dmax particle size is about 5.8 nm and the shape estimated by SAXS is spherical. This corresponds to the size of reverse micelles.

FIG. 20 shows the results for the Batch #4a composition (rPA). The image indicates that the lipids form a single layer assembly. As shown by the particle size evaluation, the Dmax particle size is about 4.0 nm and the shape estimated by SAXS is spherical. This corresponds to the size of reverse micelles.

### Example 8

The effect of preparing sized lipid vesicle particles with DOPC/Cholesterol or S100 Lecithin in MS80 oil carrier was studied. MS80 oil is a mixture of mineral oil (Sigma Aldrich) and Span80 (Fluka), purchased separately and mixed.

Sized DOPC/Cholesterol Formulations in MS80

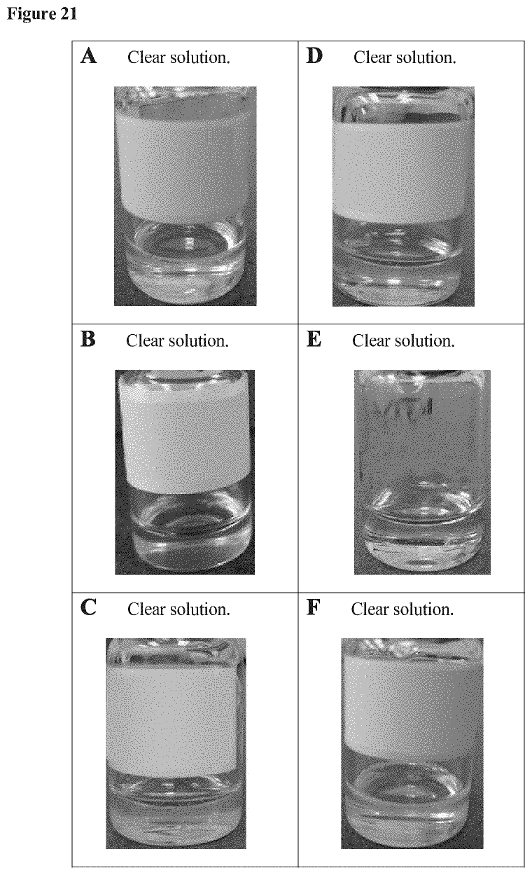

Batch #10: To prepare a dried preparation comprising recombinant Protective Antigen (rPA; same antigen as in Batch #4a), a 10:1 (w:w) homogenous mixture of DOPC/cholesterol (Lipoid GmbH, Germany) was added to sodium phosphate, 100 mM, pH 7.0 with shaking at 300 RPM for about 1 hour. The mixture was then sized by passing the material 25 times through a 0.2 μm polycarbonate membrane and then 10 times through a 0.1 μm polycarbonate membrane to attain a particle size of ≤120 nm with a pdi of ≤0.1. The rPA antigen (0.25 mg/mL) and a lipopeptide adjuvant (R-Pam3Cys; at 0.02 mg/ml) were added with shaking at 300 RPM for about 15 minutes. The mixture was then subjected to serial sterile filtration by passing the mixture through Millipak-20 PVDF 0.22 μm membranes. The sterile solution was aliquoted into vials and freeze-dried to prepare the dried lipid/therapeutic agent preparation. To the dried preparation, 700 μl of MS80 oil was added with soaking for 5 minutes followed by shaking for 2 minutes. As shown in FIG. 21A, a clear solution was obtained.

Batch #11: To prepare a dried preparation comprising RSV peptide antigen (SHe A peptide; NKLCEYNVFHNKTFELPRARVNT; SEQ ID NO: 2) in dimer form, a 10:1 (w:w) homogenous mixture of DOPC/cholesterol (Lipoid GmbH, Germany) was added to sodium phosphate, 100 mM, pH 6.0 with shaking at 300 RPM for about 1 hour. The mixture was then sized by passing the material 25 times through a 0.2 μm polycarbonate membrane and then 10 times through a 0.1 μm polycarbonate membrane to attain a particle size of ≤120 nm with a pdi of ≤0.1. The RSV peptide antigen dimer (0.25 mg/mL) and a lipopeptide adjuvant (R-Pam3Cys; at 0.02 mg/ml) were added with shaking at 300 RPM for about 15 minutes. The mixture was then subjected to sterile filtration by passing the mixture through a single Millipak-20 PVDF 0.22 μm membrane. The sterile solution was aliquoted into vials and freeze-dried to prepare the dried lipid/therapeutic agent preparation. To the dried preparation, 450 μl of MS80 oil was added with soaking for 5 minutes followed by shaking for 2 minutes. As shown in FIG. 21B, a clear solution was obtained.

Sized S100 Lecithin Formulations in MS80

Batch #12: To prepare a dried preparation comprising RSV peptide antigen (SHe A peptide; NKLCEYNVFHNKTFELPRARVNT; SEQ ID NO: 2) in dimer form, Lipoid S100 lecithin (Lipoid GmbH, Germany) was added to sodium acetate, 100 mM, pH 6.0 with shaking at 300 RPM for about 1 hour. The mixture was then sized by passing the material 25 times through a 0.2 μm polycarbonate membrane and then 10 times through a 0.1 μm polycarbonate membrane to attain a particle size of ≤120 nm with a pdi of ≤0.1. The RSV peptide antigen dimer (0.25 mg/mL) and a lipopeptide adjuvant (R-Pam3Cys; at 0.02 mg/ml) were added with shaking at 300 RPM for about 15 minutes. The mixture was then subjected to sterile filtration by passing the mixture through a single Millipak-20 PVDF 0.22 μm membrane. The sterile solution was aliquoted into vials and freeze-dried to prepare the dried lipid/therapeutic agent preparation. To the dried preparation, 450 μl of MS80 oil was added with soaking for 5 minutes followed by shaking for 2 minutes. As shown in FIG. 21C, a clear yellow solution was obtained. The solution is yellow because the 5100 (soy lecithin) material is yellow in colour and hence the solubilized composition is also yellow.

Batch #13: To prepare a dried survivin antigen preparation with S100 lecithin vesicle particles, Lipoid S100 lecithin (Lipoid GmbH, Germany) was added to sodium acetate, 100 mM, pH 9.5 with shaking at 300 RPM for about 1 hour. The mixture was then sized by passing the material 25 times through a 0.2 μm polycarbonate membrane and then 10 times through a 0.1 μm polycarbonate membrane to attain a particle size of ≤120 nm with a PDI of ≤0.1. Five synthetic survivin peptide antigens (FTELTLGEF (SEQ ID NO: 4), LMLGEFLKL (SEQ ID NO: 5), RISTFKNWPK (SEQ ID NO: 6), STFKNWPFL (SEQ ID NO: 7) and LPPAWQPFL (SEQ ID NO: 8); each at 0.5 mg/mL) and a dIdC oligonucleotide adjuvant (0.2 mg/mL) were added with shaking at 300 RPM for about 15 minutes. A T-helper peptide (A16L peptide; AQYIKANSKFIGITEL (SEQ ID NO: 1); at 0.25 mg/mL) was then added to the sized lipid vesicle particle/survivin antigen-adjuvant mixture with shaking at 300 RPM for about 5 minutes. The mixture was then subjected to serial sterile filtration by passing the mixture through Millipak-20 PVDF 0.22 μm membranes. The sterile solution was aliquoted into vials and freeze dried to prepare the dried lipid/therapeutic agent preparation. To the dried preparation, 700 μl of MS80 oil was added with soaking for 5 minutes followed by shaking for 2 minutes. As shown in FIG. 21D, a clear yellow solution was obtained. Again, the solution is yellow because the S100 (soy lecithin) material is yellow in colour and hence the solubilized composition is also yellow.

Batch #14: To prepare a dried preparation comprising a malaria antigen, the procedure above for Batch #13 was repeated with the exception that the survivin peptide antigens and T-helper epitope were replaced with malaria VLP antigen (circumsporozoite (CS) T and B cell epitopes displayed on the woodchuck hepatitis virus core antigen (WHcAg); at 0.2 mg/mL). To the dried preparation, 545 μl of MS80 oil was added with soaking for 5 minutes followed by shaking for 2 minutes. As shown in FIG. 21E, a clear yellow solution was obtained. Again, the solution is yellow because the S100 (soy lecithin) material is yellow in colour and hence the solubilized composition is also yellow.

Sized S100 Lecithin/Cholesterol Formulations in MS80

Batch #15: To prepare a dried preparation comprising HIV peptide antigens AMQ9 (AMQMLKETI; SEQ ID NO: 12) and RGP10 (RGPGRAFVTI; SEQ ID NO: 11) purchased from Genscript (Piscataway, N.J.), a 10:1 (w:w) homogenous mixture of Lipoid S100 lecithin/cholesterol (Lipoid GmbH, Germany) was added to sodium acetate, 100 mM, pH 9.5 with shaking at 300 RPM for about 1 hour. The mixture was then sized by passing the material 25 times through a 0.2 μm polycarbonate membrane and then 10 times through a 0.1 μm polycarbonate membrane to attain a particle size of ≤120 nm with a pdi of ≤0.1. The HIV peptide antigens (0.5 mg/mL each) and a dIdC oligonucleotide adjuvant (0.2 mg/mL) were added with shaking at 300 RPM for about 15 minutes. A T-helper peptide (A16L peptide; AQYIKANSKFIGITEL (SEQ ID NO: 1); at 0.25 mg/mL) was then added to the sized lipid vesicle particle/HIV antigen-adjuvant mixture with shaking at 300 RPM for about 5 minutes. The mixture was then subjected to serial sterile filtration by passing the mixture through Millipak-20 PVDF 0.22 μm membranes. The sterile solution was aliquoted into vials and freeze-dried to prepare the dried lipid/therapeutic agent preparation. To the dried preparation, 700 μl of MS80 oil was added with soaking for 5 minutes followed by shaking for 4 minutes. As shown in FIG. 21E, a clear yellow solution was obtained. Again, the solution is yellow because the S100 (soy lecithin) material is yellow in colour and hence the solubilized composition is also yellow.

The characteristics of the resultant compositions after solubilization are described in the table below and in FIG. 21.

TABLE 13
Product Characteristics—DOPC/cholesterol
or S100 Lecithin in MS80

| | | | Sized Lipid Vesicle |
|---|---|---|---|
| Batch | Antigen | Lipids | Particle Composition |
| 10 | rPA | DOPC/Chol | Figure 21A— |
| | | | Clear solution. |
| 11 | RSV | DOPC/Chol | Figure 21B— |
| | | | Clear solution. |
| 12 | RSV | S100 Lecithin | Figure 21C— |
| | | | Clear yellow solution. |
| 13 | Survivin | S100 Lecithin | Figure 21D— |
| | | | Clear yellow solution. |
| 14 | Malaria | S100 Lecithin | Figure 21E— |
| | | | Clear yellow solution. |
| 15 | HIV | S100/Chol | Figure 21F— |
| | | | Clear yellow solution. |

The results demonstrate that compositions prepared using sized lipid vesicle particles prepared with DOPC/Cholesterol, S100 Lecithin and S100 Lecithin/Cholesterol all provide a clear solution upon solubilization in an MS80 oil carrier. This exemplifies that the surfactant in Montanide ISA 51 (i.e. mannide oleate) is not critical to obtaining a suitable pharmaceutical composition.

### Example 9

Dried Lipid/Therapeutic Agent Preparation

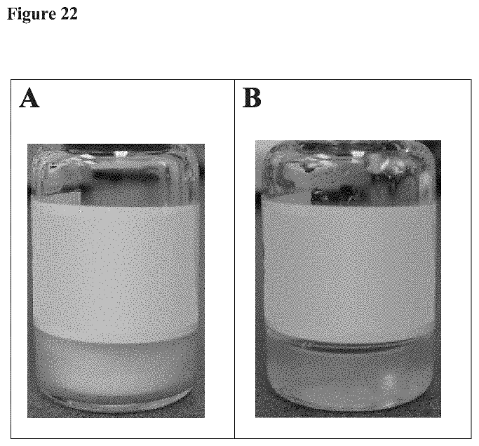

To prepare a dried lipid/therapeutic agent preparation, a 10:1 (w:w) homogenous mixture of DOPC and cholesterol (Lipoid GmbH, Germany) was added to sodium acetate, 100 mM, pH 9.5 at a concentration of 132 mg/ml, with shaking at 300 RPM for about 1 hour to form lipid vesicle particles. The mixture was then sized by passing the material 25 times through a 0.2 μm polycarbonate membrane and 10 times through a 0.1 μm polycarbonate membrane to attain a mean particle size of ≤120 nm with a pdi of ≤0.1. To the sized lipid vesicle particles, a small molecule drug (cyclophosphamide) was added at a concentration of 2.0 mg/mL, with shaking at 300 RPM for about 15 minutes. The mixture was then subjected to serial sterile filtration by passing the mixture through Millipak-20 PVDF 0.22 μm membranes. The sterile solution was aliquoted into vials and freeze-dried to prepare the dried lipid/therapeutic agent preparation. This dried lipid/therapeutic agent preparation is hereinafter referred to as Batch #16. As shown in FIG. 22, the appearance of the dried preparation looks good, i.e. it is dry, white and non-collapsed.

Solubilization in a Hydrophobic Carrier

The dried lipid/therapeutic agent preparations of Batch #16 was solubilized in an oil diluent (i.e. Montanide® ISA 51 VG) to provide a final composition with 4.0 mg/mL of cyclophosphamide and 132 mg/ml DOPC/cholesterol (10:1 w/w).

As shown in FIG. 22, by using the methods disclosed herein it was possible to solubilize the cyclophosphamide in a hydrophobic carrier to obtain a clear to slightly hazy solution that was free of particulate.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

As used herein, whether in the specification or the appended claims, the transitional terms “comprising”, “including”, “carrying”, “having”, “containing”, “involving”, and the like are to be understood as being inclusive or open-ended (i.e., to mean including but not limited to), and they do not exclude unrecited elements, materials or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials or steps and to those that do not materially affect the basic characteristic(s) of the invention disclosed and/or claimed herein.

### REFERENCES

1) Akbarzadeh, A.; Rezaei-Sadabady, R.; Davaran, S.; Joo, S. W.; Zarghami, N.; Hanifehpour, Y.; Samiei, M.; Kouhi, M.; and Nejati-Koshki, K. (2013) Liposome: classification, preparation, and applications, Nanoscale Res Lett. 8(1), 102.
2) Banga, A. K. Therapeutic Peptides and Proteins, Formulation, Processing and Delivery Systems (Lancaster, Pa.: Technomic Publishing Co., 1995).
3) Bobst, A. M.; Langemeier, P. W.; Torrence, P. F.; De Clercq, E. (1981) Interferon Induction by Poly(inosinic acid).Poly(cytidylic acid) Segmented by Spin-Labels, Biochemistry 20(16), 4798-4803.
4) Caruthers, M. H.; Beaucage, S. L.; Efcavitch, J. W.; Fisher, E. F.; Matteucci, M. D.; Stabinsky, Y. (1980) New chemical methods for synthesizing polynucleotides, Nucleic Acids Res. Symp. Ser. (7), 215-223.
5) Castle, J. C.: Kreiter, S.; Diekmann, J.; Lower, M.; van de Roemer, N.; de Graaf, J.; Selmi, A.; Diken, M.; Boegel, S.; Paret, C.; Koslowski, M.; Kuhn, A. N.; Britten, C. M.; Huber, C.; Turesi, O.; and Sahin, U. (2012) Exploiting the Mutanome for Tumor Vaccination. Cancer Res, 72(5): 1081-1091.
6) Celis, E.; Ou, D.; Otvos, L. Jr. (1988) Recognition of hepatitis B surface antigen by human T lymphocytes. Proliferative and cytotoxic responses to a major antigenic determinant defined by synthetic peptides, J. Immunol. 140, 1808-1815.
7) Chong, P.; Zobrist, G.; Sia, C.; Loosmore, S.; Klein, M. (1992) Identification of T- and B-cell epitopes of the S2 and S3 subunits of pertussis toxin by use of synthetic peptides, Infect Immun. 60, 4640-4647.
8) Coligan et al., ed. Current Protocols in Immunology, Wiley Interscience, 2007
9) Collins, P. L.; Olmsted, R. A.; Johnson, P. R. (1990) The small hydrophobic protein of human respiratory syncytial virus: comparison between antigenic subgroups A and B, J Gen Virol. 71(7), 1571-6.
10) Demotz, S.; Lanzavecchia, A.; Eisel, U.; Niemann, H.; Widmann, C.; Corradin, G. P. (1989) Delineation of several DR-restricted epitopes in tetanus toxin, J. Immunol. 142, 394-402.
11) Diethelm-Okita, B. M.; Okita, D. K.; Banaszak, L.; Conti-Fine, B. M. (2000) Universal epitopes for human CD4+ cells on tetanus and diphtheria toxins, J. Infect. Dis. 181, 1001-1009.
12) Flynn, J. L. (1994) Recombinant BCG as an antigen delivery system, Cell. Mol. Biol. 40 (suppl. 1), 31-36.
13) Frezard, F. (1999) Liposomes: from biophysics to the design of peptide vaccines, Braz. J. Med. Bio. Res. 32, 181-189.
14) Greene, J. J.; Alderfer, J. L.; Tazawa, I.; Tazawa, S.; Ts'o, P. O.; O'Malley, J. A.; Carter, W. A. (1978) Interferon induction and its dependence on the primary and secondary structure of poly(inosinic acid).poly(cytidylic acid), Biochemistry 17(20), 4214-4220.
15) Gregoriadis G. (1990) Immunological adjuvants: a role for liposomes, Immunol. Today 11, 89-97.
16) Hacohen, N.; Wu, C. J.; Fritsch, E. F.; Combination therapy with Neoantigen Vaccine, US20160339090A1.
17) Hagstrom, N. J.; Couto, L. B.; Scallan, C.; Burton, M.; McCleland, M. L.; Fields, P. A.; Arruda, V. R.; Herzog, R. W. and High, K. A. (2000) Blood, 95, 2536-2542.
18) Hendrix, C. W.; Margolick, J. B.; Petty, B. G.; Markham, R. B.; Nerhood, L.; Farzadegan, H.; Ts'o, P. O.; Lietman, P. S. (1993) Biologic effects after a single dose of poly(I):poly(C12U) in healthy volunteers, Antimicrob. Agents Chemother. 37(3), 429-435.
19) High, N.; Mounier, J.; Prevost, M. C.; Sansonetti, P. J. (1992) IpaB of Shigella flexneri causes entry into epithelial cells and escape from the phagocytic vacuole, EMBO J 11, 1991-1999.
20) Horn, T.; Vasser, M. P.; Struble, M. E.; Crea, R. (1980) Synthesis of oligonucleotides on cellulose. Part II: Design and synthetic strategy to the synthesis of 22 oligodeoxynucleotides coding for gastric inhibitory polypeptide (GIP), Nucleic Acids Symp. Ser. (7), 225-232.
21) Kawaoka, Y.; Yamnikova, S.; Chambers, T. M.; Lvov, D. K.; Webster, R. G. (1990) Molecular characterization of a new hemagglutinin, subtype H14, of influenza A virus, Virology 179(2), 759-767.
22) Kieny, M. P.; Lathe, R.; Drillien, R.; Spehner, D.; Skory, S.; Schmitt, D.; Wiktor, T.; Koprowski, H. and Lecocq, J. P. (1984) Expression of rabies virus glycoprotein from a recombinant vaccinia virus, Nature 312, 163-166.
23) Levey, D. L.; Castle, J. C.; Stein, R. B. and Buell, J. S. Vaccines for treatment and prevention of cancer, US20160331821A1.
24) Li, X.; Eastman, E. M.; Schwartz, R. J.; and Draghia-Akli, R. (1999) Synthetic muscle promoters: activities exceeding naturally occurring regulatory sequences, Nat. Biotechnol 17, 241-245.
25) Li, Z. L. and Paulin, D. (1991) High level desmin expression depends on a muscle-specific enhancer, J. Biol. Chem. 266, 6562-6570.
26) Li, Z. L. and Paulin, D. (1993) Different factors interact with myoblast-specific and myotube-specific enhancer regions of the human desmin gene, J. Biol. Chem. 268, 10403-10415.
27) Medaglini, D.; Pozzi, G.; King, T. P. and Fischetti, V. A. (1995) Mucosal and systemic immune responses to a recombinant protein expressed on the surface of the oral commensal bacterium Streptococcus gordonii after oral colonization, Proc. Natl. Acad. Sci. USA 92, 6868-6872.
28) Mekalanos, J. J.; Swartz, D. J.; Pearson, G. D. N.; Harford, N.; Groyne, F. and de wilde, M. (1983) Cholera toxin genes: nucleotide sequence, deletion analysis and vaccine development, Nature 306, 551-557.
29) Merrifield, B. (1997) Concept and early development of solid-phase peptide synthesis, Methods Enzymol. 289, 3-13.
30) Moyle, P. M.; Toth. I. (2008) Self-adjuvanting lipopeptide vaccines, Curr. Med. Chem. 15(5), 506-516.
31) Mullard A. (2016) The cancer vaccine resurgence, Nat Rev Drug Disc 15, 663-665.
32) Nakayama, K.; Kelly, S. M. and Curtis, R. III (1988) Construction of an Asd+ expression cloning vector: stable maintenance and high level expression of cloned genes in a Salmonella strain, Bio/Technology 6, 693-697.
33) Roberge, J. Y.; Beebe, X.; Danishefsky, S. J. (1995) A strategy for a convergent synthesis of N-linked glycopeptides on a solid support, Science 269(5221), 202-204.
34) Remington's Pharmaceutical Sciences, Mack Publishing Company, Easton, Pa., USA 1985.
35) Sizemore, D. R.; Branstrom, A. A.; and Sadoff, J. C. (1995) Attenuated Shigella as a DNA Delivery Vehicle for DNA-Mediated Immunimization, Science 270, 299-303.
36) Slingluff, C. L. Jr.; Yamshchikov, G.; Neese, P.; Galavotti, H.; Eastham, S.; Engelhard, V. H.; Kittlesen, D.; Deacon, D.; Hibbitts, S.; Grosh, W. W.; Petroni, G.; Cohen, R.; Wiernasz, C.; Patterson, J. W.; Conway, B. P.; Ross, W. G. (2001) Phase I trial of a melanoma vaccine with gp100(280-288) peptide and tetanus helper peptide in adjuvant: Immunologic and clinical outcomes, Clin Cancer Res. 7, 3012-3024.
37) Skwarczynski, M. and Toth, I. (2016) Peptide-based synthetic vaccines, Chem. Sci. 7, 842-854.
38) So, N. S. Y.; Ostrowski, M. A.; Gray-Owen, S. D. (2012) Vigorous Response of Human Innate Functioning IgM Memory B Cells upon Infection by Neisseria gonorrhoeae, J. Immunol. 188(8), 4008-4022.
39) Srivastava, P. (2015) Neoepitopes of Cancers: Looking Back, Looking Ahead, Cancer Immunol. Res. 3, 969-977.
40) Tartaglia, J.; Perkus, M. E.; Taylor, J.; Norton, E. K.; Audonnet, J. C.; Cox, W. I.; Davis, S. W.; van der Hoeven, J.; Meignier, B.; Riviere, M.; et al. (1992) NYVAC: a highly attenuated strain of vaccinia virus, Virology 188, 217-232.
41) Taylor, J.; Meignier, B.; Tartaglia, J.; Languet, B.; VanderHoeven, J.; Franchini, G.; Trimarchi, C.; and Paoletti E. (1995) Biological and immunogenic properties of a canarypox-rabies recombinant, ALVAC-RG (vCP65) in non-avian species, Vaccine 13, 539-549.
42) The United States Pharmacopoeia: The National Formulary (USP 24 NF19) published in 1999.
43) Webster, R. G.; Laver, W. G.; Air, G. M. Antigenic variation among type A influenza viruses, p. 127-168. In: Palese, P. & Kingsbury, D. W., eds. Genetics of influenza viruses. (New York: Springer-Verlag, 1983).
44) Zhenlin, L.; Lilienbaum, A.; Butler-Browne, G.; Paulin, D. (1989) Human desmin-coding gene: complete nucleotide sequence, characterization and regulation of expression during myogenesis and development, Gene 78, 243-254.

1. A method for preparing a dried preparation comprising lipids and a therapeutic agent, said method comprising the steps of:
(a) providing lipid vesicle particles having a mean particle size of ≤120 nm and a polydispersity index (PDI) of ≤0.1;
(b) mixing the lipid vesicle particles with at least one solubilized therapeutic agent to form a mixture; and
(c) drying the mixture formed in step (b) to form a dried preparation comprising lipids and a therapeutic agent.

2. The method of claim 1, wherein step (a) comprises sizing lipid vesicle particles to provide the lipid vesicle particles having a mean particle size of ≤120 nm and a PDI of ≤0.1.

3. The method of claim 1 or 2, wherein the mean particle size of the lipid vesicle particles is between about 80 nm and about 120 nm.

4. The method of any one of claims 1 to 3, wherein the mean particle size of the lipid vesicle particles is about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm or about 115 nm.

5. The method of any one of claims 1 to 4, wherein the mean particle size of the lipid vesicle particles is ≤100 nm.

6. The method of any one of claims 1 to 5, wherein the lipid vesicle particles comprise a synthetic lipid.

7. The method of claim 6, wherein the lipid vesicle particles comprise synthetic dioleoyl phosphatidylcholine (DOPC) or synthetic DOPC and cholesterol.

8. The method of claim 7, wherein the lipid vesicle particles comprise synthetic DOPC and cholesterol at a DOPC:cholesterol ratio of 10:1 (w/w).

9. The method of any one of claims 1 to 8, wherein the lipid vesicle particles of step (a) are prepared from a liposome precursor.

10. The method of claim 9, wherein the liposome precursor is Presome®.

11. The method of any one of claims 1 to 10, wherein the lipid vesicle particles are liposomes.

12. The method of claim 11, wherein the liposomes are unilamellar, multilamellar, multivesicular, or a mixture thereof.

13. The method of any one of claims 1 to 12, wherein the at least one therapeutic agent is solubilized in one or more of sodium acetate, sodium phosphate or sodium hydroxide.

14. The method of any one of claims 1 to 12, wherein the at least one therapeutic agent is solubilized in one or more of 0.1 M sodium hydroxide, 100 mM sodium acetate having a pH of 6.0±1.0, 100 mM sodium acetate having a pH of 9.5±1.0, 50 mM sodium phosphate having a pH of 7.0±1.0 or 100 mM sodium phosphate having a pH of 6.0±1.0.

15. The method of any one of claims 1 to 14, wherein the mixing of step (b) is performed in a sodium acetate or sodium phosphate solution.

16. The method of claim 15, wherein the mixing of step (b) is performed in 25-250 mM sodium acetate having a pH in the range of 6.0-10.5 or 25-250 mM sodium phosphate having a pH in the range of 6.0-8.0.

17. The method of claim 15, wherein the mixing of step (b) is performed in 50 mM sodium acetate having a pH of 6.0±1.0, 100 mM sodium acetate having a pH of 9.5±1.0, 50 mM sodium phosphate having a pH of 7.0±1.0 or 100 mM sodium phosphate having a pH of 6.0±1.0.

18. The method of claim 15, wherein the mixing of step (b) is performed in 50 mM sodium phosphate having a pH of 7.0, 100 mM sodium phosphate having a pH of 6.0, 50 mM sodium acetate having a pH of 6.0, or 100 mM sodium acetate having a pH of 9.5.

19. The method of any one of claims 1 to 18, wherein the therapeutic agent is a peptide antigen, a DNA or RNA polynucleotide that encodes a polypeptide, a hormone, a cytokine, an allergen, a catalytic DNA (deoxyribozyme), a catalytic RNA (ribozyme), an antisense RNA, an interfering RNA, an antagomir, a small molecule drug, a biologic drug, an antibody, or a fragment or derivative of any one thereof; or a mixture thereof.

20. The method of any one of claims 1 to 18, wherein the therapeutic agent is one or more peptide antigens.

21. The method of claim 20, wherein the one or more peptide antigens are 20-30 amino acids in length.

22. The method of claim 20 or 21, wherein the one or more peptide antigens are neoantigens.

23. The method of claim 20, wherein the one or more peptide antigens are derived from human papillomavirus (HPV), human immunodeficiency virus (HIV), respiratory syncytial virus (RSV), Bacillus anthracis, Plasmodium, or a survivin polypeptide.

24. The method of claim 23, wherein the one or more peptide antigens are FTELTLGEF (SEQ ID NO: 4), LMLGEFLKL (SEQ ID NO: 5), RISTFKNWPK (SEQ ID NO: 6), STFKNWPFL (SEQ ID NO: 7) or LPPAWQPFL (SEQ ID NO: 8); or any combination thereof.

25. The method of claim 23, wherein the one or more peptide antigens are NKLCEYNVFHNKTFELPRARVNT (SEQ ID NO: 2) and/or NKLSEHKTFCNKTLEQGQMYQINT (SEQ ID NO: 3).

26. The method of any one of claims 20 to 23, wherein step (b) comprises mixing five or more different solubilized peptide antigens with the lipid vesicle particles.

27. The method of claim 26, wherein step (b) comprises mixing up to 30 different solubilized peptide antigens with the lipid vesicle particles.

28. The method of claim 26, wherein step (b) comprises mixing 10, 11, 12, 13, 14, 15, 16, 17, 18, 19 or 20 different solubilized peptide antigens with the lipid vesicle particles.

29. The method of any one of claims 26 to 28, wherein, after step (b), each of the different solubilized peptide antigens is at a concentration of at least about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml or 1.0 mg/ml.

30. The method of any one of claims 26 to 29, wherein, after step (b), each of the different solubilized peptide antigens is at a concentration of about 0.5 mg/ml.

31. The method of any one of claims 26 to 30, wherein the different solubilized peptide antigens are not pre-selected based on any characteristic relating to isoelectric point, solubility, stability and/or immunogenicity.

32. The method of any one of claims 26 to 31, wherein the different solubilized peptide antigens have one or more different characteristics relating to isoelectric point, solubility, stability and/or immunogenicity.

33. The method of any one of claims 26 to 32, wherein the different solubilized peptide antigens have a different length, sequence, molecular weight, charge, polarity, hydrophobicity and/or hydrophilicity.

34. The method of any one of claims 20 to 33, wherein step (b) further comprises mixing, in any order, a solubilized T-helper epitope with the lipid vesicle particles and the one or more peptide antigens.

35. The method of claim 33, wherein step (b) comprises mixing 10-15 neoantigens with one solubilized T-helper epitope, wherein the T-helper epitope comprises the amino acid sequence AQYIKANSKFIGITEL (SEQ ID NO: 1).

36. The method of any one of claims 20 to 35, wherein step (b) further comprises mixing, in any order, an adjuvant with the lipid vesicle particles and the one or more peptide antigens.

37. The method of claim 34 or 35, wherein step (b) comprises:
(b1) providing an antigen stock comprising the one or more peptide antigens and the solubilized T-helper epitope; and
(b2) mixing the antigen stock with the lipid vesicle particles to form the mixture.

38. The method of claim 37, wherein, in step (b1), the antigen stock is prepared in 100 mM sodium hydroxide with each solubilized antigen having a concentration of about 2.0 mg/ml.

39. The method of claim 38, wherein the antigen stock is diluted 1:1 with 50 mM sodium acetate having a pH of 6.0±0.5 to provide each solubilized antigen at a concentration of about 1.0 mg/ml.

40. The method of any one of claims 37 to 39, wherein after the mixing in step (b2) and prior to drying, the pH of the mixture is adjusted to 10±1.0.

41. The method of any one of claims 37 to 40, wherein step (b2) further comprises mixing the mixture with an adjuvant.

42. The method of claim 36 or 41, wherein the adjuvant is a polyI:C nucleotide adjuvant.

43. The method of any one of claims 1 to 42 further comprising a step of sterile filtration of the mixture formed in step (b) prior to drying.

44. The method of any one of claims 1 to 43 further comprising, between steps (b) and (c), a step of confirming that the lipid vesicle particles still have a mean particle size of ≤120 nm and a polydispersity index (PDI) of ≤0.1.

45. The method of any one of claims 1 to 44, wherein the drying is performed by lyophilization, spray freeze-drying, or spray drying.

46. The method of claim 45, wherein the drying is performed by lyophilization.

47. The method of claim 46, wherein the lyophilization is performed by loading one or more containers comprising the mixture of step (b) into a bag, sealing the bag to form a sealed unit, and lyophilizing the sealed unit in a freeze-dryer.

48. The method of claim 47, wherein the bag is a sterile, autoclaved bag.

49. The method of claim 47 or 48, wherein the freeze-dryer is a benchtop freeze dryer.

50. The method of any one of claims 47 to 49, wherein the freeze-dryer contains more than one sealed unit during the lyophilization.

51. The method of claim 50, wherein each sealed unit contains a different mixture prepared by steps (a) and (b).

52. The method of any one of claims 1 to 51, further comprising a step of evaluating the stability of the at least one solubilized therapeutic agent before and/or after the drying of step (c).

53. The method of claim 52, wherein the stability of the therapeutic agents is evaluated by HPLC analysis.

54. The method of claim 52 or 53, wherein the therapeutic agents are peptide antigens and at least 80% of the original peptide concentration of each peptide antigen is retained in undegraded form when evaluated before drying.

55. The method of claim 54, wherein at least 75% of the original peptide concentration of each peptide antigen is retained in undegraded form when evaluated immediately after drying.

56. The method of claim 54 or 55, wherein at least 70% of the original peptide concentration of each peptide antigen is retained in undegraded form when evaluated three months after drying.

57. The method of any one of claims 54 to 56, wherein one or more of the peptide antigens shows no degradation for up to 3 months after drying.

58. A method for preparing a pharmaceutical composition comprising solubilizing the dried preparation obtained by the method of any one of claims 1 to 57 in a hydrophobic carrier.

59. The method of claim 58, wherein the hydrophobic carrier is mineral oil or a mannide oleate in mineral oil solution.

60. The method of claim 58 or 59, wherein the hydrophobic carrier is Montanide® ISA 51.

61. A pharmaceutical composition prepared by the method of any one of claims 58 to 60.

62. The pharmaceutical composition of claim 61, wherein the lipids are in the form of one or more lipid-based structures having a single layer lipid assembly in the hydrophobic carrier.

63. The pharmaceutical composition of claim 62, wherein, in the hydrophobic carrier, the lipids are in the form of reverse micelles and/or aggregates of lipids with the hydrophobic part of the lipids oriented outwards toward the hydrophobic carrier and the hydrophilic part of the lipids aggregating as a core.

64. The pharmaceutical composition of claim 62 or 63, wherein the size of the lipid-based structures is between about 2 nm to about 10 nm in diameter.

65. A stable, water-free pharmaceutical composition comprising one or more lipid-based structures having a single layer lipid assembly, at least one therapeutic agent, and a hydrophobic carrier.

66. The pharmaceutical composition of claim 65, wherein the therapeutic agent is a peptide antigen, a DNA or RNA polynucleotide that encodes a polypeptide, a hormone, a cytokine, an allergen, a catalytic DNA (deoxyribozyme), a catalytic RNA (ribozyme), an antisense RNA, an interfering RNA, an antagomir, a small molecule drug, a biologic drug, an antibody, or a fragment or derivative of any one thereof or a mixture thereof.

67. The pharmaceutical composition of claim 65 or 66, wherein the therapeutic agent is one or more peptide antigens.

68. The pharmaceutical composition of claim 67, wherein the one or more peptide antigens are 20-30 amino acids in length.

69. The pharmaceutical composition of claim 67 or 68, wherein the one or more peptide antigens are neoantigens.

70. The pharmaceutical composition of claim 67, wherein the one or more peptide antigens are derived from human papillomavirus (HPV), human immunodeficiency virus (HIV), respiratory syncytial virus (RSV), Bacillus anthracis, Plasmodium, or a survivin polypeptide.

71. The pharmaceutical composition of claim 70, wherein the one or more peptide antigens are FTELTLGEF (SEQ ID NO: 4), LMLGEFLKL (SEQ ID NO: 5), RISTFKNWPK (SEQ ID NO: 6), STFKNWPFL (SEQ ID NO: 7) or LPPAWQPFL (SEQ ID NO: 8); or any combination thereof.

72. The pharmaceutical composition of claim 70, wherein the one or more peptide antigens are NKLCEYNVFHNKTFELPRARVNT (SEQ ID NO: 2) and/or NKLSEHKTFCNKTLEQGQMYQINT (SEQ ID NO: 3).

73. The pharmaceutical composition of any one of claims 67 to 70, which comprises five or more different peptide antigens.

74. The pharmaceutical composition of claim 73, which comprises up to 30 different peptide antigens.

75. The pharmaceutical composition of claim 73, which comprises 10, 11, 12, 13, 14, 15, 16, 17, 18, 19 or 20 different peptide antigens.

76. The pharmaceutical composition of any one of claims 73 to 75, wherein each of the peptide antigens is, independently, at a concentration of between about 0.1 μg/μl and about 5.0 μg/μl.

77. The pharmaceutical composition of any one of claims 73 to 76, wherein each of the peptide antigens is, independently, at a concentration of about 0.25 μg/μl, about 0.5 μg/μl, about 0.75 μg/μl, about 1.0 μg/μl, about 1.25 μg/μl, about 1.5 μg/μl, about 1.75 μg/μl, about 2.0 μg/μl, about 2.25 μg/μl or about 2.5 μg/μl.

78. The pharmaceutical composition of any one of claims 73 to 77, which comprises 10 or more different peptide antigens, each at a concentration of at least about 0.5 μg/μl.

79. The pharmaceutical composition of any one of claims 73 to 78, wherein the peptide antigens are not pre-selected based on any characteristic relating to isoelectric point, solubility, stability and/or immunogenicity.

80. The pharmaceutical composition of any one of claims 73 to 79, wherein the peptide antigens have one or more different characteristics relating to isoelectric point, solubility, stability and/or immunogenicity.

81. The pharmaceutical composition of any one of claims 73 to 80, wherein the peptide antigens have a different length, sequence, molecular weight, charge, polarity, hydrophobicity and/or hydrophilicity.

82. The pharmaceutical composition of any one of claims 65 to 81, further comprising one or both of a T-helper epitope and an adjuvant.

83. The pharmaceutical composition of any one of claims 65 to 82, wherein the hydrophobic carrier is mineral oil or a mannide oleate in mineral oil solution.

84. The pharmaceutical composition of any one of claims 65 to 83, wherein the hydrophobic carrier is Montanide® ISA 51.

85. The pharmaceutical composition of any one of claims 65 to 84, wherein the one or more lipid-based structures having a single layer lipid assembly comprise aggregates of lipids with the hydrophobic part of the lipids oriented outwards toward the hydrophobic carrier and the hydrophilic part of the lipids aggregating as a core.

86. The pharmaceutical composition of any one of claims 65 to 85, wherein the one or more lipid-based structures having a single layer lipid assembly comprise reverse micelles.

87. The pharmaceutical composition of any one of claims 65 to 86, wherein the size of the lipid-based structures is between about 2 nm to about 10 nm in diameter.

88. The pharmaceutical composition of any one of claims 65 to 87, wherein one or more of the at least one therapeutic agent are inside the lipid-based structures.

89. The pharmaceutical composition of any one of claims 65 to 88, wherein one or more of the at least one therapeutic agent are outside the lipid-based structures.

90. The pharmaceutical composition of any one of claims 65 to 89, which is a clear solution.

91. The pharmaceutical composition of any one of claims 65 to 90, which has no visible precipitate.

92. A stable, water-free pharmaceutical composition comprising one or more lipid-based structures having a single layer assembly, five or more different peptide neoantigens, and a hydrophobic carrier, wherein the peptide neoantigens are not pre-selected based on any characteristic relating to isoelectric point, solubility, stability and/or immunogenicity.

93. The pharmaceutical composition of claim 92, wherein the neoantigens have a different length, sequence, molecular weight, charge, polarity, hydrophobicity and/or hydrophilicity.

94. The pharmaceutical composition of claim 92 or 93, wherein the one or more lipid-based structures having a single layer lipid assembly comprise reverse micelles and/or aggregates of lipids with the hydrophobic part of the lipids oriented outwards toward the hydrophobic carrier and the hydrophilic part of the lipids aggregating as a core.

95. The pharmaceutical composition of any one of claim 92 or 94, wherein the size of the lipid-based structures is between about 2 nm to about 10 nm in diameter.

96. A method of inducing an antibody and/or CTL immune response in a subject comprising administering to the subject the pharmaceutical composition of any one of claims 61 to 95.

97. The method of claim 96, which is for treating cancer or an infectious disease.

98. Use of the pharmaceutical composition of any one of claims 61 to 95 for inducing an antibody and/or CTL immune response in a subject.

99. The use of claim 98, which is for the treatment of cancer or an infectious disease.

100. A kit for preparing a pharmaceutical composition for inducing an antibody and/or CTL immune response, the kit comprising:
a container comprising a dried preparation prepared by the method of any one of claims 1 to 57; and
a container comprising a hydrophobic carrier.

101. The kit of claim 100, wherein the dried preparation comprises ten or more different peptide antigens.

102. The kit of claim 100 or 101, wherein the hydrophobic carrier is mineral oil or a mannide oleate in mineral oil solution.
